(19) United States
(12) Patent Application Publication
(10) Pub. No.: US 2026/0109741 A1
(43) Pub. Date: Apr. 23, 2026

(54) STING-TARGETING GENE THERAPY

(71) Applicant: University of Florida Research Foundation, Incorporated, Gainesville, FL (US)

(72) Inventor: Lei Jin, Gainesville, FL (US)

(73) Assignee: University of Florida Research Foundation, Incorporated, Gainesville, FL (US)

(21) Appl. No.: 19/365,352

(22) Filed: Oct. 22, 2025

Related U.S. Application Data
(60) Provisional application No. 63/710,288, filed on Oct. 22, 2024.

(51) Int. Cl. C07K 14/47; A61P 37/00; C12N 15/86

(52) U.S. Cl. CPC C07K 14/47; A61P 37/00; C12N 15/86

(57) ABSTRACT

Provided herein are nucleic acids containing dominant negative STING alleles and compositions containing the same. Further provided are methods of using the nucleic acids containing dominant negative STING alleles for treating STING-associated vasculopathy with onset in infancy (SAVI). Also provided are methods of delivering the nucleic acids containing dominant negative STING alleles to treat STING-mediated conditions or diseases in human subjects in need thereof.

### RELATED APPLICATION

This application claims the benefit under 35 U.S.C. 119 (e) of U.S. Provisional Application Ser. No. 63/710,288, filed Oct. 22, 2024, entitled “STING-TARGETING GENE THERAPY,” the entire contents of which are incorporated by reference herein.

### STATEMENT REGARDING FEDERALLY SPONSORED RESEARCH OR DEVELOPMENT

This invention was made with government support under Grant No. HL 152163, awarded by the National Institutes of Health. The government has certain rights in the invention.

### FIELD

The disclosure is related to the field of STING-mediated diseases and disorders.

### REFERENCE TO AN ELECTRONIC SEQUENCE LISTING

The contents of the electronic sequence listing (U119770251US01-SEQ-JXV.xml; Size: 79,813 bytes; and Date of Creation: Oct. 20, 2025) are herein incorporated by reference in their entirety.

### BACKGROUND

STING (also known as Met-Pro-Tyr-Ser (MPYS), Stimulator of interferon response cGMP interactor 1 (STING1), Endoplasmic reticulum interferon stimulator (ERIS), Mediator of IRF3 activation (MITA) or Transmembrane protein 173 (TMEM173)) is a four transmembrane endoplasmic protein present in the endoplasmic reticulum (ER) which acts as a sensor of the presence of double-stranded DNA (dsDNA) in the cytosol. STING activates TANK-binding kinase 1 (TBK1) leading to phosphorylation and activation of interferon regulatory transcription factor 3 (IRF3) and upregulation of type I interferon (IFN) expression. STING also recruits IKB kinase (IKK) and activates the NF-KB pathway which promotes the production of proinflammatory molecules (including IL-6 and TNFa) and cooperates with TBK1-IRF3 in the production of type I IFN (see, Li et al. Journal of Hematology & Oncology 2019, 12:35). Type I IFN stimulates immune-effector cells including dendritic cells (DC) and natural killer (NK) cells and promotes the production of chemokines like CXCL9 and CXCL10 which attract T lymphocytes, e.g., to tumors (see, Mowat, C. et al J. Exp. Med. 2021 Vol. 218 No. 9 e20210108). STING agonists like cyclic dinucleotides (CDN) have been shown to exert potent antitumor effects in different tumor models (see, Sivick K E et al Cell Reports 2018:25, 3074-3085). STING activators such as 5,6-Dimethylxanthenone-4-acetic acid (DMXAA) have been used in clinical cancer trial but failed due to a lack of efficacy. It was subsequently shown that DMXAA potently activates murine STING but is unable to activate human STING due to amino acid differences of the cyclic-dinucleotide (CDN)-binding site of STING.

### SUMMARY

The present disclosure is based at least in part on the development of nucleic acids, vectors and viral particles, e.g., AAV particles and methods of preparing and using the same to target STING-mediated human diseases or disorders. In some aspects, the nucleic acids, vectors, and viral particles are used in methods that administer the nucleic acids, vectors, and viral particles to a subject in need thereof to suppress an active STING allele. In some aspects, nucleic acids, vectors, and viral particles comprise a dominant STING allele that suppresses an active STING allele, e.g., a gain-of-function mutant STING allele. In some aspects, the nucleic acids, vectors, and viral particles, e.g., AAV particles, and methods provided herein, can also be used to treat a disease or condition related to an activated STING pathway.

Provided in some aspects is a nucleic acid molecule comprising a dominant negative STING allele and an inverted terminal repeat (ITR).

In some aspects, the ITR is an AAV ITR.

In some aspects, the dominant negative STING allele is flanked by two AAV ITRs.

In some aspects, the nucleic acid molecule is a DNA.

In some aspects, the nucleic acid molecule is single stranded.

In some aspects, the nucleic acid molecule is double stranded.

In some aspects, the dominant negative STING allele is selected from a R71H-G230A-R293Q (HAQ) allele, a G230A-R293Q (AQ) allele, a R293Q (Q) allele, and a C148R allele.

In some aspects, the nucleic acid molecule further comprises a regulatory sequence.

In some aspects, the regulatory sequence is a promoter, a termination signal, or a combination thereof.

In some aspects, the dominant negative STING allele is operably linked to a promoter.

Further provided is an AAV particle comprising a nucleic acid molecule described herein.

Also provided is a composition comprising a nucleic acid molecule described herein or an AAV particle described herein.

Further provided is a method of suppressing a gain-of-function mutant STING allele in a cell, the method comprising contacting a cell having a gain-of-function mutant STING allele with an effective amount of a nucleic acid molecule described herein, an AAV particle described herein, or a composition described herein.

In some aspects, the cell is selected from an immune cell and a non-immune cell.

Also provided is a method of suppressing a gain-of-function mutant STING allele in a subject, the method comprising administering to a subject having a gain-of-function mutant STING allele a therapeutically effective amount of a nucleic acid molecule described herein, an AAV particle described herein, or a composition described herein.

In some aspects, the subject is a subject determined to have a gain-of-function mutant STING allele.

Further provided is a method of treating STING-associated vasculopathy with onset in infancy (SAVI), the method comprising administering to a subject in need thereof a therapeutically effective amount of a nucleic acid molecule described herein, an AAV particle described herein, or a composition described herein.

Also provided is a method of suppressing a STING-mediated inflammatory response in a subject, the method comprising administering to the subject a therapeutically effective amount of a nucleic acid molecule described herein, an AAV particle described herein, or a composition described herein.

In some aspects, the subject is a subject determined to have a STING-mediated inflammatory response.

Also provided is a method of treating a STING-mediated inflammatory disease or disorder in a subject, the method comprising administering to the subject a therapeutically effective amount of a nucleic acid molecule described herein, an AAV particle described herein, or a composition described herein. In some embodiments, the STING-mediated inflammatory disease or disorder is a neurodegenerative disease (e.g., Alzheimer's disease, Parkinson's disease, or amyotrophic lateral sclerosis).

In some aspects, the subject is a subject determined to have a STING-mediated disease or disorder. In some embodiments, the STING-mediated disease or disorder is Alzheimer's disease, Parkinson's Disease, or amyotrophic lateral sclerosis.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific aspects presented herein.

FIGS. 1A-1G show that splenocytes from HAQ, AQ, and Q293 mice are resistant to STING-mediated cell death ex vivo. FIG. 1A shows cell death determined by PI staining in CD4 T, CD8 T, and CD19 B cells of C57BL/6N splenocytes treated with diABZI, RpRpssCDA, 2′3′-cGAMP, or DMXAA. FIG. 1B shows cell death of total mouse splenocytes pre-treated with indicated small molecules. FIG. 1C shows flow cytometry blots of PI stained HAQ, AQ, IFNAR1−/− or C57BL/6N splenocytes treated with diABZI. FIG. 1D shows bar graphs of the flow cytometry data of FIG. 1C. FIG. 1E shows bar graphs of flow cytometry data of PI stained wild-type (WT) and Q293 splenocytes shown in FIG. 1F. FIG. 1F shows flow cytometry blots of PI stained wild-type (WT) and Q293 splenocytes treated with diABZI, RpRpssCDA or 2′3′-cGAMP. FIG. 1G shows bar graphs of percent dead WT/HAQ, WT/AQ, or WT/WT splenocytes treated with DMXAA. Data are representative of three independent experiments. Graphs represent the mean with error bars indication s.e.m. p values are determined by one-way ANOVA Tukey's multiple comparison test (FIGS. 1A, 1E, 1G) or unpaired student T-test (FIGS. 1B, 1D). *p<0.05. n.s: not significant.

FIGS. 2A-2I show that HAQ, AQ, and Q293 human cells are resistant to STING agonists-induced death. FIG. 2A shows cell death determined by PI staining in CD4 T, CD8 T. and CD19 B cells of human lung cells from WT/WT individuals treated with diABZI. FIG. 2B shows bar graphs of flow cytometry blots of FIG. 2A. FIG. 2C shows percent dead CD4 T cells of total lung cells from a WT/HAQ (2 individuals) and a WT/WT (3 individuals) treated with diABZi. FIG. 2D shows flow cytometry blots of dead cells determined by Annexin V staining of STING KO THP-1 cells stably reconstituted with human WT (R232), HAQ, AQ, or Q293 alleles following treatment with diABZI. FIG. 2E shows bar graphs of percent Annexin V positive, dead THP-1 WT, STING-KO THP-1 cells, and STING-KO THP-1 cells stably reconstituted with human WT (R232), HAQ, AQ, or Q293 alleles following treatment with diABZI. FIG. 2F shows a line graph of percent Annexin V positive dead STING-KO THP-1 cells, and STING-KO THP-1 cells stably reconstituted with human WT (R232) or Q293 alleles following treatment with diABZI. FIG. 2G shows western blots of STING activation detected by anti-STING antibody in STING-KO THP-1 cells stably reconstituted with human WT (R232) or Q293 alleles following treatment with different doses of diABZI. FIG. 2H shows bar graphs of STING-KO THP-1 cells stably reconstituted with human WT (R232), HAQ, AQ, or Q293 alleles following treatment with diABZI. Pro-apoptotic interferon stimulated gene-54 (ISG-54) reporter luciferase activity was determined in cell supernatant and normalized to 10 ng/ml IFNβ-stimulated ISG-54 luciferase activity. FIG. 2I shows western blots of STING and IRF3 activation determined by anti-STING antibody and anti-p IRF3 antibody (CST, Ser396, clone 4D4G) in STING-KO THP-1 cells stably reconstituted with human WT (R232), HAQ, or AQ alleles. Densitometry was determined by ImageLab 5; data are representative of three independent experiments. Graphs represent the mean with error bars indication s.e.m. p values determined by one-way ANOVA Tukey's multiple comparison test (FIGS. 2B, 2C, 2F, 2H, 2G) or unpaired student T-test (FIGS. 2E, 2I). *p<0.05, n.s: not significant.

FIGS. 3A-3H show that HAQ and AQ rescue the lymphopenia and suppress myeloid cell expansion in SAVI(N153S) mice. FIG. 3A shows the size and weight of spleens from WT/HAQ, HAQ/SAVI, AQ/SAVI, WT/AQ, or WT/SAVI mice. FIG. 3B shows numbers of spleen B cells in WT/HAQ, HAQ/SAVI, AQ/SAVI, WT/AQ, or WT/SAVI mice. FIG. 3C shows flow cytometry blots and bar graphs of CD4 T cells of total spleen cells of WT/HAQ, HAQ/SAVI, AQ/SAVI, WT/AQ, or WT/SAVI mice. FIG. 3D shows flow cytometry blots and bar graphs of CD8 T cells of total spleen cells of WT/HAQ, HAQ/SAVI, AQ/SAVI, WT/AQ, or WT/SAVI mice. FIG. 3E shows flow cytometry blots of Ly6G+ neutrophils and Ly6Chi monocytes of total spleen cells of WT/HAQ, HAQ/SAVI, AQ/SAVI, WT/AQ, or WT/SAVI mice. FIG. 3F shows a bar graph of Ly6G+ cell numbers of WT/HAQ, HAQ/SAVI, AQ/SAVI, WT/AQ, or WT/SAVI mice. FIG. 3G shows a bar graph of cell numbers of Ly6Chi spleen monocytes of WT/HAQ, HAQ/SAVI, AQ/SAVI, WT/AQ, or WT/SAVI mice. FIG. 3H shows a bar graph of cell numbers of F4/80+ spleen macrophages of WT/HAQ, HAQ/SAVI, AQ/SAVI, WT/AQ, or WT/SAVI mice. Data are representative of three independent experiments. n=3-5 mice/group. Graphs represent the mean with error bars indication s.e.m. P values are determined by one-way ANOVA Tukey's multiple comparison test. *p<0.05, n.s: not significant.

FIGS. 4A-4K show that the HAQ and AQ alleles prevent SAVI(N153S) disease in mice. FIG. 4A shows the size of WT/HAQ, HAQ/SAVI, WT/AQ, WT/SAVI, or AQ/SAVI mice. FIG. 4B shows body weight of WT/WT, WT/SAVI, WT/HAQ, or HAQ/SAVI mice. FIG. 4C shows probability of survival of WT/HAQ, WT/SAVI or HAQ/SAVI mice. FIG. 4D shows airway resistance of WT/WT, WT/SAVI, WT/HAQ, or HAQ/SAVI mice. FIG. 4E shows pulmonary artery pressure of WT/WT, WT/SAVI, WT/HAQ, or HAQ/SAVI mice. FIG. 4F shows probability of survival of WT/AQ, WT/SAVI or AQ/SAVI mice. FIG. 4G shows body weight of WT/WT, WT/SAVI, WT/AQ, or AQ/SAVI mice. FIG. 4H shows airway resistance of WT/WT, WT/SAVI, WT/AQ, or AQ/SAVI mice. FIG. 4I shows pulmonary artery pressure of WT/WT, WT/SAVI, WT/AQ, or AQ/SAVI mice. FIG. 4J shows representative hematoxylin and eosin (H&E) staining of lung sections from indicated mice. FIG. 4K shows representative hematoxylin and eosin (H&E) staining of liver sections from indicated mice. n=35 mice/group. Data are representative of 3 independent experiments. Graphs represent the mean with error bars indication s.e.m. p values are determined by one-way ANOVA Tukey's multiple comparison test. *p<0.05. **p<0.01. n.s.: not significant. WBC: white blood cells; 787 H: hepatocytes; K: Kupffer cells; PV: portal vein; CV: central vein.

FIGS. 5A-5F show that AQ/SAVI cells had similar TBK1-IRF3, NFκB activation and STING degradation as the WT/SAVI cells. FIG. 5A shows western blots and bar graphs of BMDM cells from WT/WT, WT/SAVI, AQ/SAVI or HAQ/SAVI mice treated with diABZi; the western blot was probed for phospho-Thr172-TBK1 antibody, phosphor-Ser 396-IRF3, and IκBα antibody FIG. 5B shows western blots and a bar graph of BMDM cells from WT/WT, WT/SAVI, AQ/SAVI or HAQ/SAVI mice treated with diABZi, the western blot was probed for STING antibody. FIG. 5C shows a bar graph of IFNβ ELISA results in BMDM from WT/WT, WT/SAVI, AQ/SAVI or HAQ/SAVI mice treated with diABZi. FIG. 5D shows a bar graph of TNF ELISA results in BMDM from WT/WT, WT/SAVI, AQ/SAVI or HAQ/SAVI mice treated with diABZi. FIG. 5E shows western blots and a bar graph of BMDM cells from WT/WT, WT/SAVI, AQ/SAVI or HAQ/SAVI mice treated with diABZi; the western blot was probed for IκBα. FIG. 5F shows a western blot of BMDM cells from WT/WT, WT/SAVI, HAQ/SAVI or AQ/SAVI mice treated with diABZi; the western blot was probed for STING antibody (Proteintech, 19851-1-AP) and shows STING monomer and dimer. Densitometry was determined by ImageLab. Data are representative of three independent experiments. Graphs represent the mean with error bars indication s.e.m. p values are determined by unpaired student T-test (FIGS. 5A-5E) or one-way ANOVA Tukey's multiple comparison test (FIGS. 5D, 5E). *p<0.05. **p<0.01, ***p<0.001. n.s.: not significant; n.d: not detected.

FIGS. 6A-6B show that HAQ/SAVI and AQ/SAVI cells had 10-fold and 20-fold increased spleen T-regs compared to WT/SAVI mice. FIG. 6A shows flow cytometry analysis and a bar graph of IFNγ producing spleen CD4+ T cells from WT/HAQ, HAQ/SAVI, AQ/SAVI, WT/AQ, or WT/SAVI mice. FIG. 6B shows flow cytometry analysis and a bar graph of CD4+ FoxP3+ spleen T-regs from WT/HAQ, HAQ/SAVI, AQ/SAVI, WT/AQ, or WT/SAVI mice. n=3-5 mice/group. Data are representative of 3 independent experiments. Graphs represent the mean with error bars indication s.e.m. p values are determined by one-way ANOVA Tukey's multiple comparison test. *p<0.05, **p<0.01, ***p<0.001. n.s.: not significant.

FIGS. 7A-7D show that Bx-795 inhibits diABZI-induced mouse splenocyte death. FIG. 7A shows splenocytes from C57BL/6N mice treated with 100 ng/ml diABZI in culture for the indicated time. Dead cells were determined by PI and Annexin V staining. FIG. 7B shows splenocytes from C57BL/6N mice treated with indicated dose of diABZi in culture for 24 hrs. Dead cells were determined by PI staining. FIG. 7C shows splenocytes from C57BL/6N mice pre-treated with indicated small molecules, H151 (10 μg/ml), C176 (1 μM), MCC950 (1.25 μM), 2-BP (20 μM), GSK872 (312.5 nM), Liproxstatin-1 (1 μM), necrostatin-1 (10 μM), VX-765 (1 μM), Z-DEVD-FMK (25 μM), or Bx-795 (0.5 μM) for 2 hrs. diABZi (100 ng/ml) was added in culture for another 24 hrs. Dead cells were determined by PI staining. (FIG. 7D) shows IFNβ determined in the cell supernatant from FIG. 7C by ELISA. Data are representative of three independent experiments. Graphs represent the mean with error bars indication s.e.m. n=3-5 mice/group. p values are determined by one-way ANOVA Tukey's multiple comparison test (FIGS. 7A, 7D) or unpaired student T-test (FIG. 7C). *p<0.05. n.s: not significant. n.d: not detected.

FIGS. 8A-8D show STING activation in primary human cells and THP-1 cells reconstituted with WT human STING. FIG. 8A shows total human lung cells from WT/WT individuals treated with indicated dose of diABZi for 24 hrs. Cell death in CD4, CD8 T cells and CD19 B cells was determined by PI staining. FIG. 8B shows sequencing results of the human TMEM173 gene in WT/WT or WT/HAQ individuals. FIG. 8C-8D show STING-KO THP-1 cells stably reconstituted with human WT (R232) treated with indicated dose of diABZI for indicated time in culture. ISG-54 reporter luciferase activity was determined in cell supernatant and normalized to 10 ng/ml IFNβ-stimulated ISG-54 luciferase activity. Data are representative of at least two independent experiments. Graphs represent the mean with error bars indication s.e.m. p values determined by one-way ANOVA Tukey's multiple comparison test. *p<0.05, n.s: not significant.

FIGS. 9A-9D show HAQ and AQ restore bone marrow monocytes in SAVI (153S) mice. FIG. 9A shows flow analysis of Ly6G⁺CD11B⁺ neutrophils and Ly6G⁻Ly6C⁺CD11B⁺ bone marrow monocytes from HAQ/SAVI, AQ/SAVI, WT/SAVI and their littermates. FIG. 9B shows percent bone marrow neutrophils of bone marrow cells. FIG. 9C shows percent Ly6C^{hi} monocytes of Ly6G bone marrow cells. FIG. 9D shows percent Ly6C^{low} monocytes of Ly6G bone marrow cells. n=3-5 mice/group. Data are representative of three independent experiments. Graphs represent the mean with error bars indication s.e.m. P values are determined by one-way ANOVA Tukey's multiple comparison test. *p<0.05, n.s: not significant

FIGS. 10A-10C show that HAQ and AQ suppress lung myeloid cell infiltration in SAVI(N153S) mice. FIG. 10A shows flow analysis of lung Ly6G+CD11B⁺ neutrophils and Ly6G⁻Ly6C⁺CD11B+ monocytes from HAQ/SAVI, AQ/SAVI, WT/SAVI and their littermates WT/HAQ, WT/AQ mice. FIG. 10B shows cell numbers of lung neutrophils. FIG. 10C shows cell numbers of lung Ly6C^{hi} monocytes. n=3-5 mice/group. Data are representative of three independent experiments. Graphs represent the mean with error bars indication s.e.m. P values are determined by one-way ANOVA Tukey's multiple comparison test. *p<0.05.

FIGS. 11A-11I show that the Q293-STING allele cured SAVI in mice. FIGS. 11A-11B are photographs (FIG. 11A) and a bar plot (FIG. 11B) showing the size and weight of spleens isolated from the 2-month-old male mice. Indicated strains (12 mice/group), were monitored for survival for 10 months. FIG. 11C shows the results in a Kaplan-Meier plot. FIG. 11D shows a plot of the body weight of indicated strains of 10-month-old male mice. FIGS. 11E-11G show spleen CD4 T cell levels, CD8 T cell levels, and neutrophil levels as determined in the 10-month-old male mice by Flow. FIGS. 11H-11I show Spleen T regulatory cells (Treg) were determined in the indicated 10-month-old male mice by Flow. Data are representative of three independent experiments. Graphs represent the mean with error bars indicating s.e.m. P-values are determined by one-way ANOVA, Tukey's multiple comparison test (FIG. 11B), or unpaired Student-T test (FIGS. 11D, 11G, 11I). n.s indicates not significant.

FIGS. 12A-12B show the cross-chain N154(SAVI)-C148 interaction is conserved between chSTING-apo and hSTING-apo states. The full-length cryo-EM structure of chSTING-apo (PDB ID: 6NT6) (FIG. 12A), and hSTING dimer (PDB ID: 6NT5) (FIG. 12B) are illustrated by charged amino acids. Th N159-T153/N154-C148 cross-chain interactions are indicated by arrows. The conserved cross-chain L152-L152/V147-V147 interaction is shown. The structure information (6NT6, 6NT5) are from PMID: 30842659, 37535724.

FIGS. 13A-13B show cGAMP activation breaks the N159-T153 cross-chain interaction in chSTING dimer. The full-length cryo-EM structure of chSTING-apo (PDB ID: 6NT6) (FIG. 13A), and cGAMP bound chSTING dimer (PDB ID: 6NT7) (FIG. 13B) are given. The N159-T153 interaction in 6NT6 is highlighted by arrows; this interaction is missing in 6NT7. The structure information (6NT6, 6NT7) is from PMID: 30842659, 37535724.

FIGS. 14A-14C show that AQ/SAVI partially restored the N154-C148 cross-chain interaction in SAVI STING dimer. FIG. 14A shows the full-length cryo-EM structure hSTING dimer (PDB ID: 6NT5). The N154-C148 cross-chain interactions are indicated by arrows. FIG. 14B shows a structural model of the WT/SAVI[N154S] dimer. The conserved cross-chain N154-C148 interactions are completely missing. FIG. 14C shows a structural model of the AQ/SAVI[N154S] dimer. The conserved cross-chain N154-C148 interactions are partially restored (indicated). The structure information (6NT5) is from PMID: 30842659, 37535724.

FIGS. 15A-15C show the design of a new dominant C148R STING mutant to suppress SAVI-STING autoactivation. FIG. 15A shows a structural model of C148R/SAVI[N154S] dimer based on the hSTING 6NT5. Cross-chain N154-C148, S154-R148, R148-C148, and R148-V147 in the C148R/N154S dimer are indicated. FIG. 15B shows the results of an experiment where STING-KO human monocytic cell line THP-1 (Invivogen, cat no. thpd-kostg) stably expressing human WT, WT-N154S, C148R-N154S, or C148R/C148R were treated with diABZI (200 ng/ml) in culture for 24 hours. Dead cells were determined by Annexin V staining. FIG. 15C shows a Western blot of STING expression for cells in FIG. 15B. Data are representative of three independent experiments. Error bars represent mean±SEM. p values were calculated by unpaired Student's T-test *p<0.05. The structure information (6NT5) is from PMID: 30842659.

FIGS. 16A-16B show that rAAV9-AQ restored STING expression in liver of STING-KO mice but not in brain, lung, heart or spleens. rAAV9-STING (AQ) viral particles (5×10¹¹ genomic copies in 50 ul PBS), which expresses the STING gene with the A230 and Q293 mutations, were injected (intravenously) into a STING-deficient mouse (STING-KO). Ten weeks post-injection, STING protein expression was determined in harvested organs by Western blot. FIG. 16A shows results for liver and spleen tissue. FIG. 16B shows results for brain, lung, and heart tissue. Data are representative of three independent experiments.

### DETAILED DESCRIPTION

The present disclosure is based at least in part on the development of therapies to target STING-mediated human diseases or disorders. In some aspects, provided are compounds, compositions, and methods that suppress an active STING allele. In some aspects, the compounds, compositions, and methods comprise a dominant STING allele. In some aspects, the compounds, compositions, and methods comprise a dominant STING allele that suppresses an active STING allele. In some aspects, the compounds, compositions, and methods comprise a dominant STING allele that suppresses a gain-of-function mutant STING allele. In some aspects, the compounds, compositions, and methods comprise a dominant STING allele that suppresses a gain-of-function mutant STING allele of STING-associated vasculopathy with onset in infancy (SAVI).

Further provided herein are nucleic acids comprising a STING allele, e.g., a dominant STING allele. In some aspects, the nucleic acids comprise an AAV genome (e.g., a recombinant AAV genome), an AAV particle comprising the AAV genomes described herein, compositions comprising AAV particles (e.g., infectious AAV particles), and methods of using the AAV particles and/or compositions for treating a disease or condition in a subject, which disease or condition is related to an activated STING pathway are provided. It has been reported that STING promotes inflammation in a variety of inflammatory diseases including nonalcoholic fatty liver disease, nonalcoholic steatohepatitis, kidney injury, neurodegenerative diseases, cardiovascular diseases, obesity, diabetes, and aging. STING-associated vasculopathy with onset in infancy (SAVI), an autosomal dominant, fatal inflammatory disease is caused by gain-of-function human STING mutations. There exists a need in countering STING activation in SAVI patients and in other inflammatory diseases or conditions.

### Nucleic Acids

Provided are nucleic acids comprising alleles of a STING (TMEM173) gene. In some aspects, a nucleic acid comprises a wild-type STING allele. In some aspects, a nucleic acid comprises a wild-type human STING allele. In some aspects, a nucleic acid comprises a wild-type human STING allele of SEQ ID NO: 2. In some aspects, provided are nucleic acids comprising a STING allele having at least one nucleotide substitution compared to a nucleotide sequence of SEQ ID NO: 2, which nucleic acids encode a STING protein having a same protein sequence as a STING protein encoded by a nucleic acid of SEQ ID NO: 2. In some aspects, provided are nucleic acids comprising codon-modified STING alleles, e.g., codon-optimized STING alleles. In some aspects, codon-modified STING alleles are codon-modified (e.g., codon-optimized) for increased expression in a target tissue and/or target cell. In some aspects, the codon-modified STING alleles encode a STING protein having a same protein sequence as a STING protein encoded by a nucleic acid of SEQ ID NO: 2. In some aspects, a nucleic acid provided herein encodes a wild-type STING protein comprising an amino acid sequence of SEQ ID NO: 1. In some aspects, a nucleic acid provided herein encodes a wild-type STING protein consisting of an amino acid sequence of SEQ ID NO: 1.

In some aspects, a nucleic acid encodes a STING protein comprising 1, 2, 3, 4, 5 or more amino acid substitutions. In some aspects, a nucleic acid encodes a STING protein having a dominant negative phenotype. In some aspects, a nucleic acid encodes a STING protein having a dominant negative phenotype comprising 1, 2, 3, 4, 5 or more amino acid substitutions relative to SEQ ID NO: 1 (wild-type STING protein). In some aspects, a nucleic encodes a STING protein comprising 1 amino acid substitution relative to wild-type STING protein (e.g., SEQ ID NO: 1). In some aspects, a nucleic encodes a STING protein comprising 2 amino acid substitutions relative to wild-type STING protein (e.g., SEQ ID NO: 1). In some aspects, a nucleic encodes a STING protein comprising 3 amino acid substitutions relative to wild-type STING protein (e.g., SEQ ID NO: 1). In some aspects, a nucleic encodes a STING protein comprising 4 amino acid substitutions relative to wild-type STING protein (e.g., SEQ ID NO: 1). In some aspects, a nucleic encodes a STING protein comprising 5 amino acid substitutions relative to wild-type STING protein (e.g., SEQ ID NO: 1).

In some aspects, a nucleic acid encodes a STING allele comprising a substitution (e.g., at least one substitution). In some aspects, a STING allele comprises a substitution of at least 1 nucleotide compared to the sequence of SEQ ID NO: 2. In some aspects, a STING allele comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 2.

In some aspects, a nucleic acid comprises a STING allele that is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 2.

In some aspects, a STING allele comprises a R293Q (Q) substitution in the sequence of SEQ ID NO: 1 (wild-type STING protein). In some aspects, provided is a nucleic acid comprising a R293Q STING allele. In some aspects, provided are nucleic acids comprising a R293Q STING allele that is codon-modified, e.g., a codon-optimized R293Q STING allele. In some aspects, a nucleic acid comprising a R293Q STING allele comprises a sequence of SEQ ID NO: 8. In some aspects, a nucleic acid comprising a R293Q STING allele consists of the sequence of SEQ ID NO: 8.

In some aspects, a STING allele comprising a R293Q substitution comprises a substitution of at least one additional nucleotide relative to SEQ ID NO: 8. In some aspects, a STING allele comprising a R293Q substitution comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to a R293Q STING allele encoded by SEQ ID NO: 8. In some aspects, a nucleic acid comprises a R293Q STING allele and is about 60% to about 99%, e.g., about 65% to about 70%, about 71% to about 75%, about 76% to about 80%, about 81% to about 90%, about 91% to about 95%, or about 96% to about 99% identical (e.g., about 60%, about 65%, about 70%, about 75%, about 80%, about 85%, about 90%, about 95%, about 96%, about 97%, about 98%, or about 99%) to a sequence of SEQ ID NO: 8.

In some aspects, a nucleic acid provided herein encodes a R293Q STING protein comprising an amino acid sequence of SEQ ID NO: 7. In some aspects, a nucleic acid provided herein encodes a R293Q STING protein consisting of the amino acid sequence of SEQ ID NO: 7.

In some aspects, a STING allele comprises a G230A-R293Q (AQ) substitution in the sequence of SEQ ID NO: 1 (wild-type STING protein). In some aspects, provided is a nucleic acid comprising a G230A-R293Q STING allele. In some aspects, provided are nucleic acids comprising a G230A-R293Q (AQ) STING allele that is codon-modified, e.g., a codon-optimized G230A-R293Q (AQ) STING allele. In some aspects, a nucleic acid comprising a G230A-R293Q STING allele comprises a sequence of SEQ ID NO: 6. In some aspects, a nucleic acid comprising a G230A-R293Q STING allele consists of the sequence of SEQ ID NO: 6.

In some aspects, a STING allele comprising a G230A-R293Q substitution comprises a substitution of at least one additional nucleotide relative to SEQ ID NO: 6. In some aspects, a STING allele comprising a G230A-R293Q substitution comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to a G230A-R293Q STING allele of SEQ ID NO: 6. In some aspects, a nucleic acid comprises a G230A-R293Q STING allele and is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 6.

In some aspects, a nucleic acid provided herein encodes a G230A-R293Q STING protein comprising an amino acid sequence of SEQ ID NO: 5. In some aspects, a nucleic acid provided herein encodes a G230A-R293Q STING protein consisting of the amino acid sequence of SEQ ID NO: 5.

In some aspects, a STING allele comprises a R71H-G230A-R293Q (HAQ) substitution in the sequence of SEQ ID NO: 1 (wild-type STING protein). In some aspects, provided is a nucleic acid comprising a R71H-G230A-R293Q STING allele. In some aspects, provided are nucleic acids comprising a R71H-G230A-R293Q (HAQ) STING allele that is codon-modified, e.g., a codon-optimized R71H-G230A-R293Q (HAQ) STING allele. In some aspects, a nucleic acid comprising a R71H-G230A-R293Q STING allele comprises a sequence of SEQ ID NO: 4. In some aspects, a nucleic acid comprising a R71H-G230A-R293Q STING allele consists of the sequence of SEQ ID NO: 4.

In some aspects, a STING allele comprising a R71H-G230A-R293Q substitution comprises a substitution of at least one additional nucleotide relative to SEQ ID NO: 4. In some aspects, a STING allele comprising a R71H-G230A-R293Q substitution comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to a R71H-G230A-R293Q STING allele of SEQ ID NO: 4. In some aspects, a nucleic acid comprises a R71H-G230A-R293Q STING allele and is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 4.

In some aspects, a nucleic acid provided herein encodes a R71H-G230A-R293Q STING protein comprising an amino acid sequence of SEQ ID NO: 3. In some aspects, a nucleic acid provided herein encodes a R71H-G230A-R293Q STING protein consisting of the amino acid sequence of SEQ ID NO: 3.

In some aspects, a STING allele comprises a C148R substitution in the sequence of SEQ ID NO: 1 (wild-type STING protein). In some aspects, provided is a nucleic acid comprising a C148R STING allele. In some aspects, provided are nucleic acids comprising a C148R STING allele that is codon-modified, e.g., a codon-optimized C148R STING allele. In some aspects, a nucleic acid comprising a C148R STING allele comprises a sequence of SEQ ID NO: 36. In some aspects, a nucleic acid comprising a C148R STING allele consists of the sequence of SEQ ID NO: 36.

In some aspects, a STING allele comprising a C148R substitution comprises a substitution of at least one additional nucleotide relative to SEQ ID NO: 36. In some aspects, a STING allele comprising a C148R substitution comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to a C148R STING allele of SEQ ID NO: 36. In some aspects, a nucleic acid comprises a C148R STING allele and is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 36.

In some aspects, a nucleic acid provided herein encodes a C148R STING protein comprising an amino acid sequence of SEQ ID NO: 35. In some aspects, a nucleic acid provided herein encodes a C148R STING protein consisting of the amino acid sequence of SEQ ID NO: 35.

Exemplary STING sequences (amino acid and nucleic acid sequences) are provided in Table 1.

In some aspects, a nucleic acid further comprises a regulatory element. Regulatory elements include, but are not limited to, promoters, enhancers, silencers, insulators, response elements, initiation sites, termination signals, and ribosome binding sites. In some aspects, regulatory elements can be one or more naturally occurring and/or engineered regulatory elements.

In some aspects, a nucleic acid comprises a promoter operably linked to a STING allele. The term “operably linked,” as used herein refers to a nucleic acid sequence joined to another nucleic acid sequence as part of the same nucleic acid molecule. For example, a promoter is positioned and oriented for transcription of a STING allele described herein.

In some aspects, a promoter is a constitutive promoter, a tissue- or cell-specific promoter, or an inducible promoter.

In some aspects, a promoter is a constitutive promoter. Non-limiting examples of constitutive promoters include a cytomegalovirus (CMV) promoter (optionally with the CMV enhancer), an EF1a promoter, a Herpes Simplex virus (HSV) promoter, thymidine kinase (TK) promoter, Rous Sarcoma Virus (RSV) promoter, Simian Virus 40 (SV40) promoter, Mouse Mammary Tumor Virus (MMTV) promoter, Ad E1A promoter, dihydrofolate reductase promoter, an actin promoter, a phosphoglycerol kinase (PGK) promoter, or a β-actin promoter.

In some aspects, a promoter is a tissue- or cell-specific promoter. In some aspect, a promoter is an immune cell-specific promoter. In some aspects, immune cell specific promoters include, but are not limited to, a CD3 promoter, a CD45 promoter, a CD43 promoter, a CD4 promoter, a CD8 promoter, a CD68 promoter, a B29 promoter, a CD14 promoter, a GPIIb promoter, an IFNβ promoter, an IFNγ promoter/enhancer, and a WASP promoter.

In some aspects, a promoter is a tissue-specific promoter, such as an endothelial cell-specific promoter or a lung-specific promoter. In some aspect, a promoter is an endothelial cell-specific promoter. In some aspect, endothelial cell-specific promoters include, but are not limited to, an endoglin promoter, a Tie2 promoter, a Flt-1 promoter, and an ICAM-2 promoter.

In some aspects, a promoter is a lung-specific promoter. In some aspect, a lung specific promoter is a SP-B promoter.

In some aspects, a promoter is an inducible promoter. In some aspects, an inducible promoter is regulated by an exogenously supplied compound, an environmental factor such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of ordinary skill in the art. Examples of inducible promoters regulated by exogenously supplied compounds, include, a zinc-inducible metallothionine (MT) promoter, a dexamethasone (Dex)-inducible promoter, cytochrome P450 promoter system, tetVP16 promoter, a T7 polymerase promoter system, an ecdysone insect promoter, a tetracycline-repressible system (see, e.g., Gossen et al, Proc. Natl. Acad. Sci. USA, 89:5547-5551, 1992), a tetracycline-inducible system (see, e.g., Gossen et al, Science, 268:1766-1769, 1995 and Harvey et al, Curr. Opin. Chem. Biol., 2:512-518, 1998), a RU486-inducible system (see, e.g., Wang et al, Nat. Biotech., 15:239-243, 1997, and Wang et al, Gene Ther., 4:432-441, 1997) and a rapamycin-inducible system (see, e.g., Magari et al, J. Clin. Invest., 100:2865-2872, 1997). Other types of inducible promoters which may be useful in this context are those which are regulated by a specific physiological state, e.g., temperature, acute phase, a particular differentiation state of the cell, or in replicating cells only.

In some aspects, a nucleic acid comprises a polyadenylation (polyA) sequence. Examples of poly A sequences include, but are not limited to, a human growth hormone (hGH) poly(A), a bovine growth hormone poly(A), a SV40 poly(A), a rabbit beta-globin poly(A), and a synthetic poly(A).

In some aspects, a nucleic acid comprises a regulatory element for replication of the nucleic acid in a cell. In some aspects, the regulatory element is for replication in a mammalian cell. In some aspects, the regulatory element is for replication in a bacterial cell. In some aspects, the regulatory element is for replication in an insect cell.

In some aspects, a nucleic acid comprises a viral genome. In some aspects, a nucleic acid comprises an adeno-associated virus (AAV) genome, e.g., a recombinant AAV (rAAV) genome. In some aspects, a viral genome, e.g., an AAV genome can be of any AAV serotype, such as AAV1, AAV2, AAV3, AAV4, AAV5, AAV6, AAV7, AAV8, AAV9, AAV10, AAV11, AAV12, AAV13, AAVrh10, AAVrh74, AAV218, Anc80L65, MyoAAV or a combination of serotypes. In some aspects, a nucleic acid comprises native AAV nucleotide sequences. In some aspects, one or more native AAV nucleotide sequences may be removed from a nucleic acid, e.g., a recombinant AAV genome. In some aspects, one or more native AAV nucleotide sequences may be removed from a nucleic acid and replaced with a gene of interest, e.g., a STING allele. The terms “replaced” and “substituted” are used interchangeably herein.

In some aspects, a nucleic acid comprises at least one inverted terminal repeat (ITR). In some aspects, a nucleic acid comprises two ITRs. In some aspects, a nucleic acid comprises an adeno-associated virus (AAV) ITR. In some aspects, a nucleic acid comprises a STING allele flanked by two AAV ITRs. In some aspects, a nucleic acid comprises an AAV ITR of any AAV serotype (e.g., AAV1, AAV2, AAV3, AAV4, AAV5, AAV6, AAV7, AAV8, AAV9, AAV10, AAV11, AAV12, AAV13, AAVrh10, AAVrh74, AAV2i8, Anc80L65, or MyoAAV). In some aspects, both AAV ITRs are of a same AAV serotype. In some aspects, both AAV ITRs are of a different AAV serotype.

In some aspects, a nucleic acid, e.g., an AAV genome is single-stranded and comprises a 5′ITR and a 3′ ITR. As disclosed herein, the 5′ ITR refers to the ITR at the 5′ terminus of the nucleic acid, e.g., a recombinant AAV genome, and the 3′ ITR refers to the ITR at the 3′ terminus of the nucleic acid, e.g., a recombinant AAV genome. Each ITR in its native or wild-type form is or is about 145 nucleotides in length (e.g., about 140 nucleotides, about 145 nucleotides, about 150 nucleotides, about 155 nucleotides, about 160 nucleotides, or about 165 nucleotides). ITRs are described, for example, in Grimm et al. J. Virol. 80 (1): 426-439 (2006).

### Example of Wild-Type AAV2 5′ ITR:

(SEQ ID NO: 9)
TTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGC
GAGCGAGCGCGCAGAGAGGGAGTGGCCAACTCCATCACTAGGGGTTCC
T

In some aspects, a nucleic acid is a DNA. In some aspects, a nucleic acid is an RNA. In some aspects, a nucleic acid is single-stranded. In some aspects, a nucleic acid is double-stranded. In some aspects, a nucleic acid is a single-stranded DNA. In some aspects, a nucleic acid is a double-stranded DNA, e.g., a self-complementary DNA.

In some aspects, a nucleic acid is a single-stranded DNA and comprises at least one AAV ITR. In some aspects, a nucleic acid is a single-stranded DNA and comprises two AAV ITRs. In some aspects, a nucleic acid is a double-stranded DNA, e.g., a self-complementary DNA and comprises a single AAV ITR.

In some aspects, a nucleic acid is a single-stranded DNA and comprises a STING allele and at least one AAV ITR. In some aspects, a nucleic acid comprises a wild-type human STING allele of SEQ ID NO: 2 and at least one AAV ITR. In some aspects, a nucleic acid comprising a STING allele of SEQ ID NO: 2 comprises a substitution of at least 1 nucleotide compared to the sequence of SEQ ID NO: 2 and at least one AAV ITR. In some aspects, a nucleic acid comprising a STING allele of SEQ ID NO: 2 comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 2 and at least one AAV ITR. In some aspects, a nucleic acid comprising a STING allele of SEQ ID NOs: 2, 4, 6, or 8 and at least one AAV ITR. In some aspects, a nucleic acid comprising a STING allele of any one of SEQ ID NOs: 2, 4, 6, or 8 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to any one of SEQ ID NOs: 2, 4, 6, or 8 and at least one AAV ITR.

In some aspects, a nucleic acid comprising a STING allele is about 60% to about 99%. e.g., about 65% to about 70%, about 71% to about 75%, about 76% to about 80%, about 81% to about 90%, about 91% to about 95%, or about 96% to about 99% identical to SEQ ID NO: 2 and comprises at least one AAV ITR. In some aspects, a nucleic acid comprising a STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to any one of SEQ ID NOs: 2, 4, 6, or 8 and comprises at least one AAV ITR.

In some aspects, a nucleic acid is a double-stranded DNA, e.g., a self-complementary DNA and comprises a STING allele and at least one AAV ITR. In some aspects, a self-complementary DNA comprises a wild-type human STING allele of SEQ ID NO: 2 and at least one AAV ITR. In some aspects, a self-complementary DNA comprising a STING allele of SEQ ID NO: 2 comprises a substitution of at least 1 nucleotide compared to the sequence of SEQ ID NO: 2 and at least one AAV ITR. In some aspects, a self-complementary DNA comprising a STING allele of SEQ ID NO: 2 comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 2 and at least one AAV ITR.

In some aspects, a self-complementary DNA comprising a STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 2 and comprises at least one AAV ITR.

In some aspects, a nucleic acid is a single-stranded DNA and comprises a STING allele and at least one AAV ITR. In some aspects, a nucleic acid comprises a R293Q STING allele of SEQ ID NO: 8 and at least one AAV ITR. In some aspects, a nucleic acid comprising a R293Q STING allele of SEQ ID NO: 8 further comprises a substitution of at least 1 nucleotide compared to the sequence of SEQ ID NO: 8 and at least one AAV ITR. In some aspects, a nucleic acid comprising a R293Q STING allele of SEQ ID NO: 8 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 8 and at least one AAV ITR.

In some aspects, a nucleic acid comprising a R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 8 and comprises at least one AAV ITR.

In some aspects, a nucleic acid is a double-stranded DNA, e.g., a self-complementary DNA and comprises a R293Q STING allele and at least one AAV ITR. In some aspects, a self-complementary DNA comprises a R293Q STING allele of SEQ ID NO: 8 and at least one AAV ITR. In some aspects, a self-complementary DNA comprising a R293Q STING allele of SEQ ID NO: 8 further comprises a substitution of at least 1 nucleotide compared to the sequence of SEQ ID NO: 8 and at least one AAV ITR. In some aspects, a self-complementary DNA comprising a R293Q STING allele of SEQ ID NO: 8 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 8 and at least one AAV ITR.

In some aspects, a self-complementary DNA comprising a R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 8 and comprises at least one AAV ITR.

In some aspects, a nucleic acid is a single-stranded DNA and comprises a G230A-R293Q STING allele and at least one AAV ITR. In some aspects, a nucleic acid comprises a G230A-R293Q STING allele of SEQ ID NO: 6 and at least one AAV ITR. In some aspects, a nucleic acid comprising a G230A-R293Q STING allele of SEQ ID NO: 6 further comprises a substitution of at least 1 nucleotide compared to the sequence of SEQ ID NO: 6 and at least one AAV ITR. In some aspects, a nucleic acid comprising a G230A-R293Q STING allele of SEQ ID NO: 6 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 6 and at least one AAV ITR.

In some aspects, a nucleic acid comprising a G230A-R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 6 and comprises at least one AAV ITR.

In some aspects, a nucleic acid is a double-stranded DNA, e.g., a self-complementary DNA and comprises a G230A-R293Q STING allele and at least one AAV ITR. In some aspects, a self-complementary DNA comprises a G230A-R293Q STING allele of SEQ ID NO: 6 and at least one AAV ITR. In some aspects, a self-complementary DNA comprising a G230A-R293Q STING allele of SEQ ID NO: 6 further comprises a substitution of at least 1 nucleotide compared to the sequence of SEQ ID NO: 6 and at least one AAV ITR. In some aspects, a self-complementary DNA comprising a G230A-R293Q STING allele of SEQ ID NO: 6 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 6 and at least one AAV ITR.

In some aspects, a self-complementary DNA comprising a G230A-R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 6 and comprises at least one AAV ITR.

In some aspects, a nucleic acid is a single-stranded DNA and comprises a R71H-G230A-R293Q STING allele and at least one AAV ITR. In some aspects, a nucleic acid comprises a R71H-G230A-R293Q STING allele of SEQ ID NO: 4 and at least one AAV ITR. In some aspects, a nucleic acid comprising a R71H-G230A-R293Q STING allele of SEQ ID NO: 4 further comprises a substitution of at least 1 nucleotide compared to the sequence of SEQ ID NO: 4 and at least one AAV ITR. In some aspects, a nucleic acid comprising a R71H-G230A-R293Q STING allele of SEQ ID NO: 4 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 4 and at least one AAV ITR.

In some aspects, a nucleic acid comprising a R71H-G230A-R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 4 and comprises at least one AAV ITR.

In some aspects, a nucleic acid is a double-stranded DNA, e.g., a self-complementary DNA and comprises a R71H-G230A-R293Q STING allele and at least one AAV ITR. In some aspects, a self-complementary DNA comprises a R71H-G230A-R293Q STING allele of SEQ ID NO: 4 and at least one AAV ITR. In some aspects, a self-complementary DNA comprising a R71H-G230A-R293Q STING allele of SEQ ID NO: 4 further comprises a substitution of at least 1 nucleotide compared to the sequence of SEQ ID NO: 4 and at least one AAV ITR. In some aspects, a self-complementary DNA comprising a R71H-G230A-R293Q STING allele of SEQ ID NO: 4 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 4 and at least one AAV ITR.

In some aspects, a self-complementary DNA comprising a R71H-G230A-R293Q STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 4 and comprises at least one AAV ITR.

In some aspects, a nucleic acid is a single-stranded DNA and comprises a C148R STING allele and at least one AAV ITR. In some aspects, a nucleic acid comprises a C148R STING allele of SEQ ID NO: 36 and at least one AAV ITR. In some aspects, a nucleic acid comprising a C148R STING allele of SEQ ID NO: 36 further comprises a substitution of at least 1 nucleotide compared to the sequence of SEQ ID NO: 36 and at least one AAV ITR. In some aspects, a nucleic acid comprising a C148R STING allele of SEQ ID NO: 36 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 36 and at least one AAV ITR.

In some aspects, a nucleic acid comprising a C148R STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 36 and comprises at least one AAV ITR.

In some aspects, a nucleic acid is a double-stranded DNA, e.g., a self-complementary DNA and comprises a C148R STING allele and at least one AAV ITR. In some aspects, a self-complementary DNA comprises a C148R STING allele of SEQ ID NO: 36 and at least one AAV ITR. In some aspects, a self-complementary DNA comprising a C148R STING allele of SEQ ID NO: 36 further comprises a substitution of at least 1 nucleotide compared to the sequence of SEQ ID NO: 36 and at least one AAV ITR. In some aspects, a self-complementary DNA comprising a C148R STING allele of SEQ ID NO: 36 further comprises a substitution of about 1-15 nucleotides (e.g., 1, 2, 3, 4, 5, 6, 7, 8, 9, 10, 11, 12, 13, 14, or 15 nucleotides), or about 1-5, about 6-10, or about 11-15 nucleotides compared to the sequence of SEQ ID NO: 36 and at least one AAV ITR.

In some aspects, a self-complementary DNA comprising a C148R STING allele is about 60% to about 99%, e.g., about 65% to about 70%, about 71% to about 75%, about 76% to about 80%, about 81% to about 90%, about 91% to about 95%, or about 96% to about 99% (e.g., about 60%, about 65%, about 70%, about 75%, about 80%, about 85%, about 90%, about 95%, about 96%, about 97%, about 98%, or about 99%) identical to SEQ ID NO: 36 and comprises at least one AAV ITR.

In some aspects, a nucleic acid is a double-stranded DNA, e.g., a self-complementary DNA and comprises a C148R STING allele and at least one AAV ITR.

In some aspects, a nucleic acid comprises a nucleotide sequence for genomic integration. In some aspects, a nucleic acid comprising a nucleotide sequence for genomic integration further comprises nucleotide sequences flanking the nucleotide sequence for genomic integration. In some aspects, the nucleotide sequences flanking the nucleotide sequence for genomic integration comprise a 5′ homology arm and a 3′ homology arm that are homologous to a genomic site for genomic integration. In some aspects, the genomic site for genomic integration is in a human genome. In some aspects, the genomic site for genomic integration is in a safe harbor.

In some aspects, the genomic site for genomic integration is an AAVS1.

Exemplary Sequences

| | | SEQ |
|---|---|---|
| | | ID |
| Name | Sequence | NO: |
| Human | MPHSSLHPSIPCPRGHGAQKAALVLLSACLVTLWGLGEPPEHTLR | 1 |
| STING | YLVLHLASLQLGLLLNGVCSLAEELRHIHSRYRGSYWRTVRACLG | |
| Protein | CPLRRGALLLLSIYFYYSLPNAVGPPFTWMLALLGLSQALNILLG | |
| (WT) | LKGLAPAEISAVCEKGNFNVAHGLAWSYYIGYLRLILPELQARIR | |
| | TYNQHYNNLLRGAVSQRLYILLPLDCGVPDNLSMADPNIRFLDKL | |
| | PQQTGDRAGIKDRVYSNSIYELLENGQRAGTCVLEYATPLQTLFA | |
| | MSQYSQAGFSREDRLEQAKLFCRTLEDILADAPESQNNCRLIAYQ | |
| | EPADDSSFSLSQEVLRHLRQEEKEEVTVGSLKTSAVPSTSTMSQE | |
| | PELLISGMEKPLPLRTDFS | |
| Human | atgccccactccagcctgcatccatccatcccgtgtcccaggggt | 2 |
| STING | cacggggcccagaaggcagccttggttctgctgagtgcctgcctg | |
| Nucleic | gtgaccctttgggggctaggagagccaccagagcacactctccgg | |
| Acid | tacctggtgctccacctagcctccctgcagctgggactgctgtta | |
| (WT) | aacggggtctgcagcctggctgaggagctgcgccacatccactcc | |
| | aggtaccggggcagctactggaggactgtgcgggcctgcctgggc | |
| | tgccccctccgccgtggggccctgttgctgctgtccatctatttc | |
| | tactactccctcccaaatgggtcggcccgcccttcacttggatgc | |
| | ttgccctcctgggcctctcgcaggcactgaacatcctcctgggcc | |
| | tcaagggcctggccccagctgagatctctgcagtgtgtgaaaaag | |
| | ggaatttcaacgtggcccatgggctggcatggtcatattacatcg | |
| | gatatctgcggctgatcctgccagagctccaggcccggattcgaa | |
| | cttacaatcagcattacaacaacctgctacggggtgcagtgagcc | |
| | agcggctgtatattctcctcccattggactgtggggtgcctgata | |
| | acctgagtatggctgaccccaacattcgcttcctggataaactgc | |
| | cccagcagaccggtgaccgtgctggcatcaaggatcgggtttaca | |
| | gcaacagcatctatgagcttctggagaacgggcagcgggcgggca | |
| | cctgtgtcctggagtacgccacccccttgcagactttgtttgcca | |
| | tgtcacaatacagtcaagctggctttagccgggaggataggcttg | |
| | agcaggccaaactcttctgccggacacttgaggacatcctggcag | |
| | atgcccctgagtctcagaacaactgccgcctcattgcctaccagg | |
| | aacctgcagatgacagcagcttctcgctgtcccaggaggttctcc | |
| | ggcacctgcggcaggaggaaaaggaagaggttactgtgggcagct | |
| | tgaagacctcagcggtgcccagtacctccacgatgtcccaagagc | |
| | ctgagctcctcatcagtggaatggaaaagcccctccctctccgca | |
| | cggatttctcttga | |
| Human | MPHSSLHPSIPCPRGHGAQKAALVLLSACLVTLWGLGEPPEHTLR | 3 |
| STING | YLVLHLASLQLGLLLNGVCSLAEELHHIHSRYRGSYWRTVRACLG | |
| Protein | CPLRRGALLLLSIYFYYSLPNAVGPPFTWMLALLGLSQALNILLG | |
| (HAQ | LKGLAPAEISAVCEKGNFNVAHGLAWSYYIGYLRLILPELQARIR | |
| allele) | TYNQHYNNLLRGAVSQRLYILLPLDCGVPDNLSMADPNIRFLDKL | |
| | PQQTADRAGIKDRVYSNSIYELLENGQRAGTCVLEYATPLQTLFA | |
| | MSQYSQAGFSREDRLEQAKLFCQTLEDILADAPESQNNCRLIAYQ | |
| | EPADDSSFSLSQEVLRHLRQEEKEEVTVGSLKTSAVPSTSTMSQE | |
| | PELLISGMEKPLPLRTDFS | |
| Human | atgccccactccagcctgcatccatccatcccgtgtcccaggggt | 4 |
| STING | cacggggcccagaaggcagccttggttctgctgagtgcctgcctg | |
| HAQ | gtgaccctttgggggctaggagagccaccagagcacactctccgg | |
| Allele | tacctggtgctccacctagcctccctgcagctgggactgctgtta | |
| | aacggggtctgcagcctggctgaggagctgcaccacatccactcc | |
| | aggtaccggggcagctactggaggactgtggggcctgcctgggct | |
| | gccccctccgccgtggggccctgttgctgctgtccatctatttct | |
| | actactccctcccaaatgcggtcggcccgcccttcacttggatgc | |
| | ttgccctcctgggcctctcgcaggcactgaacatcctcctgggcc | |
| | tcaagggcctggccccagctgagatctctgcagtgtgtgaaaaag | |
| | ggaatttcaacgtggcccatgggctggcatggtcatattacatcg | |
| | gatatctgcggctgatcctgccagagctccaggcccggattcgaa | |
| | cttacaatcagcattacaacaacctgctacggggtgcagtgagcc | |
| | agcggctgtatattctcctcccattggactgtggggtgcctgata | |
| | acctgagtatggctgaccccaacattcgcttcctggataaactgc | |
| | cccagcagaccgctgaccgtgctggcatcaaggatcgggtttaca | |
| | gcaacagcatctatgagcttctggagaacgggcagcggggggcac | |
| | ctgtgtcctggagtacgccacccccttgcagactttgtttgccat | |
| | gtcacaatacagtcaagctggctttagccgggaggataggcttga | |
| | gcaggccaaactcttctgccagacacttgaggacatcctggcaga | |
| | tgcccctgagtctcagaacaactgccgcctcattgcctaccagga | |
| | acctgcagatgacagcagcttctcgctgtcccaggaggttctccg | |
| | gcacctgcggcaggaggaaaaggaagaggttactgtgggcagctt | |
| | gaagacctcagcggtgcccagtacctccacgatgtcccaagagcc | |
| | tgagctcctcatcagtggaatggaaaagcccctccctctccgcac | |
| | ggatttctcttga | |
| Human | MPHSSLHPSIPCPRGHGAQKAALVLLSACLVTLWGLGEPPEHTLR | 5 |
| STING | YLVLHLASLQLGLLLNGVCSLAEELRHIHSRYRGSYWRTVRACLG | |
| Protein | CPLRRGALLLLSIYFYYSLPNAVGPPFTWMLALLGLSQALNILLG | |
| (G230A | LKGLAPAEISAVCEKGNFNVAHGLAWSYYIGYLRLILPELQARIR | |
| -R293Q | TYNQHYNNLLRGAVSQRLYILLPLDCGVPDNLSMADPNIRFLDKL | |
| allele) | PQQTADRAGIKDRVYSNSIYELLENGQRAGTCVLEYATPLQTLFA | |
| | MSQYSQAGFSREDRLEQAKLFCQTLEDILADAPESQNNCRLIAYQ | |
| | EPADDSSFSLSQEVLRHLRQEEKEEVTVGSLKTSAVPSTSTMSQE | |
| | PELLISGMEKPLPLRTDFS | |
| Human | atgccccactccagcctgcatccatccatcccgtgtcccaggggt | 6 |
| STING | cacggggcccagaaggcagccttggttctgctgagtgcctgcctg | |
| G230A- | gtgaccctttgggggctaggagagccaccagagcacactctccgg | |
| R293Q | tacctggtgctccacctagcctccctgcagctgggactgctgtta | |
| Allele | aacggggtctgcagcctggctgaggagctgcgccacatccactcc | |
| | aggtaccggggcagctactggaggactgtgcgggcctgcctgggc | |
| | tgccccctccgccgtggggccctgttgctgctgtccatctatttc | |
| | tactactccctcccaaatgcggtcggcccgcccttcacttggatg | |
| | cttgccctcctgggcctctcgcaggcactgaacatcctcctgggc | |
| | ctcaagggcctggccccagctgagatctctgcagtgtgtgaaaaa | |
| | gggaatttcaacgtggcccatgggctggcatggtcatattacatc | |
| | ggatatctgcggctgatcctgccagagctccaggcccggattcga | |
| | acttacaatcagcattacaacaacctgctacggggtgcagtgagc | |
| | cagcggctgtatattctcctcccattggactgtggggtgcctgat | |
| | aacctgagtatggctgaccccaacattcgcttcctggataaactg | |
| | ccccagcagaccgctgaccgtgctggcatcaaggatcgggtttac | |
| | agcaacagcatctatgagcttctggagaacgggcagcggggggca | |
| | cctgtgtcctggagtacgccacccccttgcagactttgtttgcca | |
| | tgtcacaatacagtcaagctggctttagccgggaggataggcttg | |
| | agcaggccaaactcttctgccagacacttgaggacatcctggcag | |
| | atgcccctgagtctcagaacaactgccgcctcattgcctaccagg | |
| | aacctgcagatgacagcagcttctcgctgtcccaggaggttctcc | |
| | ggcacctgcggcaggaggaaaaggaagaggttactgtgggcagct | |
| | tgaagacctcagcggtgcccagtacctccacgatgtcccaagagc | |
| | ctgagctcctcatcagtggaatggaaaagcccctccctctccgca | |
| | cggatttctcttga | |
| Human | MPHSSLHPSIPCPRGHGAQKAALVLLSACLVTLWGLGEPPEHTLR | 7 |
| STING | YLVLHLASLQLGLLLNGVCSLAEELRHIHSRYRGSYWRTVRACLG | |
| Protein | CPLRRGALLLLSIYFYYSLPNAVGPPFTWMLALLGLSQALNILLG | |
| (R293Q | LKGLAPAEISAVCEKGNFNVAHGLAWSYYIGYLRLILPELQARIR | |
| allele) | TYNQHYNNLLRGAVSQRLYILLPLDCGVPDNLSMADPNIRFLDKL | |
| | PQQTGDRAGIKDRVYSNSIYELLENGQRAGTCVLEYATPLQTLFA | |
| | MSQYSQAGFSREDRLEQAKLFCQTLEDILADAPESQNNCRLIAYQ | |
| | EPADDSSFSLSQEVLRHLRQEEKEEVTVGSLKTSAVPSTSTMSQE | |
| | PELLISGMEKPLPLRTDFS | |
| Human | atgccccactccagcctgcatccatccatcccgtgtcccaggggt | 8 |
| STING | cacggggcccagaaggcagccttggttctgctgagtgcctgcctg | |
| R293Q | gtgaccctttgggggctaggagagccaccagagcacactctccgg | |
| Allele | tacctggtgctccacctagcctccctgcagctgggactgctgtta | |
| | aacggggtctgcagcctggctgaggagctgcgccacatccactcc | |
| | aggtaccggggcagctactggaggactgtgcgggcctgcctgggc | |
| | tgccccctccgccgtggggccctgttgctgctgtccatctatttc | |
| | tactactccctcccaaatgcggtcggcccgcccttcacttggatg | |
| | cttgccctcctgggcctctcgcaggcactgaacatcctcctgggc | |
| | ctcaagggcctggccccagctgagatctctgcagtgtgtgaaaaa | |
| | gggaatttcaacgtggcccatgggctggcatggtcatattacatc | |
| | ggatatctgcggctgatcctgccagagctccaggcccggattcga | |
| | acttacaatcagcattacaacaacctgctacggggtgcagtgagc | |
| | cagcggctgtatattctcctcccattggactgtggggtgcctgat | |
| | aacctgagtatggctgaccccaacattcgcttcctggataaactg | |
| | ccccagcagaccggtgaccgtgctggcatcaaggatcgggtttac | |
| | agcaacagcatctatgagcttctggagaacgggcagcgggcgggc | |
| | acctgtgtcctggagtacgccacccccttgcagactttgtttgcc | |
| | atgtcacaatacagtcaagctggctttagccgggaggataggctt | |
| | gagcaggccaaactcttctgccagacacttgaggacatcctggca | |
| | gatgcccctgagtctcagaacaactgccgcctcattgcctaccag | |
| | gaacctgcagatgacagcagcttctcgctgtcccaggaggttctc | |
| | cggcacctgcggcaggaggaaaaggaagaggttactgtgggcagc | |
| | ttgaagacctcagcggtgcccagtacctccacgatgtcccaagag | |
| | cctgagctcctcatcagtggaatggaaaagcccctccctctccgc | |
| | acggatttctcttga | |
| Human | MPHSSLHPSIPCPRGHGAQKAALVLLSACLVTLWGLGEPPEHTLR | 35 |
| STING | YLVLHLASLQLGLLLNGVCSLAEELRHIHSRYRGSYWRTVRACLG | |
| Protein | CPLRRGALLLLSIYFYYSLPNAVGPPFTWMLALLGLSQALNILLG | |
| (C148R | LKGLAPAEISAVREKGNFNVAHGLAWSYYIGYLRLILPELQARIR | |
| allele) | TYNQHYNNLLRGAVSQRLYILLPLDCGVPDNLSMADPNIRFLDKL | |
| | PQQTGDHAGIKDRVYSNSIYELLENGQRAGTCVLEYATPLQTLFA | |
| | MSQYSQAGFSREDRLEQAKLFCRTLEDILADAPESQNNCRLIAYQ | |
| | EPADDSSFSLSQEVLRHLRQEEKEEVTVGSLKTSAVPSTSTMSQE | |
| | PELLISGMEKPLPLRTDFS | |
| Human | ATGCCCCACTCCAGCCTGCATCCATCCATCCCGTGTCCCAGGGGT | 36 |
| STING | CACGGGGCCCAGAAGGCAGCCTTGGTTCTGCTGAGTGCCTGCCTG | |
| C148R | GTGACCCTTTGGGGGCTAGGAGAGCCACCAGAGCACACTCTCCGG | |
| Allele | TACCTGGTGCTCCACCTAGCCTCCCTGCAGCTGGGACTGCTGTTA | |
| | AACGGGGTCTGCAGCCTGGCTGAGGAGCTGCGCCACATCCACTCC | |
| | AGGTACCGGGGCAGCTACTGGAGGACTGTGCGGGCCTGCCTGGGC | |
| | TGCCCCCTCCGCCGTGGGGCCCTGTTGCTGCTGTCCATCTATTTC | |
| | TACTACTCCCTCCCAAATGCGGTCGGCCCGCCCTTCACTTGGATG | |
| | CTTGCCCTCCTGGGCCTCTCGCAGGCACTGAACATCCTCCTGGGC | |
| | CTCAAGGGCCTGGCCCCAGCTGAGATCTCTGCAGTGCGTGAAAAA | |
| | GGGAATTTCAACGTGGCCCATGGGCTGGCATGGTCATATTACATC | |
| | GGATATCTGCGGCTGATCCTGCCAGAGCTCCAGGCCCGGATTCGA | |
| | ACTTACAATCAGCATTACAACAACCTGCTACGGGGTGCAGTGAGC | |
| | CAGCGGCTGTATATTCTCCTCCCATTGGACTGTGGGGTGCCTGAT | |
| | AACCTGAGTATGGCTGACCCCAACATTCGCTTCCTGGATAAACTG | |
| | CCCCAGCAGACCGGTGACCATGCTGGCATCAAGGATCGGGTTTAC | |
| | AGCAACAGCATCTATGAGCTTCTGGAGAACGGGCAGCGGGCGGGC | |
| | ACCTGTGTCCTGGAGTACGCCACCCCCTTGCAGACTTTGTTTGCC | |
| | ATGTCACAATACAGTCAAGCTGGCTTTAGCCGGGAGGATAGGCTT | |
| | GAGCAGGCCAAACTCTTCTGCCGGACACTTGAGGACATCCTGGCA | |
| | GATGCCCCTGAGTCTCAGAACAACTGCCGCCTCATTGCCTACCAG | |
| | GAACCTGCAGATGACAGCAGCTTCTCGCTGTCCCAGGAGGTTCTC | |
| | CGGCACCTGCGGCAGGAGGAAAAGGAAGAGGTTACTGTGGGCAGC | |
| | TTGAAGACCTCAGCGGTGCCCAGTACCTCCACGATGTCCCAAGAG | |
| | CCTGAGCTCCTCATCAGTGGAATGGAAAAGCCCCTCCCTCTCCGC | |
| | ACGGATTTCTCTTGA | |

### Plasmids, Vectors, Lipid Nanoparticles

Provided are plasmids comprising a nucleic acid described herein. In some aspects, a plasmid comprises elements for replication in a cell. In some aspects, a plasmid comprises regulatory elements for replication in a bacterial cell. In some aspects, a plasmid comprises regulatory elements for replication in an insect cell.

Also provided are vectors comprising a nucleic acid described herein. In some aspects, a vector is a viral vector. In some aspects, a viral vector is selected from an adenoviral vector, a lentiviral vector and an adeno-associated virus (AAV) vector.

In some aspects, a vector comprises a nucleic acid described herein and sequences, e.g., ITR sequence, of any AAV serotype, such as AAV1, AAV2, AAV3, AAV4, AAV5, AAV6, AAV7, AAV8, AAV9, AAV10, AAV11, AAV12, AAV13, AAVrh10, AAVrh74, AAV2i8, Anc80L65, MyoAAV or a combination of serotypes. In some aspects, a vector, e.g., an AAV vector, comprises an AAV ITR from any AAV serotype. In some aspects, a vector, e.g., an AAV vector is encapsidated within an AAV capsid. In some aspects, a vector, e.g., an AAV vector is encapsidated within an AAV capsid to form a pseudotyped AAV particle, such that the genome, e.g., the AAV vector is of an AAV serotype distinct from the capsid in which it is encapsidated. For example, an AAV vector of serotype AAV2 may be encapsidated within a capsid of serotype AAV9.

In some aspects, an AAV vector comprises a modification, e.g., an insertion, a deletion and/or a substitution of at least one nucleotides of the nucleic acid sequence of the vector.

In some aspects, an AAV vector comprises a single-stranded DNA and comprises an insertion to increase the size of the vector genome. For example, wild-type AAV genomes are approximately 4.7 kilobases (kb) in length and recombinant AAV genomes are typically limited to approximately the same length. See, e.g., Wu, et al., Mol Ther. (2010) 18 (1): 80-86.

In some aspects, an AAV vector comprises an AAV genome having a length greater than a length of a corresponding wild-type AAV genome without substantial negative impacts on AAV genome rescue, replication, and/or packaging.

In some aspects, a vector comprises an AAV genome that has a length of about 4 kilobases (kb), 4.2 kb, 4.3 kb, 4.4 kb, 4.5 kb, 4.6 kb, 4.7 kb, 4.8 kb, 4.9 kb, 5 kb, 5.1 kb, 5.2 kb, 5.3 kb, 5.4 kb, 5.5 kb, 5.6 kb, 5.7 kb, 5.8 kb, 5.9 kb, or about 6 kb. It should be understood that an AAV genome described herein can be of any size or size range within the kb values described herein (e.g., an AAV genome can be 4-6 kb in length, or any length therein between).

In some aspects, an AAV vector comprises a double-stranded DNA, e.g., a self-complementary DNA, and comprises an insertion to increase the size of the vector genome. For example, a wild-type self-complementary AAV genome is approximately 2.4 kilobases (kb) in length and recombinant AAV genomes are typically limited to approximately the same length.

In some aspects, an AAV vector comprises a self-complementary AAV genome having a length greater than a length of a corresponding wild-type self-complementary AAV genome without substantial negative impacts on AAV genome rescue, replication, and/or packaging.

In some aspects, a vector comprises an AAV genome that has a length of about 2.4 kilobases (kb), 2.5 kb, 2.6 kb, 2.7 kb, 2.8 kb, 2.9 kb, or 3 kb in length. It should be understood that an AAV genome described herein can be of any size or size range within the kb values described herein (e.g., a self-complementary AAV genome can be 2.4-3 kb in length, or any length therein between).

In some aspects, a vector, e.g., an AAV vector comprises a STING allele and one or more regulatory elements, such as regulatory elements operably linked to the STING allele. In some aspects, a vector, e.g., an AAV vector comprises a STING allele operably linked to a promoter located between two ITRs, a 5′ ITR and a 3′ ITR. In some aspects, a vector, e.g., an AAV vector comprising a STING allele comprises a regulatory element that is located upstream of or 5′ relative to the STING allele. In some aspects, a vector, e.g., an AAV vector comprising a STING allele comprises a regulatory element that is located downstream of or 3′ relative to the STING allele. In some aspects, a vector, e.g., an AAV vector comprising a STING allele comprises a regulatory element that is located upstream of or 5′ relative to the STING allele and a regulatory element that is located downstream or 3′ relative to the STING allele. In some aspects, a regulatory element is selected from a promoter, enhancer, silencer, insulator, response element, initiation site, termination signal, and ribosome binding site.

In some aspects, a vector, e.g., an AAV vector further comprises a marker gene. In some aspects, a marker gene is, e.g., a gene encoding a green fluorescent protein. In some aspects, a marker gene encodes a detectable molecule, e.g., a molecule that can be visualized (e.g., using a naked eye, under a microscope, or using a light detection device such as a camera). In some aspects, a detectable molecule is a fluorescent molecule, a bioluminescent molecule, or a molecule that provides color (e.g., β-galactosidase, β-lactamase, β-glucuronidase, or spheroidenone). In some aspects, a detectable molecule is a fluorescent, bioluminescent or enzymatic protein or functional peptide or polypeptide thereof. In some aspects, fluorescent protein is a blue fluorescent protein, a cyan fluorescent protein, a green fluorescent protein, a yellow fluorescent protein, an orange fluorescent protein, a red fluorescent protein, or a functional peptide or polypeptide thereof. In some aspects, a detectable molecule is a bioluminescent protein or a functional peptide or polypeptide thereof. Non-limiting examples of bioluminescent proteins are firefly luciferase, click-beetle luciferase, Renilla luciferase, and luciferase from Oplophorus gracilirostris. In some aspects, a detectable molecule may be any polypeptide or protein that can be detected using methods known in the art. Non-limiting methods of detection are fluorescence imaging, luminescent imaging, bright field imaging, and include imaging facilitated by immunofluorescence or immunohisto-chemical staining.

In some aspects, a nucleic acid or a vector, e.g., an AAV vector described herein is comprised in a delivery vehicle such as a liposome, nanocapsule, microparticle, microsphere, lipid particle, vesicle, and the like. In some aspects, a nucleic acid or a vector, e.g., an AAV vector is formulated for delivery either encapsulated in a lipid particle, a liposome, a vesicle, a nanosphere, or a nanoparticle.

### Viral Particles

According to some aspects, provided herein are viral particles that comprise any of the nucleic acids and/or vectors described herein. In some aspects, a viral particle is selected from an adenoviral particle, a lentiviral particle and an AAV particle. The term “AAV particle,” as used herein refers to a supramolecular assembly of 60 individual capsid protein subunits forming a non-enveloped T-1 icosahedral lattice capable of protecting, e.g., a single-stranded DNA genome. For example, a mature AAV2 particle is approximately 20 nm in diameter, and its capsid is formed from three structural capsid proteins VP1, VP2, and VP3, with molecular masses of about 87, 73, and 62 kDa, respectively, in a ratio of about 1:1:18. The 60 capsid proteins are arranged in an anti-parallel β-strand barreloid arrangement, resulting in a defined tropism and a high resistance to degradation. In some aspects, an AAV particle comprises a capsid formed from three structural capsid proteins VP1, VP2, and VP3 of an AAV1, AAV2, AAV3, AAV4, AAV5, AAV6, AAV7, AAV8, AAV9, AAV10, AAV11, AAV12, AAV13, AAVrh10, AAVrh74, AAV218, Anc80L65, MyoAAV or combinations thereof. In some embodiments, the AAV particle is an AAV5 particle and is used, for example, to deliver its cargo to the subject's lungs. In some embodiments, the AAV particle is an AAV8 particle and is used, for example, to deliver its cargo to the subject's brain. In some embodiments, the AAV particle is an AAV9 particle and is used, for example, to deliver its cargo to the subject's liver. In some embodiments, the AAV particle is an AAVrh8 particle and is used, for example, to deliver its cargo to the subject's spleen. In some embodiments, the AAV particle is an AAVrh10 particle and is used, for example, to deliver its cargo to the subject's heart. In some embodiments, an AAV5 particle comprises a cap

In some aspects, an AAV particle comprises a capsid encapsidating a nucleic acid or a vector, e.g. an AAV vector comprising a STING allele. In some aspects, a nucleic acid encapsidated within an AAV capsid to generate an AAV particle comprises a nucleic acid and/or a vector, e.g., an AAV vector described herein. In some aspects, an AAV particle described herein comprises a capsid protein comprising one or more mutations, e.g., one or more amino acid substitutions.

In some aspects, an AAV particle described herein is replicative. A replicative AAV particle is capable of replicating within a host cell (e.g., a host cell within a subject or a host cell in culture). In some aspects, an AAV particle described herein is non-replicating. A non-replicating AAV particle is not capable of replicating within a host cell (e.g., a host cell within a subject or a host cell in culture) but can infect the host and deliver a nucleic acid or vector, e.g., an AAV vector comprising a STING allele to the host cell. In some aspects, an AAV particle described herein is capable of facilitating stable integration of a genetic component, e.g., a STING allele into the genome of a host cell. In some aspects, an AAV particle described herein is not capable of facilitating integration of a genetic component into the genome of a host cell.

In some aspects, an AAV vector comprises two inverted terminal repeats (ITRs) adjacent to the ends of a sequence comprising a nucleic acid or vector, e.g., an AAV vector comprising a STING allele operably linked to a promoter. In some aspects, an AAV vector is a single-stranded DNA vector. In some aspects, an AAV particle described herein comprises one single-stranded DNA. In some aspects, an AAV vector is a double-stranded DNA vector. In some aspects, an AAV particle described herein comprises two complementary DNA strands, forming a self-complementary AAV (scAAV).

An AAV particle described herein may comprise a capsid protein of any AAV serotype (e.g., AAV1, AAV2, AAV3, AAV4, AAV5, AAV6, AAV7, AAV8, AAV9, AAV10, AAV11, AAV12, AAV13, AAVrh10, AAVrh74, AAV218, Anc80L65, MyoAAV or a combination thereof), including any derivative (including non-naturally occurring variants of a serotype) or pseudotype. AAV particles and AAV particle derivatives/AAV particle pseudotypes, and methods of producing AAV particles and derivatives/pseudotypes are known in the art (see, e.g., Mol. Ther. 2012 April; 20 (4): 699-708. The AAV vector toolkit: poised at the clinical crossroads. Asokan A, Schaffer D V, Samulski R J.). In some aspects, the AAV particle is a pseudotyped AAV particle, which comprises a vector, e.g., an AAV vector comprising ITRs from one serotype (e.g., AAV2) and a capsid comprised of capsid proteins derived from another serotype (i.e., a serotype other than AAV2 like, e.g., an AAV9). Methods for producing and using rAAV vectors and pseudotyped rAAV vectors are known in the art (see, e.g., Duan et al., J. Virol., 75:7662-7671 (2001); Halbert et al., J. Virol., 74:1524-1532 (2000); Zolotukhin et al., Methods, 28:158-167 (2002); and Auricchio et al., Hum. Molec. Genet., 10:3075-3081 (2001)).

In some aspects, a vector, e.g., an AAV vector comprises a first and/or a second AAV ITR and the first and/or second AAV ITR is selected from AAV1, AAV2, AAV3, AAV5, AAV6, AAV7, AAV8, AAV9, AAV10, AAVrh10, AAVrh74, AAV218, Anc80L65, and MyoAAV, or fragments or variants thereof.

In some aspects, an AAV particle comprises a vector, e.g., an AAV vector comprising an ITR from one serotype and a capsid comprised of capsid proteins derived from the same serotype. In some aspects, an AAV particle comprises a vector, e.g., an AAV vector comprising an ITR from an AAV2 serotype and a capsid from an AAV2 serotype. In some aspects, an AAV particle comprises a vector, e.g., an AAV vector comprising an ITR from an AAV2 serotype and a capsid, e.g., from an AAV1, AAV2, AAV3, AAV5, AAV6, AAV7, AAV8, AAV9, AAV10, AAV12, AAV13, AAVrh10, AAVrh74, AAV218, Anc80L65, and MyoAAV or fragments or variants thereof.

In some aspects, an AAV particle comprises a vector, e.g. an AAV vector comprising an ITR from an AAV9 serotype and a capsid, e.g., from an AAV1, AAV2, AAV3, AAV5, AAV6, AAV7, AAV8, AAV9, AAV10, AAV12, AAV13, AAVrh10, AAVrh74, AAV2i8, Anc80L65, and MyoAAV, or fragments or variants thereof.

Examples of amino acid sequences of AAV capsid proteins of various serotypes are provided as SEQ ID NOs: 10-23.

In some aspects, an AAV particle comprises a capsid protein having one or more modifications characterized by an amino acid substitution. In some aspects, an AAV particle described herein comprises a capsid protein having one or more modifications compared to an AAV2 capsid protein of SEQ ID NO: 11. In some aspects, an AAV particle described herein comprises a capsid protein having one or more modifications compared to an AAV9 capsid protein of SEQ ID NO: 18. In some aspects, an AAV particle described herein comprises a capsid protein having one or more modifications compared to an AAV capsid protein of SEQ ID NO: 10-23. In some aspects, an AAV particle described herein comprises a capsid protein of SEQ ID NO: 10-23.

Corresponding positions in capsid proteins having different baseline amino acid sequences can be determined by methods known in the art, such as by constructing structural alignments of the amino acid sequences of capsid proteins of various AAV serotypes and identifying corresponding amino acids. A “corresponding” amino acid to be substituted is one which is at the corresponding position, and may have the same amino acid identity (i.e., the amino acid at the corresponding position in the second capsid protein sequence is the same as the amino acid in the reference capsid protein sequence), or may be an amino acid with similar properties (e.g., similar hydrophobicity, size, charge, etc.) as the amino acid in the reference capsid protein. The different capsid proteins VP1, VP2, and VP3 are defined according to numbering of the full-length VP1 protein. In some aspects, an AAV2 VP1 capsid protein is defined by amino acids 1-736 of SEQ ID NO: 11. In some aspects, an AAV9 VP1 capsid protein is defined by amino acids 1-736 of SEQ ID NO: 18. Numbering of AAV capsid proteins is provided according to the VP1 sequence.

In some aspects, a capsid protein is a chimeric capsid protein comprising an N-terminal sequence of a capsid protein of one AAV serotype and a C-terminal sequence of a capsid protein of another AAV serotype. For example, in some aspects, a chimeric capsid protein comprises an N-terminal sequence of an AAV2 capsid protein and a C-terminal sequence of an AAV9 capsid protein. In some aspects, a chimeric capsid protein comprises an N-terminal sequence of an AAV2 capsid protein and a C-terminal sequence of an AAV1, AAV3, AAV5, AAV6, AAV7, AAV8, AAV9, AAV10, AAV12, AAV13, AAVrh10, AAVrh74, AAV2i8, Anc80L65, and MyoAAV capsid protein. In some aspects, a chimeric capsid protein comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 35, 36 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more amino acids of an N-terminal sequence of a capsid protein of one AAV serotype and the remaining amino acid sequence to prepare a full-length capsid protein sequence is a capsid protein sequence of another AAV serotype.

In some aspects, a nucleic acid comprises a sequence that encodes a capsid protein described herein (e.g., AAV1, AAV2, AAV3, AAV5, AAV6, AAV7, AAV8, AAV9, AAV10, AAV12, AAV13, AAVrh10, AAVrh74, AAV2i8, Anc80L65, and MyoAAV). A sequence encoding a capsid protein can be determined by one of ordinary skill in the art by known methods. A nucleic acid encoding a capsid protein may comprise a promoter or other regulatory sequence operably linked to the coding sequence. A nucleic acid encoding a capsid protein may be in the form of a plasmid, an mRNA, or another nucleic acid capable of being used by enzymes or machinery of a host cell to produce a capsid protein. Nucleic acids encoding capsid proteins as provided herein can be used to make AAV particles described herein that can be used for delivering a gene to a cell. Methods of making AAV particles are known in the art. For example, see Scientific Reports volume 9, Article number: 13601 (2019); Methods Mol Biol. 2012; 798:267-284. Example sequences of nucleic acids encoding capsid proteins are provided as SEQ ID NOs: 24-34.

### Transgene Expression

According to some aspects, a STING protein described herein is expressed in an immune cell or a non-immune cell. In some aspects, an immune cell is a lymphocyte, a natural killer cell, a monocyte, a macrophage, a dendritic cell, a neutrophile, a granulocyte, a basophile, an eosinophile, a mast cell, a hematopoietic stem cell, or a Kupffer cell. In some aspects, a non-immune cell is a lung cell, a liver cell, a muscle cell, a cardiac cell, a kidney cell, or a joint cell. In some aspects, a non-immune lung cell is an alveolar epithelial cell, a bronchial epithelial cell, a respiratory epithelial cell, a pulmonary endothelial cell, a pulmonary smooth muscle cell, a pulmonary adventitial fibroblast, a mesenchymal cell, or a pulmonary lymphoid cell. In some aspects, a non-immune liver cell is a hepatocyte, a cholangiocyte, a biliary epithelial cell, a liver sinusoidal endothelial cell, or a liver progenitor cell. In some aspects, a non-immune cardiac cell is a cardiomyocyte, a cardiac endothelial cell, a cardiac smooth muscle cell, a cardiac myofibroblast. In some aspects, a non-immune joint cell is a chondrocyte, a synovial fibroblast, a synovial adipocyte, an osteoblast, or an osteoclast.

In some aspects, a STING protein encoded by a nucleic acid, a vector, and/or a viral particle described herein comprising a R293Q (Q) STING allele, a G230A-R293Q (AQ) STING allele, a R71H-G230A-R293Q (HAQ) STING allele, or a C148R STING allele is specifically expressed in an immune cell following administration of the nucleic acid, vector and/or viral particle to a subject. In some aspects, a STING protein encoded by a nucleic acid, a vector, and/or a viral particle described herein comprising a R293Q STING allele, a G230A-R293Q STING allele, a R71H-G230A-R293Q STING allele, or a C148R STING allele is not expressed in a non-immune cell following administration of the nucleic acid, vector and/or viral particle to the subject. In some aspects, a STING protein encoded by a nucleic acid, a vector, and/or a viral particle described herein comprising a R293Q STING allele, a G230A-R293Q STING allele, a R71H-G230A-R293Q STING allele, or a C148R STING allele is expressed in a non-immune cell following administration of the nucleic acid, vector and/or viral particle to the subject.

In some aspects, a nucleic acid, vector and/or viral particle described herein comprising a R293Q STING allele, a G230A-R293Q STING allele, a R71H-G230A-R293Q STING allele, or a C148R STING allele result in a greater copy number per nucleus of a R293Q STING allele, a G230A-R293Q STING allele, a R71H-G230A-R293Q STING allele, or a C148R STING allele in an immune cell compared to a non-immune cell, e.g., a liver cell following administration of a nucleic acid, vector, and/or viral particle described herein to a subject.

In some aspects, a nucleic acid, vector and/or viral particle as described herein result in a higher expression of a STING protein encoded by a nucleic acid comprising a R293Q STING allele, a G230A-R293Q STING allele, a R71H-G230A-R293Q STING allele, or a C148R STING allele in an immune cell compared to a non-immune cell on a per nucleic acid vector copy number basis (e.g., STING protein expression in a cell, when normalized to the total amount of STING encoding nucleic acid in the cell, is altered). Relative protein expression levels can be determined, for example, by measuring expression, e.g., of a STING protein in a cell by methods known in the art following contacting the cell with a nucleic acid, vector and/or viral particle comprising a polynucleotide encoding, e.g., a STING protein.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in an immune cell is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in a non-immune cell, e.g., a liver cell following administration of a nucleic acid, vector, and/or viral particle to a subject.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in an immune cell is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, at least 25-fold higher, at least 30-fold higher, at least 35-fold higher, at least 40-fold higher, at least 45-fold higher, at least 50-fold higher, at least 55-fold higher, at least 60-fold higher, at least 65-fold higher, at least 70-fold higher, at least 75-fold higher, at least 80-fold higher, at least 85-fold higher, at least 90-fold higher, at least 100-fold higher or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in a non-immune cell, e.g., a liver cell following administration of a nucleic acid, vector, and/or viral particle to a subject.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in a CD4+ T cell is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in a non-CD4 T immune cell, e.g., a CD8+ T cell or B cell following administration of a nucleic acid, vector, and/or viral particle to a subject.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in a CD4+ T cell is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, at least 25-fold higher, at least 30-fold higher, at least 35-fold higher, at least 40-fold higher, at least 45-fold higher, at least 50-fold higher, at least 55-fold higher, at least 60-fold higher, at least 65-fold higher, at least 70-fold higher, at least 75-fold higher, at least 80-fold higher, at least 85-fold higher, at least 90-fold higher, at least 100-fold higher or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in non-CD4+T immune cell, e.g., a CD8+ T cell or B cell following administration of a nucleic acid, vector, and/or viral particle to a subject.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in a CD4⁺CD8⁺ cell is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in a non-CD4⁺CD8⁺ cell following administration of a nucleic acid, vector, and/or viral particle to a subject.

In some aspects, the STING protein expression from a nucleic acid, vector, and/or viral particle as described herein comprising a R293Q STING allele, a G230A-R293Q STING allele, a R71H-G230A-R293Q STING allele, or a C148R STING allele in a CD4⁺CD8⁺ cell is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, at least 25-fold higher, at least 30-fold higher, at least 35-fold higher, at least 40-fold higher, at least 45-fold higher, at least 50-fold higher, at least 55-fold higher, at least 60-fold higher, at least 65-fold higher, at least 70-fold higher, at least 75-fold higher, at least 80-fold higher, at least 85-fold higher, at least 90-fold higher, at least 100-fold higher or more) than the expression of a STING protein from a nucleic acid, vector, and/or viral particle as described herein comprising a R293Q STING allele, a G230A-R293Q STING allele, a R71H-G230A-R293Q STING allele, or a C148R STING allele in a non-CD4⁺CD8⁺ cell following administration of a nucleic acid, vector, and/or viral particle to a subject.

### Compositions

Any one of the nucleic acids, plasmids, vectors, or AAV particles described herein may be comprised within a composition. In some aspects, a composition is a pharmaceutical composition. In some aspects, a pharmaceutical composition comprises a pharmaceutically acceptable carrier or may be comprised within a pharmaceutically-acceptable carrier. The term “carrier” refers to a diluent, adjuvant, excipient, or vehicle with which the nucleic acid, plasmid, vector, or AAV particle is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

In some aspects, a composition comprises at least about 0.1% of the therapeutic agent (e.g., nucleic acid, plasmid, vector, or AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., nucleic acid, plasmid, vector, or AAV particle) in each therapeutically-useful composition may be prepared in such a way that a suitable dosage will be obtained in any given unit dose of the nucleic acid, plasmid, vector, or AAV particle. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

In some aspects, a composition comprises a nucleic acid that comprises a nucleotide sequence for genomic integration. In some aspects, the nucleic acid further comprises flanking 5′ and 3′ homology arms and a gene editing agent. In some aspects, a gene editing agent comprises a programmable nuclease or a nucleotide sequence encoding a programmable nuclease. In some aspects, the programmable nuclease is an RNA-guided nuclease, a transcription Activator-Like Effector Nuclease (TALEN), or a Zinc Finger Nuclease (ZNF). In some aspects, the editing agent further comprises a guide RNA (gRNA). In some aspects, the gRNA targets the site for genomic integration to which the 5′ homology arm and the 3′ homology arm are homologous.

In some aspects, a nucleic acid comprising a nucleotide sequence for genomic integration comprises an inverted terminal repeat (ITR). In some aspects, a nucleic acid comprising a nucleotide sequence for genomic integration comprises an AAV ITR. In some aspects, a nucleic acid comprises a sequence for genomic integration into a safe harbor. In some aspects, a nucleic acid comprises a sequence for genomic integration into an AAVS1. In some aspects, a composition comprises a nucleic acid comprising a nucleotide sequence for genomic integration comprising an AAV ITR. In some aspects, a composition comprising a nucleic acid comprising an AAV ITR further comprises a replication protein or a nucleotide sequence encoding a replication protein. In some aspects, a replication protein is an AAV Rep78, AAV Rep68, AAV Rep52, or a AAV Rep42.

Pharmaceutical compositions as disclosed herein, e.g., comprising a nucleic acid, plasmid, vector, AAV particle or a composition comprising a nucleic acid comprising a R293Q STING allele, G230A-R293Q STING allele, R71H-G230A-R293Q STING allele, or C148R STING allele can be prepared by one of ordinary skill in the art by known methods.

### Methods of Contacting a Cell

According to some aspects, methods of contacting a cell with a nucleic acid, plasmid, vector, or AAV particle are provided herein. Methods of contacting a cell may comprise, for example, contacting a cell in a culture with a composition comprising a nucleic acid, plasmid, vector, or AAV particle. In some aspects, contacting a cell comprises adding a composition comprising a nucleic acid, plasmid, vector, or AAV particle to a supernatant of a cell culture (e.g., a cell culture on a tissue culture plate or dish) or mixing a composition comprising a nucleic acid, plasmid, vector, or AAV particle with a cell culture (e.g., a suspension cell culture). In some aspects, contacting a cell comprises mixing a composition comprising a nucleic acid, plasmid, vector, or AAV particle with another solution, such as a cell culture media, and incubating a cell with the mixture.

In some aspects, contacting a cell with a nucleic acid, plasmid, vector, or AAV particle comprises administering a composition comprising a nucleic acid, plasmid, vector, or AAV particle to a subject. In some aspects, contacting a cell with a nucleic acid, plasmid, vector, or AAV particle comprises administering a composition comprising a nucleic acid, plasmid, vector, or AAV particle to a device in which the cell is located. In some aspects, contacting a cell comprises injecting a composition comprising a nucleic acid, plasmid, vector, or AAV particle into a subject in which the cell is located. In some aspects, contacting a cell comprises administering a composition comprising a nucleic acid, plasmid, vector, or AAV particle directly to a cell, or into or substantially adjacent to a cell or a tissue of a subject in which the cell is present.

### Methods of Treatment

In some aspects, “administering” or “administration” means providing a material to a subject in a manner that is pharmacologically useful. In some aspects, a nucleic acid, plasmid. vector, or AAV particle is administered to a subject enterally. In some aspects, an enteral administration of a nucleic acid, plasmid, vector, or AAV particle is oral. In some aspects, a nucleic acid, plasmid, vector, or AAV particle is administered to the subject parenterally. In some aspects, a nucleic acid, plasmid, vector, or AAV particle is administered to a subject by subcutaneous, intravenously, intraperitoneally, intramuscular, intraosseous, intraarticular, intracerebro-ventricular, intraocular, intravitreal, subretinal, intrathecal, intracisternal injection and/or infusion or by inhalation, topically, or by direct injection to one or more cells, tissues, or organs.

In some aspects, a composition comprising a nucleic acid, plasmid, vector, or AAV particle is administered to a subject to treat a disease or condition. The term “treat,” “treating.” or “treatment,” as used herein, refers to reduce the frequency or severity of at least one sign or symptom of a disease or disorder experienced by a subject. In some embodiments, “treat.” “treating.” or “treatment” refers to delaying the onset of a disease or disorder (e.g., delaying the onset of Alzheimer's disease).

In some aspects, a composition comprising a nucleic acid, plasmid, vector, or AAV particle is administered to a subject to assist with the treatment of a disease or condition.

In some aspects, provided is a method of suppressing a gain-of-function mutant STING allele in a subject, the method comprising administering to the subject a therapeutically effective amount of a nucleic acid described herein, a composition described herein and/or an AAV particle described herein. In some aspects, the method further comprises determining the presence of a gain-of-function STING allele in a subject. In some aspects, provided is a method of suppressing a gain-of-function mutant STING allele in a subject, the method comprising administering to a subject that has been determined to have a gain-of-function STING allele a therapeutically effective amount of a nucleic acid described herein, a composition described herein and/or an AAV particle described herein.

In some aspects, provided is a method of treating STING-associated vasculopathy with onset in infancy (SAVI), the method comprising administering to the subject a therapeutically effective amount of a nucleic acid described herein, a composition described herein and/or an AAV particle described herein. In some aspects, the method further comprises determining the presence of SAVI in a subject. In some aspects, provided is a method of treating SAVI in a subject, the method comprising administering to a subject that has been determined to have SAVI a therapeutically effective amount of a nucleic acid described herein, a composition described herein and/or an AAV particle described herein.

In some aspects, provided is a method of suppressing a STING-mediated inflammatory response in a subject, the method comprising administering to the subject a therapeutically effective amount of a nucleic acid described herein, a composition described herein and/or an AAV particle described herein. In some aspects, the method further comprises determining the presence of a STING-mediated inflammatory response in a subject. In some aspects, provided is a method of treating a STING-mediated inflammatory response in a subject, the method comprising administering to a subject that has been determined to have a STING-mediated inflammatory response a therapeutically effective amount of a nucleic acid described herein, a composition described herein and/or an AAV particle described herein.

In some aspects, provided is a method of treating a STING-mediated inflammatory disease or disorder in a subject, the method comprising administering to the subject a therapeutically effective amount of a nucleic acid described herein, a composition described herein and/or an AAV particle described herein. In some aspects, the method further comprises determining the presence of a STING-mediated inflammatory disease or disorder in a subject. In some aspects, provided is a method of treating a STING-mediated inflammatory disease or disorder in a subject, the method comprising administering to a subject that has been determined to have a STING-mediated inflammatory disease or disorder a therapeutically effective amount of a nucleic acid described herein, a composition described herein and/or an AAV particle described herein.

The term “STING-mediated inflammatory disease or disorder,” as used herein, refers to any disease or disorder that is characterized by an activation of STING. In some aspects, a STING-mediated inflammatory disease or disorder is an autoimmune disease, including but not limited to, systemic lupus erythematosus, Sjoergen syndrome, type 1 diabetes, Hashimoto thyroiditis, rheumatoid arthritis, celiac disease, inflammatory bowel disease, ulcerative colitis, Crohn's disease, Graves' disease, multiple sclerosis, myasthenia gravis, psoriasis, dermatomyositis, scleroderma, Guillain-Barre syndrome, vasculitis, vitiligo, hemolytic anemia, Addison's disease, pernicious anemia, and/or autoimmune hepatitis. In some aspects, a STING-mediated inflammatory disease or disorder includes, but is not limited to, asthma, eczema, ankylosing spondylitis, gout, and/or Schonlein-Henoch purpura. In some aspects, a STING-mediated inflammatory disease or disorder includes, but is not limited to, a neurodegenerative disease, such as Alzheimer's disease, Parkinson's Disease, and/or amyotrophic lateral sclerosis.

The nucleic acid, composition, and/or AAV particle described herein are typically administered to a subject in an “effective amount”. The term “effective amount” as used herein refers to an amount capable of producing a desirable result. The desirable result will depend upon the disease or disorder to be treated or prevented by the administration of a nucleic acid, plasmid, vector, AAV particle, or composition described herein. For example, an effective amount of a composition comprising a nucleic acid, plasmid, vector, or AAV particle may be an amount of the nucleic acid, plasmid, vector, or AAV particle that is capable of transferring a nucleic acid comprising a R293Q STING allele, a G230A-R293Q STING allele, a R71H-G230A-R293Q (HAQ) STING allele, or a C148R STING allele to a host organ, tissue, or cell, such that the present of the transferred STING allele prevents or treats a STING-mediated inflammatory response or STING-mediated inflammatory disease or disorder.

In some aspects, a composition comprising a nucleic acid, plasmid, vector, or AAV particle is administered to a subject in a therapeutically effective amount. The term “therapeutically effective amount.” as used herein, refers to an amount that is capable of treating a disease or disorder, e.g., STING-mediated disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some aspects, a composition comprising AAV particles comprising a nucleic acid, plasmid, or vector described herein is administered at a concentration of AAV particles to a subject ranging from about 10³ to about 10¹⁵ particles/ml, about 10⁶ to about 10¹⁴ particles/ml or any values therebetween for either range, such as for example, about 10³, about 10⁴, about 10⁵, about 10⁶, about 10⁷, about 10⁸, about 10⁹, about 10¹⁰, about 10¹¹, about 10¹², about 10¹³, about 10¹⁴, or about 10¹⁵ particles/ml. In some aspects, AAV particles of a higher concentration than about 10¹³ particles/ml are administered. In some aspects, the concentration of AAV particles administered to a subject may be on the order ranging from about 10³ to about 10¹⁵ vector genomes (vgs)/ml or about 10⁶ to about 10¹⁴ vgs/ml, or any values therebetween for either range (e.g., about 10³, about 10⁴, about 10⁵, about 10⁶, about 10⁷, about 10⁸, about 10⁹, about 10¹⁰, about 10¹¹, 10¹², about 10¹³, about 10¹⁴, or about 10¹⁵ vgs/ml). In some aspects, AAV particles of higher concentration than 10¹³ vgs/ml are administered. The AAV particles can be administered as a single dose, or divided into two or more administrations as may be required to achieve therapy of the particular disease or disorder being treated. In some aspects. 0.0001 ml to 10 ml are delivered to a subject. In some aspects, the number of AAV particles administered to a subject may be on the order ranging from about 10³ to about 10¹⁵ vgs/kg body mass, or about 10⁶-about 10¹⁴ vgs/kg body mass of the subject, or any values therebetween (e.g., about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵ vgs/kg). In some aspects, the dose of AAV particles administered to a subject may be on the order ranging from about 10¹⁰-about 10¹² vgs/kg, from about 10¹²-about 10¹³ vgs/kg, or from about 10¹²-about 10¹⁴ vgs/kg. In some aspects, the volume of a composition delivered to a subject (e.g., via one or more routes of administration as described herein) is 0.0001 ml to 10 ml.

In some aspects, a cell disclosed herein is a cell isolated or derived from a subject. In some aspects, a cell disclosed herein is a cell within a subject. In some aspects, a cell is a mammalian cell (e.g., a cell isolated or derived from a mammal). In some aspects, a cell is a human cell. In some aspects, a cell is isolated or derived from a particular tissue of a subject. In some aspects, a cell is an immune cell. In some aspects, a cell is a hematopoietic cell. In some aspects, a cell is a lymphocyte. In some aspects, a cell is a monocyte. In some aspects, a cell is a T cell. In some aspects, a cell is a CD4⁺ T cell. In some aspects, a cell is a CD8⁺ T cell. In some aspects, a cell is a B cell. In some aspects, a cell is a neutrophil. In some aspects, a cell is a natural killer cell. In some aspects, a cell is a cell of a mucosa associated lymphoid tissue. In some aspects, a cell is a cell of a gut-associated lymphoid tissue.

In some aspects, a cell is an endothelial cell. In some aspects, a cell is a lung epithelial cell. In some aspects, a cell is a Kupffer cell. In some aspects, a cell is a microglia cell. In some aspects, a cell is a synovial cell.

In some aspects, a cell is in vitro. In some aspects, a cell is ex vivo. In some aspects, a cell is in vivo. In some aspects, a cell is within a subject (e.g., within a tissue or organ of a subject). In some aspects, a cell is a primary cell. In some aspects, a cell is from a cell line (e.g., an immortalized cell line). In some aspects a cell is a cancer cell or an immortalized cell.

In some aspects, a composition disclosed herein (e.g., comprising an AAV particle) is administered to a subject once. In some aspects, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

### Subjects

Aspects of the disclosure relate to methods for use with a subject, such as a mammal, e.g., a human subject. In some aspects, a method comprises contacting a host cell in situ in a subject, or a host cell derived from a subject (e.g., ex vivo or in vitro) with a composition described herein. In some aspects, a mammal is a non-human primate. Non-limiting examples of non-human primates include macaques (e.g., cynomolgus or rhesus macaques), marmosets, tamarins, spider monkeys, owl monkeys, vervet monkeys, squirrel monkeys, baboons, gorillas, chimpanzees, and orangutans. In some aspects, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some aspects, the subject has or is suspected of having a disease or disorder that may be treated with a composition described herein. In some aspects, methods described herein include administration of a nucleic acid described herein, a composition described herein and/or an AAV particle described herein to a subject that may have one or more of the following diseases or disorders: systemic lupus erythematosus, Sjoergen syndrome, type 1 diabetes, Hashimoto thyroiditis, rheumatoid arthritis, celiac disease, inflammatory bowel disease, ulcerative colitis, Crohn's disease, Graves' disease, multiple sclerosis, myasthenia gravis, psoriasis, dermatomyositis, scleroderma, Guillain-Barre syndrome, vasculitis, vitiligo, hemolytic anemia, Addison's disease, pernicious anemia, and/or autoimmune hepatitis. In some aspects, a subject may have one or more of the following STING-mediated inflammatory diseases or disorders: asthma, eczema, ankylosing spondylitis, gout, and/or Schonlein-Henoch purpura.

In some aspects, the subject has, or is suspected of having STING-associated vasculopathy with onset in infancy (SAVI).

In some aspects, the subject is an infant (e.g., less than 2 years of age, less than 1 year of age, less than 6 months of age). In some aspects, the subject is 2-8 years of age, 9-14 years of age, 15-18 years of age, 19-25 years of age, 26-50 years of age, 50-60 years of age, 61-80 years of age, or over 80 years of age. In some embodiments, a subject is an African-American human. In some embodiments, the subject is a non-Hispanic white human.

In some aspects, a subject affected by an inflammatory disease comprises a reduced number and/or reduced activation of regulatory T (Treg) cells. In some aspects, methods described herein include administration of a composition described herein and/or an AAV particle described herein to a subject affected by an inflammatory disease that is characterized by a reduced number and/or reduced activity of regulatory T (Treg) cells.

### EXAMPLES

### Example 1. Methods

### Experimental Design.

The study was designed to reveal (i) the in vivo significance of the type I IFNs-independent. STING-dependent cell death function and (ii) the interplay between common STING alleles HAQ, AQ and the rare, gain-of-function SAVI STING mutation; the driver for the inflammatory SAVI disease. Mouse splenocytes, primary human lung cells, human THP-1 cells and HAQ, AQ. SAVI knock-in mice were used to establish the in vivo significance and human relevance. All the repeats were biological replications that involve the same experimental procedures on different mice. When comparing samples from different groups, samples from each group were analyzed in concert, thereby preventing any biases that might arise from analyzing individual treatments on different days. All experiments were repeated at least twice.

### Mice.

WT/SAVI(N153S) mice were purchased from The Jackson Laboratory. HAQ, AQ mice were previously generated in the lab (Patel S., et al., J Immunol 198:776-787, 2017; Mansouri S., et al., J Immunol 209:2114-213233, 2022.). The Q293 mice were generated by Cyagen Biosciences. Briefly, the linearized targeting vector was transfected into JM8A3.N1 C57BL/6N embryonic stem cells. A positive embryonic stem clone was subjected to the generation of chimera mice by injection using C57BL/6J blastocysts as the host. Successful germline transmission was confirmed by PCR sequencing. The heterozygous mice were bred to Actin-flpase mice [The Jackson Laboratory, B6.Cg-Tg (ACTFLPe) 9205Dym/J] to remove the neo gene and make the Q293 knock-in mouse. Age- and gender-matched mice (2-6 month-old, both male and female) were used for indicated experiments. WT/SAVI (male)×WT/HAQ (female), WT/SAVI (male)×WT/AQ (female) breeders were set up to generate HAQ/SAVI, AQ/SAVI mice. Mice were housed at 22° C. under a 12-h light-dark cycle with ad libitum access to water and a chow diet (3.1 kcal/g. Teklad 2018, Envigo, Sommerset, NJ) and bred under pathogen-free conditions. Littermates of the same sex were randomly assigned to experimental groups.

### Reagents

Recombinant human IFNβ (R&D, cat no. 8499-IF-010/CF), diABZI (Invivogen, cat no. 2138299-34-8), 2′3′-cGAMP (Invivogen, cat no. tlrl-nacga23-02), DMXAA (Invivogen, cat no. tlrl-dmx), H151 (Invivogen, cat no. inh-h151), RpRpSSCDA (Biolog, cat no. C118), THP1-Dual™ KO-STING Cells (Invivogen, cat no. thpd-kostg) and Mouse IFN-Beta ELISA Kit (PBI, cat no. 42400) were purchased. All other chemical inhibitors were obtained from Selleckchem. Mouse TNF alpha ELISA Ready Set Go. (eBioscience, cat no. 88-7324).

### Histology

Lungs and livers were fixed in 10% formalin, paraffin-embedded, and cut into 4-μm sections. Lung, liver sections were then stained for hematoxylin-eosin. Briefly, tissue sections were immersed in Harris Hematoxylin for 10 seconds, then washed with tap water. Cleared sections were re-immersed in EOSIN stain for ˜30 seconds. The sections were washed with tap water until clear, then dehydrated in ascending alcohol solutions (50%, 70%, 80%, 95% twice, 100% twice). Afterwards, the sections were cleared with xylene (3-4×). The sections were mounted on glass slide with permount organic mounting medium for visualization.

### Lung Function

Pulmonary function was evaluated using an isolated, buffer-perfused mouse lung apparatus (Hugo Sachs Elektronik, March-Huggstetten, Germany), as previously described (Cai J. et al., J Heart Lung Transplant 39:1476-1490, 2020). Briefly, mice were anesthetized with ketamine and xylazine and a tracheostomy was performed, and animals were ventilated with room air at 100 breaths/min at a tidal volume of 7 μl/g body weight with a positive end-expiratory pressure of 2 cm H2O using a pressure-controlled ventilator (Hugo Sachs Elektronik, March-Huggstetten, Germany).

### Isolation of Lung Cells

Cells were isolated from the lung as previously described (Mansouri S., et al., Mucosal Immunol 13:595-608 68, 2020). The lungs were perfused with ice-cold PBS and PBS was removed. Lungs were digested in DMEM containing 200 μg/ml DNase I (Roche, 10104159001), 25 μg/ml Liberase™ (Roche, 05401119001) at 37° C. for 2 hours. Red blood cells were then lysed and a single cell suspension was prepared by filtering through a 70-μm cell strainer.

### BMDM Activation

Bone marrow derived macrophages (BMDMs) were induced from mouse bone marrow cells cultured in RPMI 1640 (cat #11965; Invitrogen) with 10% FBS, 2 mM L-glutamine, 1 mM sodium pyruvate, 10 mM HEPES buffer, 1% nonessential amino acids, 50 mM 2-ME, 1% Pen/Strep, with 20 ng/ml M-CSF (Kingfisher, RP0407M) for 10 days (Patel S., et al., J Immunol 198:776-787, 2017). STING agonists were added into the culture (no transfection or membrane permeabilization).

### Flow Cytometry

Single cell suspensions were stained with fluorescent-dye-conjugated antibodies in PBS containing 2% FBS and 1 mM EDTA. Surface stains were performed at 4° C. for 20 min. For intracellular cytokine or transcription factor staining of murine and human cells, cells were fixed and permeabilized with the Foxp3 staining buffer set (eBioscience, cat no 00-5523-00). Cells were washed and stained with surface markers. Cells were then fixed and permeabilized (eBioscience, cat no. 00-5523-00) for intracellular cytokine stain. Data were acquired on a BD LSRFortessa and analyzed using the FlowJo software package (FlowJo, LLC). Cell sorting was performed on the BD FACSAriaIII Flow Cytometer and Cell Sorter.

### Human Lung Explants

Healthy human donor lungs were surgically removed postmortem, perfused, small pieces were cut from the right middle and lower lobes for research purpose and stored in cold Perfadex® at 4° C. for no more than 12 hrs before processing. Explanted lungs from emphysema lung transplant patients were stored in cold Perfadex® at 4° C. for no more than 12 hrs before processing.

### Statistical Analysis

To gain statistical power, about three to four mice/groups were used to characterize lung immunity. Ten mice/group were used to monitor animal health. The statistical justification for group size was calculated using the SAS program to calculate the animal numbers. The analysis was carried out using a standard error of 0.5 for immunological assays, and a power of 0.9. All data are expressed as means±SEM. Statistical significance was evaluated using Prism 9.0 software. Comparisons between two groups were analyzed by performing an unpaired Student's t test. Comparisons between more than two groups were analyzed by performing a one-way analysis of variance (ANOVA) with Tukey's multiple comparisons test.

### Example 2—STING Activation Kills Mouse Spleen CD4, CD8, and CD19 B Cells Ex Vivo

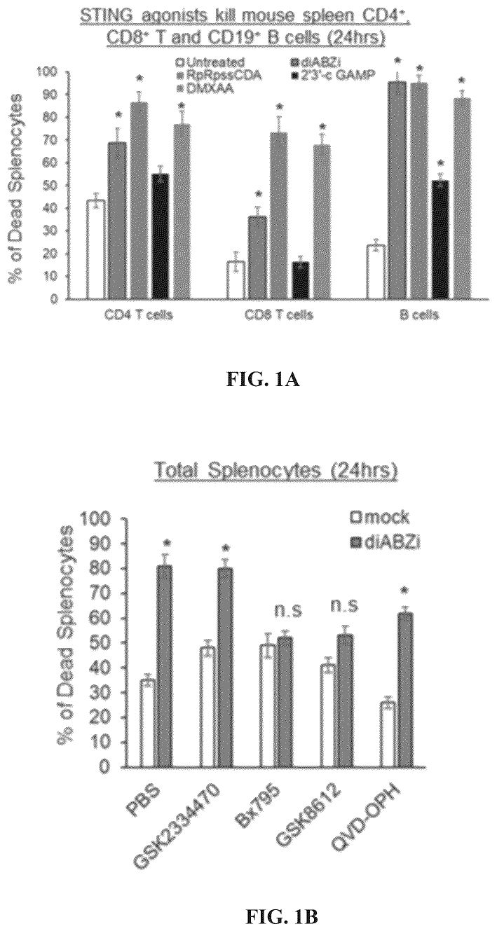

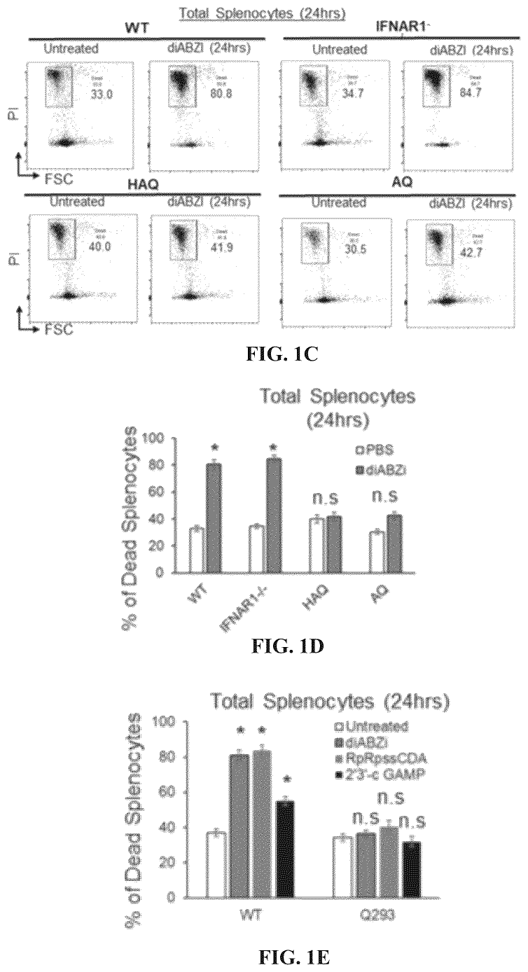

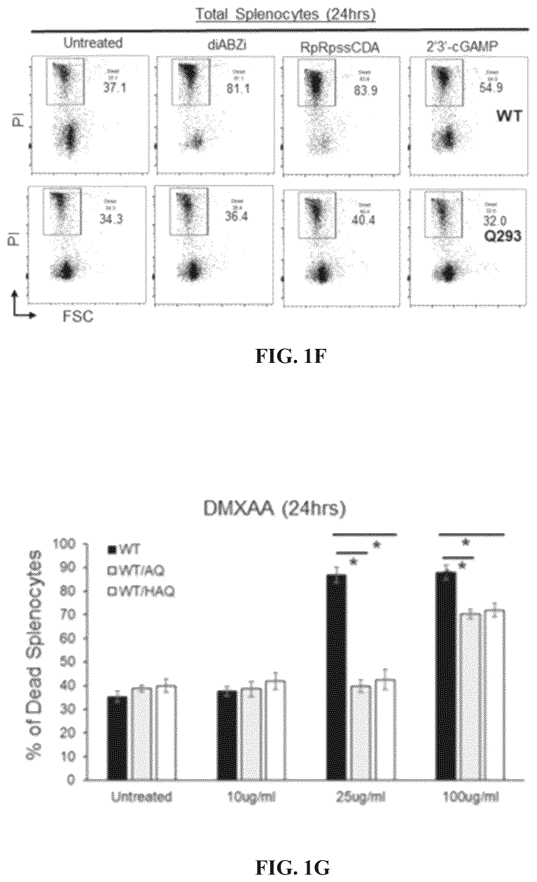

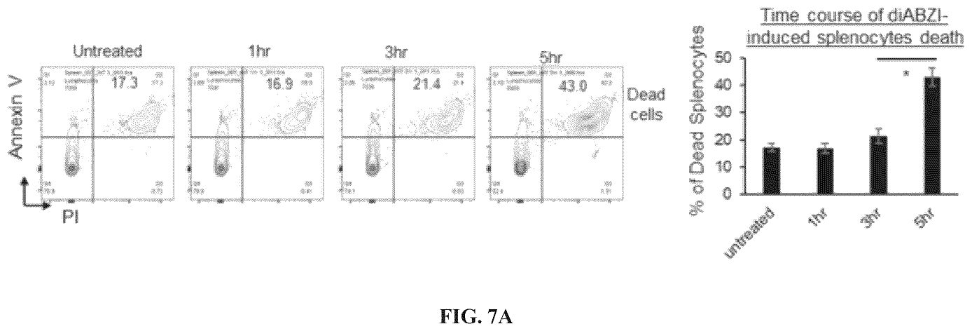

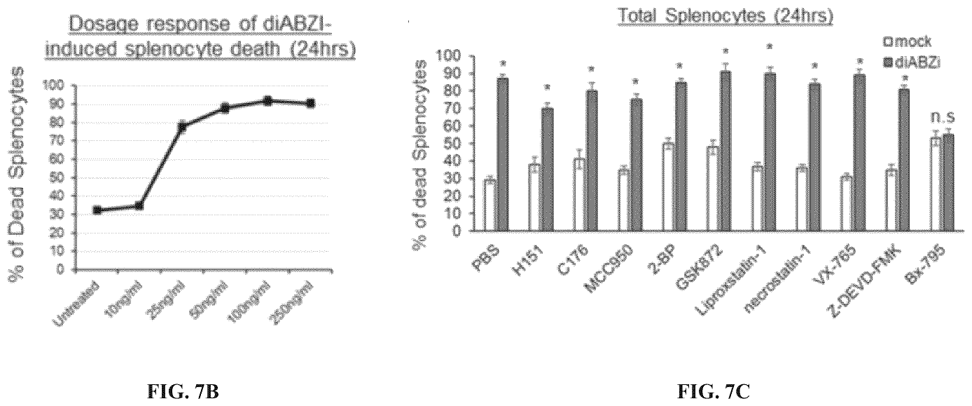

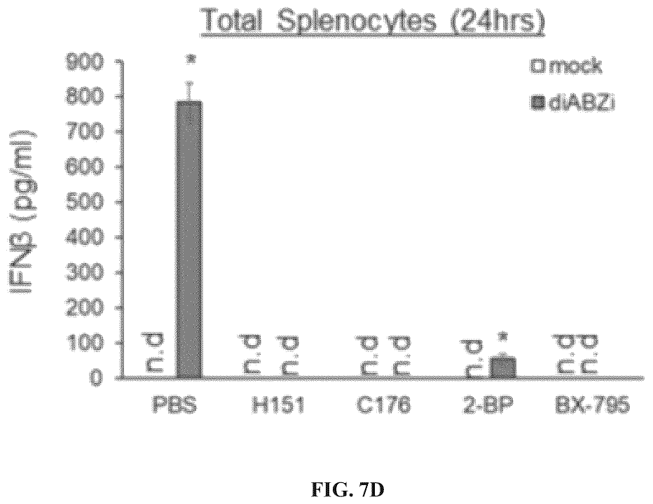

The synthetic non-CDNs STING agonist diABZI (Ramanjulu J. M. et al., Nature 564:439-443, 2018) was used to induce lymphocyte death because diABZI induces cell death without the need for lipid transfection or detergent for cell permeabilization (Kabelitz D., et al., Sci Rep 12, 2022; Messaoud-Nacer Y., et al., Cell Death Dis 1342, 2022). diABZI has been used in a clinical trial (NCT05514717). C57BL/6N splenocytes were treated directly (no transfection) with diABZI (100 ng/ml), RpRpssCDA (5 μg/ml), 2′3′-cGAMP (10 μg/ml), or DMXAA (25 μg/ml) for 24 hrs in culture. CD4 T cell, CD8 T cell and CD19 B cell death was determined by PI staining (FIG. 1A). Splenocytes from C57BL/6N mice were treated with diABZI in culture, and cell death was determined by Annexin V and Propidium Iodide stain. Splenocyte cell death could be detected as early as 5 hrs post diABZI treatment (FIG. 7A). Dosage responses showed that ˜25 ng/ml diABZI could kill 70% of splenocytes (FIG. 7B). Similarly, STING agonists DMXAA, and synthetic CDNs RpRpssCDA, killed mouse spleen CD4, CD8 T cells, and CD19 B cells (FIG. 1A). Thus, STING activation readily induced mouse lymphocyte death ex vivo.

### Example 3—TBK1 Activation is Required for STING Mediated Mouse Spleen Cell Death Ex Vivo

STING activation can lead to apoptosis, pyroptosis, necroptosis, or ferroptosis (Kuhl N. et al., EMBO Rep 24, 2023; Jin L., et al., Mol Cell Biol 28:5014-5026, 2008; Gulen M. F., et al., Sci Rep 12, 2017; Murthy A. M. V., et al., Cell Death Differ 27:2989-3003, 2020; Li C., et al., Cell Dev Biol 9, 2021; Song C. et al. Sci Rep 1225, 2022; Messaoud-Nacer Y., et al., Cell Death Dis 13, 2022; Tang C. H., et al., Cancer Res 76:2137-2152, 2016). Mouse splenocytes were treated with apoptosis, pyroptosis, necroptosis inhibitors, STING inhibitors H-151, C-176, palmitoylation inhibitor 2-bromopalmitate (2-BP), followed by diABZI stimulation. Splenocytes from C57BL/6N mice were pre-treated with GSK2334470 (1.25 μM), GSK8612 (2.5 μM), Bx-795 (0.5 μM), QVD-OPH (25 μM) for 2 hrs and diABZI (100 ng/ml) was added in culture for another 24 hrs before dead cells were determined by PI staining. Inhibitors for NLRP3 (MCC950), RIPK1 (Necrostatin-1), RIPK3 (GSK872), Caspase-1 (VX-795), Caspase-3 (Z-DEVD-FMK), Caspase 1,3,8,9 (Q-VD-Oph), ferroptosis (liproxstatin-1) did not affect diABZI-induced splenocyte cell death ex vivo (FIG. 1B and FIG. 7B-7C). The STING inhibitors H-151, C-176, and 2-BP also could not prevent diABZI-induced cell death (FIG. 7C) though they inhibited diABZI-induced IFNβ production (FIG. 7D). Instead, the TBK1 inhibitor BX-795 abolished diABZI-induced splenocyte death (FIG. 7C). BX-795 is a multi-kinase inhibitor including 3-phosphoinositide-dependent protein kinase 1 (PDK1) and TBK1 (IC50s=6 and 11 nM, respectively). However, the treatment of PDK1 inhibitor GSK2334470 (IC50=10 nM) did not prevent diABZI-induced splenocyte death (FIG. 7B). In contrast, GSK8612, a highly potent and selective inhibitor for TBK1, prevented diABZI-induced splenocyte death (FIG. 1B). These data showed that TBK1 activation was critical for STING-mediated lymphocyte cell death ex vivo.

### Example 4—HAQ, AQ, Q293 STING Knock-In Mouse Splenocytes are Resistant to STING-mediated Cell Death Ex Vivo

HAQ and AQ are common human TMEM173 alleles (Jin L., Genes Immun 12:263-269, 2011; Patel S., et al., J Immunol 198:776-787, 2017; Mansouri S., et al., J Immunol 209:2114-2132, 2022). Previously, it was reported that HAQ knock-in mice are defective in CDNs-induced immune responses, while CDN responses in AQ knock-in mice were similar to WT mice (Mansouri S., et al., J Immunol 209:2114-2132, 2022).

Splenocytes from HAQ and AQ mice treated with diABZI ex vivo were surprisingly found to be resistant to diABZI-induced cell death (FIG. 1C-1D). In comparison, IFNAR1^{-/-} splenocytes were killed by diABZI, confirming that STING-mediated lymphocyte death is type I IFN-independent (FIG. 1C-1D) (Kuhl N. et al. EMBO Rep 24, 2023; Gulen M. F., et al., Nat Commun 8, 2017; Murthy A. M. V., et al., Cell Death Differ 27:2989-3003, 2020). HAQ and AQ share the common A230 and Q293 residues changes. Thus, a Q293 TMEM173 knock-in mouse was generated. Notably, the Q293 splenocytes were resistant to STING agonists 2′3′-cGAMP, RpRpssCDA, and diABZI-induced cell death (FIGS. 1E-1F). Thus, the residue 293 of STING was determined to be critical for STING's cell death function.

### Example 5—WT/HAQ, WT/AQ Mouse Splenocytes were Partially Resistant to STING-Mediated Cell Death Ex Vivo

WT/HAQ (34.3%), not WT/WT (22.0%), is the most common human STING genotype in East Asians, while WT/AQ (28.2%) is the 2nd most common STING genotype in Africans (Patel S., et al, J Immunol 198:776-787, 2017). WT/HAQ and WT/AQ mice were generated and the splenocytes were treated mouse STING agonist DMXAA. WT/HAQ and WT/AQ splenocytes were protected from 25 μg/ml DMXAA-induced cell death (FIG. 1G). 100 μg/ml DMXAA could kill WT/HAQ and WT/AQ splenocytes albeit less than WT/WT cells (FIG. 1G). Thus, the HAQ and AQ alleles are dominant and impact STING activation even in heterozygosity.

### Example 6—STING Activation Kills Primary Human CD4 T Cells, but not CD8 T or CD19 B Cells

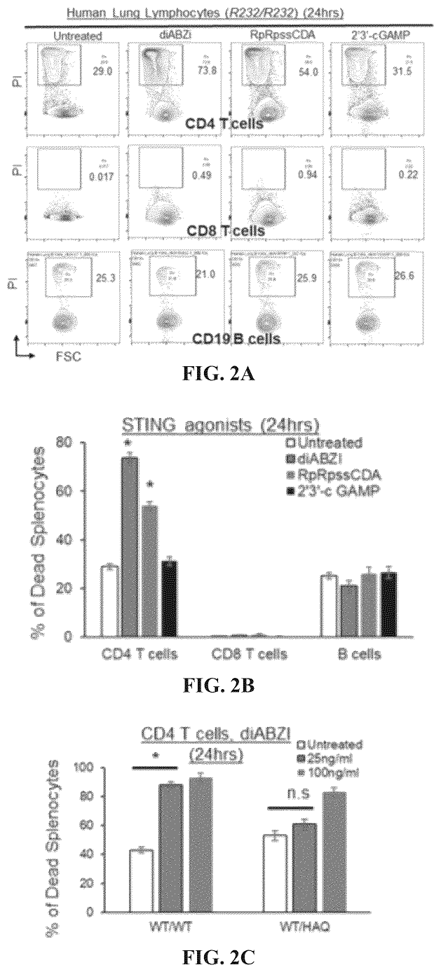

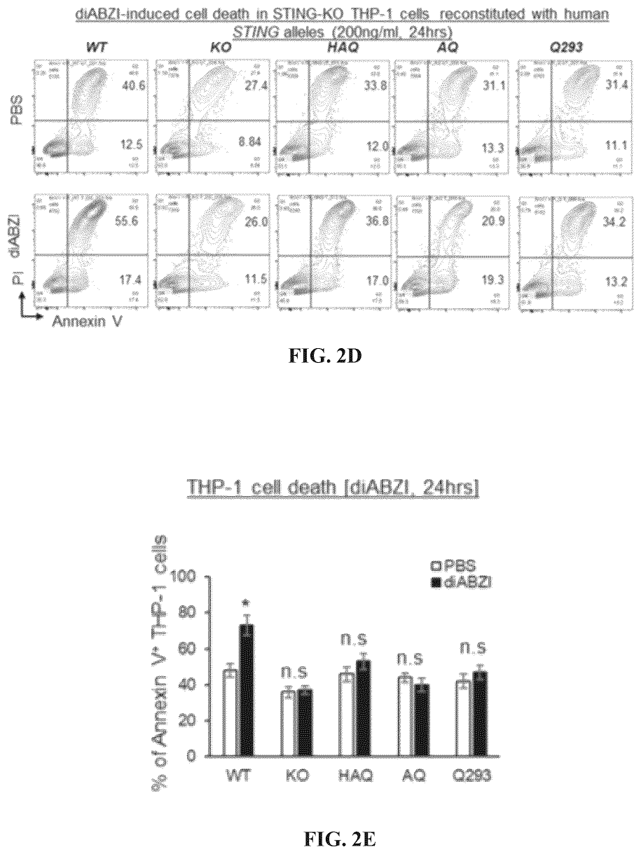

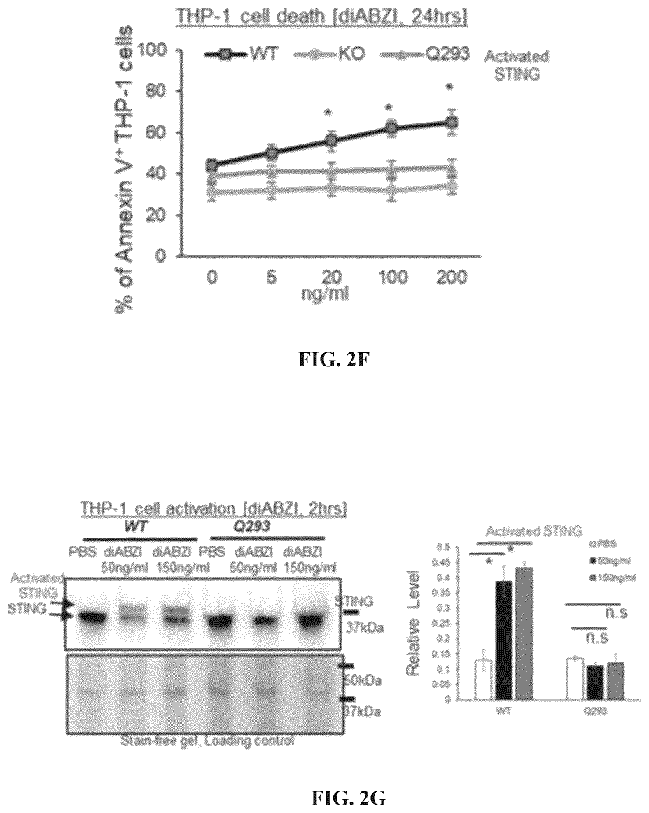

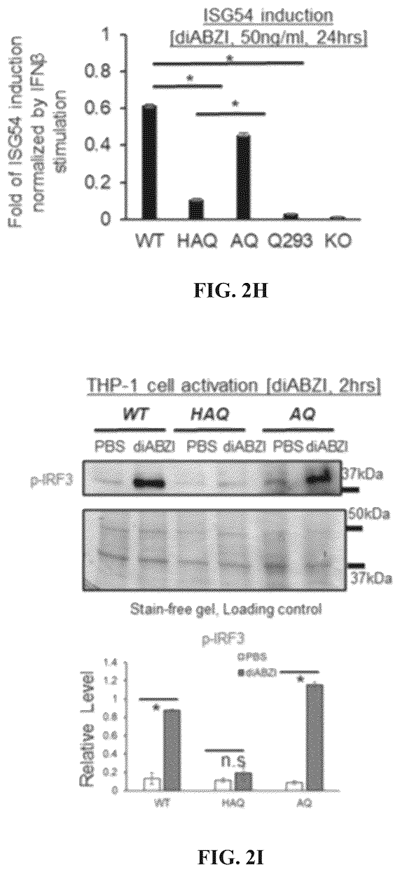

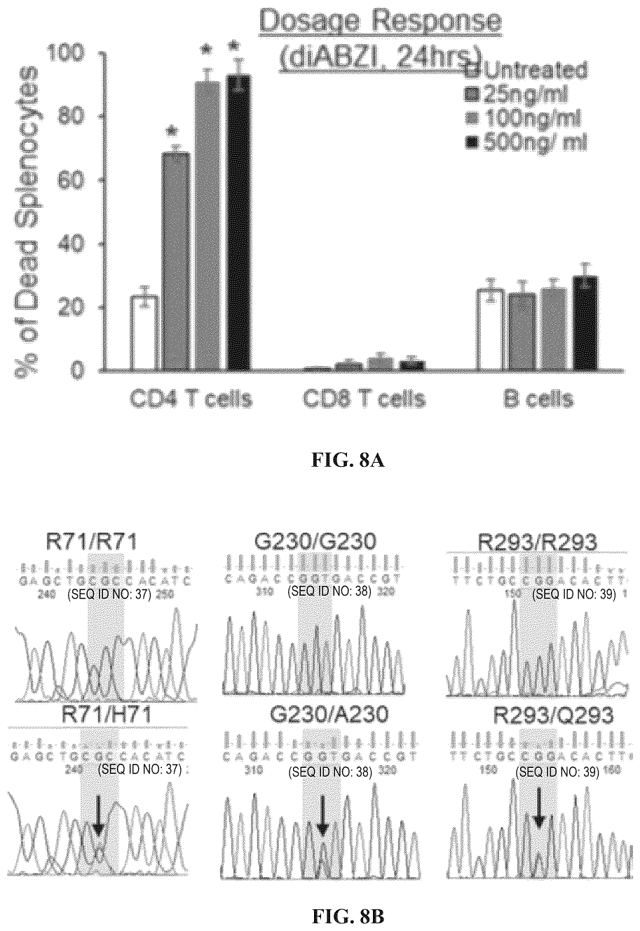

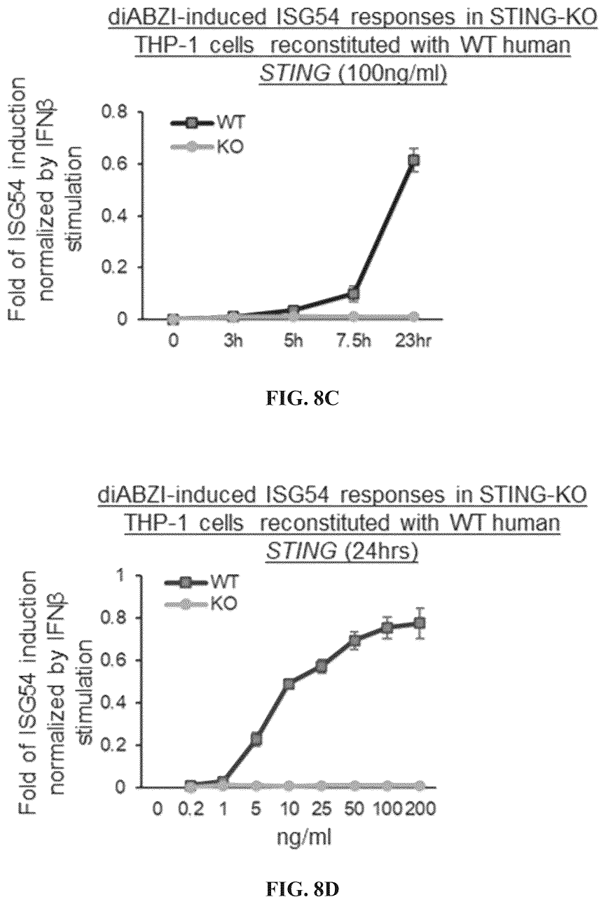

STING agonist-based clinical trials in humans have been disappointing (NCT02675439, NCT03010176, NCT05514717) (Meric-Bernstam F. et al. Clin Cancer Res 29:110-121, 2023; Meric-Bernstam F. et al. Clin Cancer Res 28:677-6884, 2022). The human TMEM173 gene might have undergone natural selection during the out-of-Africa migration sensitive to evolutionary pressure (Mansouri S., et al., J Immunol 209:2114-2132, 2022). Thus, STING-mediated death in primary human lymphocytes was investigated. Human explant lung cells from WT (R232)/WT (R232) donors were treated with STING agonists 2′3-c GAMP, RpRpssCDA, diABZI for 24 hrs in culture. Lymphocyte cell death was determined by Propidium Iodide staining. Different from mouse lymphocytes, diABZI and RpRpssCDA killed human CD4 T but not CD8 T or CD19 B cells (FIG. 2A-2B). Human CD8 T and CD19 B cells were resistant to 500 ng/ml diABZI-induced cell death (FIG. 8A).

### Example 7—WT/HAQ Human CD4 T Cells are Resistant to Low-Dose of diABZI-Induced Cell Death

WT/HAQ mouse splenocytes were resistant to low dose diABZI-induced cell death. To examine primary human T cells, lung explants were obtained from WT/WT and WT/HAQ individuals (FIG. 8B) Total lung cells from a WT/HAQ (2 individuals) and a WT/WT (3 individuals) were treated with diABZi (25, 100 ng/ml) for 24 h and cell death in CD4 T cells was determined by PI staining. 25 ng/ml diABZI killed WT/WT, but not WT/HAQ, human lung CD4 T cells (FIG. 2C).

### Example 8—diABZI Induced Cell Death in STING-KO Human THP-1 Cells Reconstituted with WT Human STING (R232) but not HAQ, AQ or Q293 Human STING Allele

To further determine cell death influenced by human TMEM173 alleles HAQ, AQ and Q293, a STING-KO THP-1 cell line (Invivogen, cat no. thpd-kostg) was used because STING agonist induced type I IFNs and cell death in STING-KO THP-1 cells expressing WT human STING 31 (FIGS. 8C-8D). Stable THP-1 STING-KO lines expressing HAQ, AQ. WT or Q293 TMEM173 alleles were generated, and cells were treated with diABZI (200 ng/ml) in culture for 24 hrs. diABZI killed THP-1 STING-KO lines expressing WT but not HAQ, AQ, or Q293 TMEM173 allele (FIGS. 2D-2E). No cell death was induced in the Q293 THP-1 cells stimulated by 20 to 200 ng/ml of diABZI (FIG. 2F). diABZI also did not induce STING activation in Q293 THP-1 cells; STING activation was detected by anti-STING antibody (Proteintech, #19851-1-AP) (FIG. 2G). Notably, 50 ng/ml diABZI induced p-IRF3 activation (CST, Ser396, clone 4D4G) and type I IFNs in AQ THP-1 cells as detected by ISG-54 reporter luciferase activity determined in cell supernatant and normalized to 10 ng/ml IFNβ-stimulated ISG-54 luciferase activity, but not HAQ THP-1 cells (FIGS. 2H-2I) indicating that the STING-cell death and STING-IRF3-Type I IFNs pathways can be uncoupled.

### Example 9—HAQ and AQ Alleles Rescue the Lymphopenia and Suppress Myeloid Cell Expansion in SAVI(N153S) Mice

The in vivo significance of the STING/MPYS-cell death is unclear. Furthermore, multiple cell death pathways, i.e., apoptosis, necroptosis, pyroptosis, ferroptosis, and PANoptosis, were proposed (Murthy A. M. V., et al., Cell Death Differ 27:2989-3003, 2020; Li C., et al., Front Cell Dev Biol 9, 2021; Song C. et al., Sci Rep 12, 2022). The uncertainty likely results from studies using different cell types (primary cells vs cancer cell lines); species (human vs mouse); STING agonists (cGAMP, which requires cell permeabilization by detergents or lipid transfection, vs diABZi, DMXAA that can directly cross the membrane) (Larkin B., et al., J Immunol 199:397-402, 2017; Cerboni S. et al. J Exp Med 214:1769-1785, 2017; Gulen M. F., et al., Sci Rep 12, 2017; Wu J., et al., J Exp Med 216:867-883, 2019). Importantly, it is not known which mechanism is relevant in vivo causing T cellpenia. To clarify the in vivo significance and mechanisms of STING-mediated cell death, SAVI mice were used.

STING-associated vasculopathy with onset in infancy (SAVI) is an autosomal dominant, inflammatory disease caused by one copy of a gain-of-function STING mutant (Liu Y. et al. N Engl J Med 371:507-518, 2014). CD4 T cellpenia was found in SAVI patients and SAVI mouse models (Liu Y. et al. N Engl J Med 371:507-518, 2014; Luksch H., et al., J Allergy Clin Immunol 144:254-266, 2019). STING activation in SAVI mice is independent of ligands and occurs in vivo. To establish the in vivo significance and mechanism of STING-cell death, HAQ/SAVI(N153S) and AQ/SAVI(N153S) mice were generated.

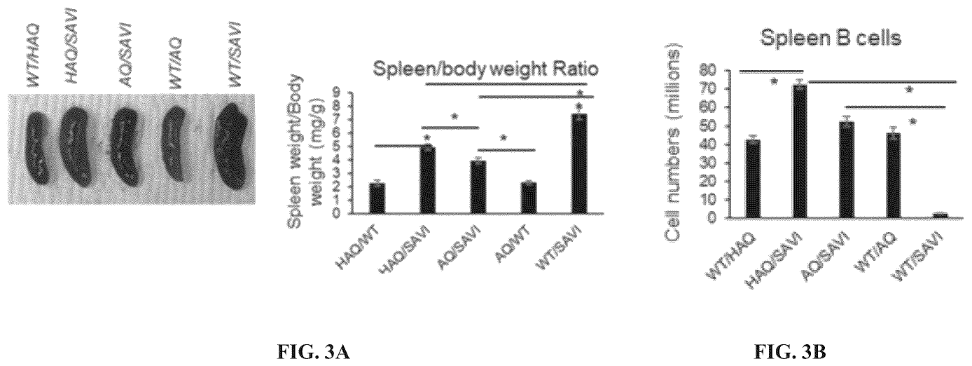

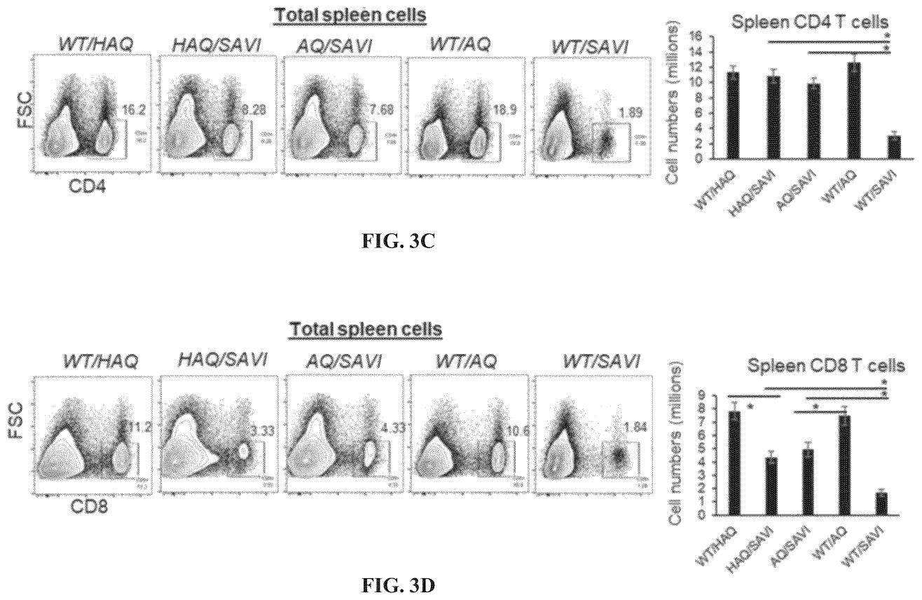

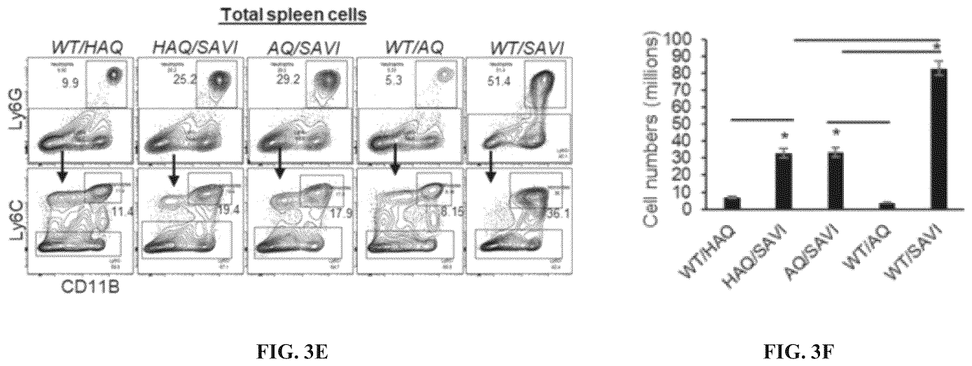

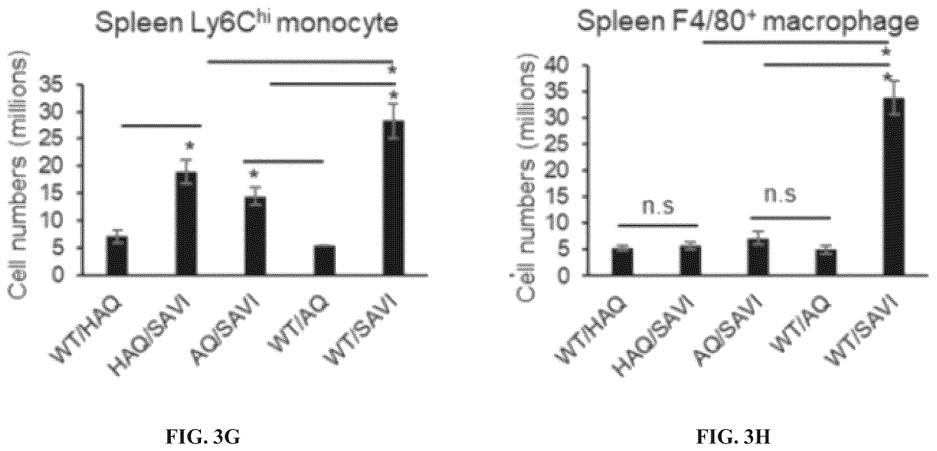

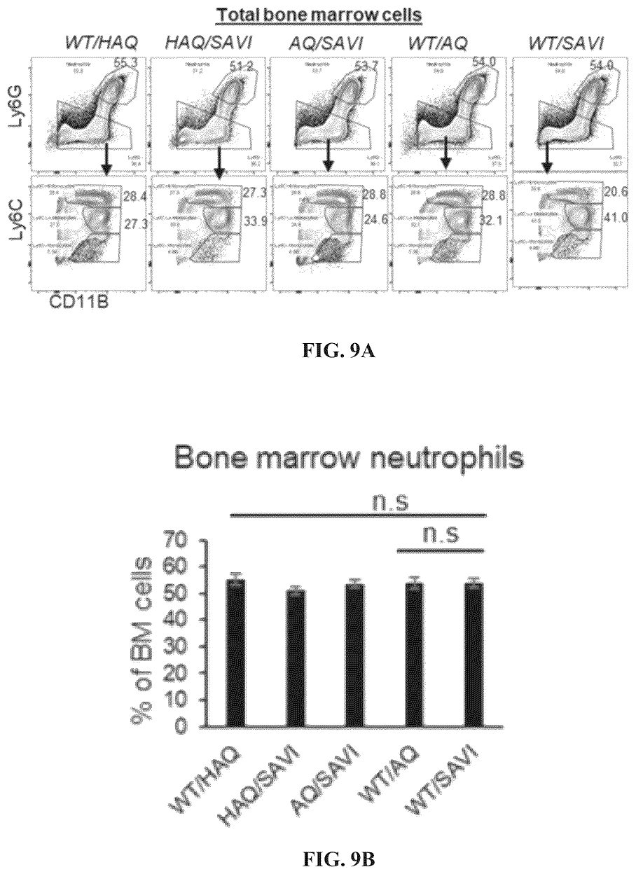

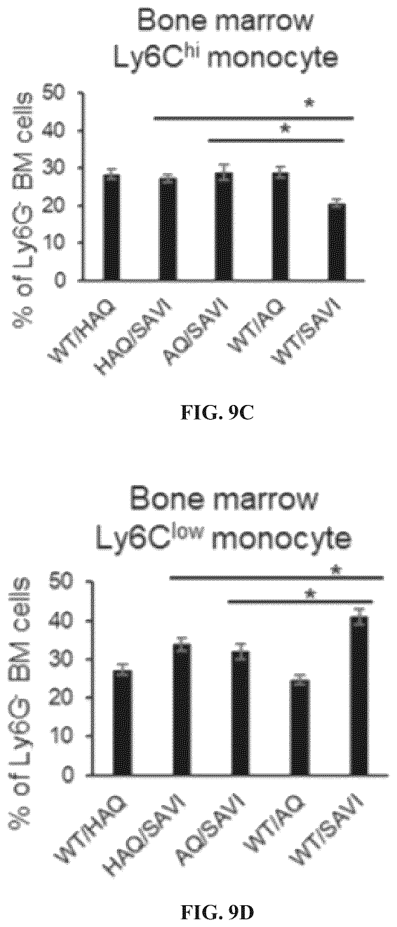

First, HAQ/SAVI(N153S) and AQ/SAVI(N153S) mice had reduced splenomegaly compared to WT/SAVI(N153S) mice though their spleens were still larger than the littermates WT/HAQ and WT/AQ (FIG. 3A-3B). Next, HAQ/SAVI(N153S) and AQ/SAVI(N153S) mice had similar spleen B cells and CD4 T cells numbers as the WT/HAQ, WT/AQ littermates (FIG. 3B-3C). The CD8+ T cells in WT/HAQ and WT/AQ mice were lower than in the WT littermates but much higher than the WT/SAVI(N153S) mice (FIG. 3D). Third, spleen myeloid cell numbers, i.e., neutrophils, Ly6C^{hi} monocytes and F4/80 macrophages, were all reduced by half compared to WT/SAVI(N153S) mice (FIG. 3E-3H). Notably, the HAQ/SAVI(N153S) and AQ/SAVI(N153S) mice also had restored bone marrow monocyte (FIG. 9A-9D). Thus, HAQ and AQ alleles prevented lymphopenia and suppressed myeloid cells expansion in SAVI(N153S) mice.

### Example 10—The HAQ Allele Alleviated and the AQ Allele Prevented SAVI(N153S) Disease in Mice

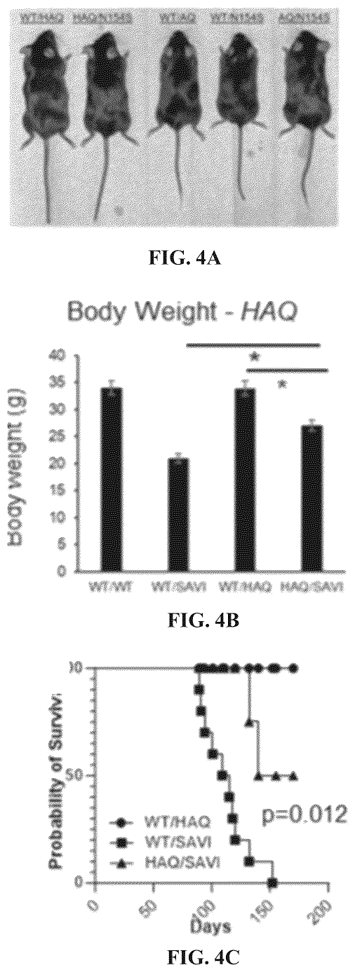

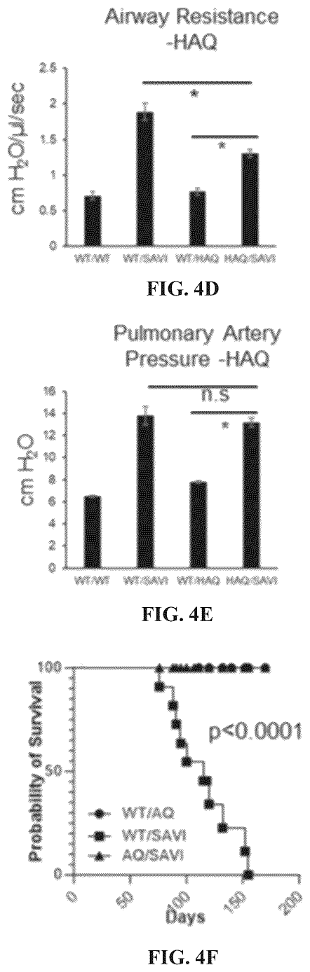

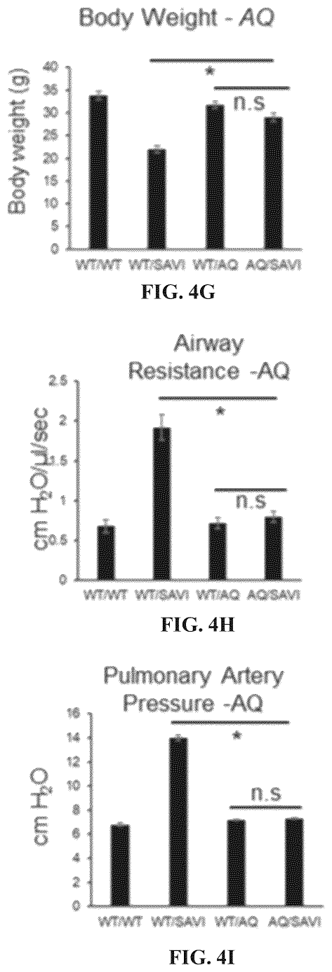

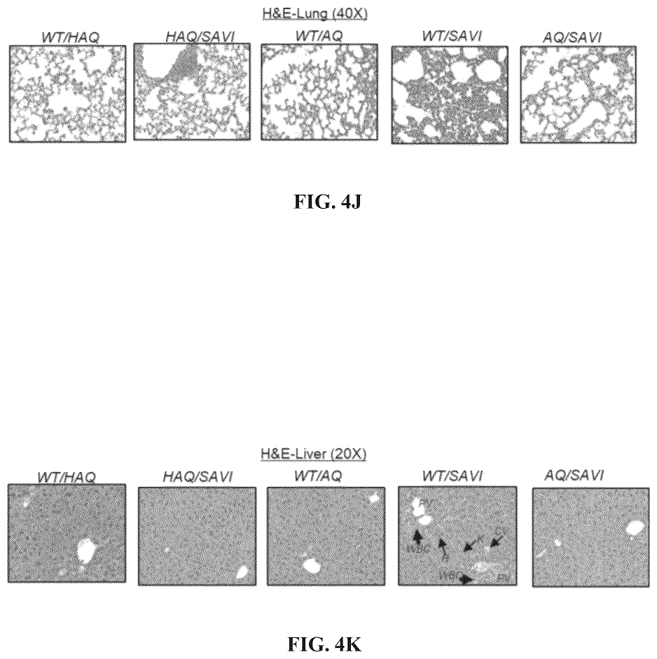

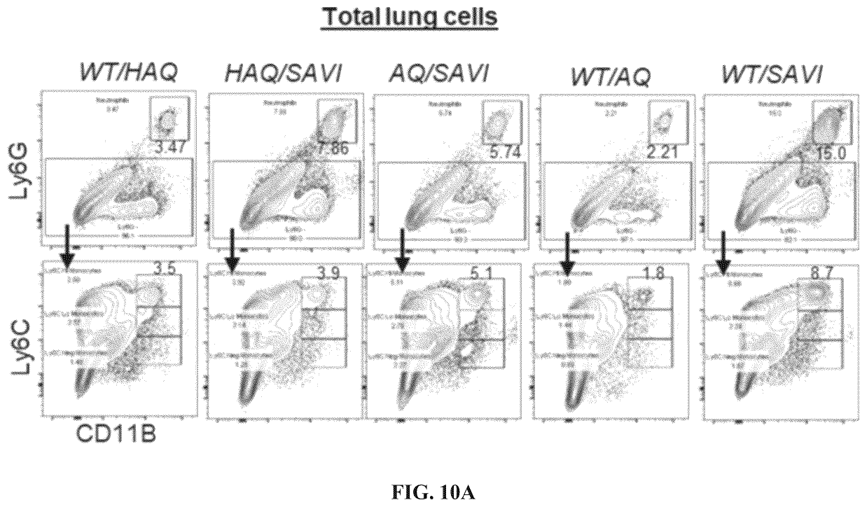

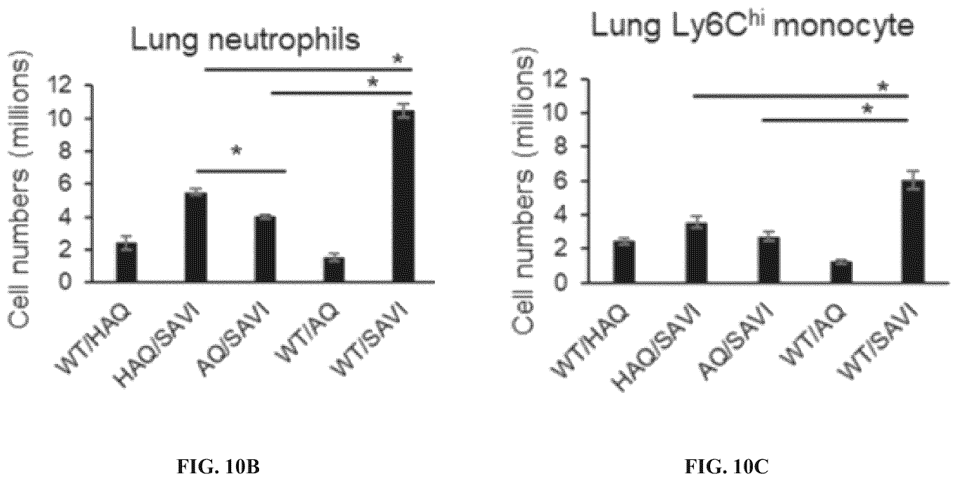

SAVI(N153S) disease is characterized by early onset, failure to thrive (low body weight), persistent lung inflammation, decreased lung function, and young death in humans and mouse models (Liu Y. et al., N Engl J Med 371:507-518, 2014; Fremond M. L. et al., J Allergy Clin Immunol Pract 9:803-818, 2021; Wu J., et al., J Exp Med 216:867-883, 2019; Motwani M., et al., Proc Natl Acad Sci USA 116:7941-7950, 2019). The HAQ/SAVI(N153S) mice weighed more and had an improved lifespan than the WT/SAVI(N153S) mice (FIG. 4A-4B). The lifespan, airway resistance and tissue inflammation (lung, liver) were also improved in HAQ/SAVI(N153S) mice compared to the WT/SAVI(N153S) mice (FIG. 4C, 4D, 4J, 4K, and FIG. 10). However, the pulmonary artery pressure was still elevated in HAQ/SAVI(N153S) mice (FIG. 4E). Remarkably, the AQ/SAVI(N153S) mice had similar body weight and lifespan as the WT/AQ mice (FIG. 4F-4G). The airway resistance, pulmonary artery pressure, and tissue inflammation in AQ/SAVI(N153S) were similar to the WT/AQ littermates (FIGS. 4H-4K, and FIG. 10). Thus, the AQ allele prevented inflammatory SAVI disease in mice.

### Example 11—diABZI Induced Similar STING, TBK1, IRF3, NFκB Activation in the AQ/SAVI(N153S) and WT/SAVI(N153S) BMDM

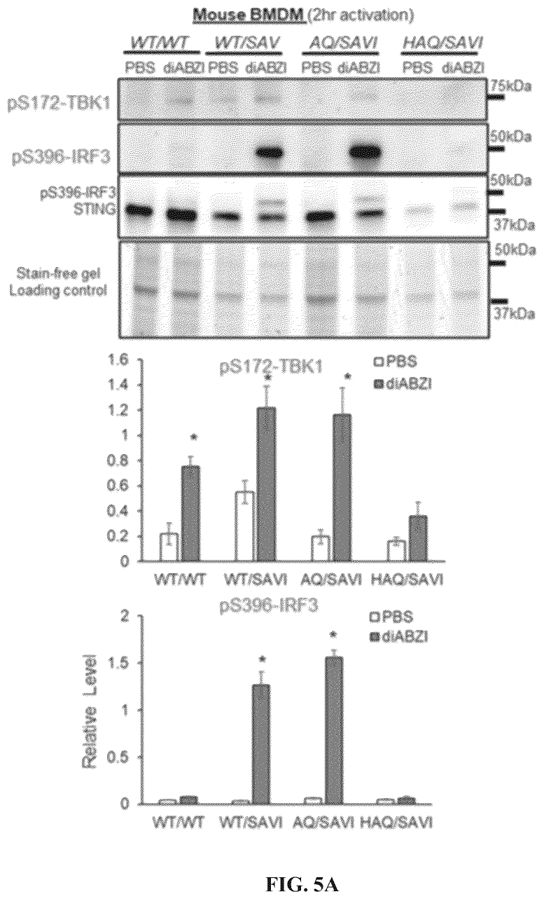

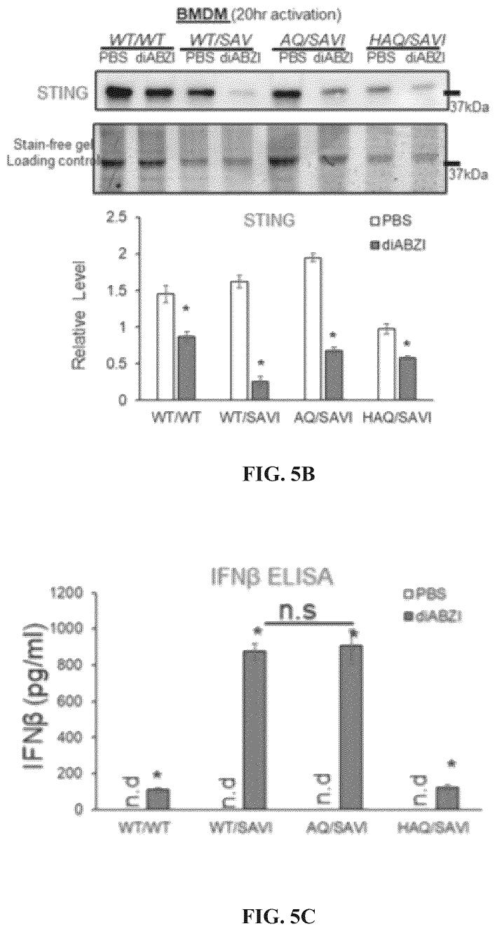

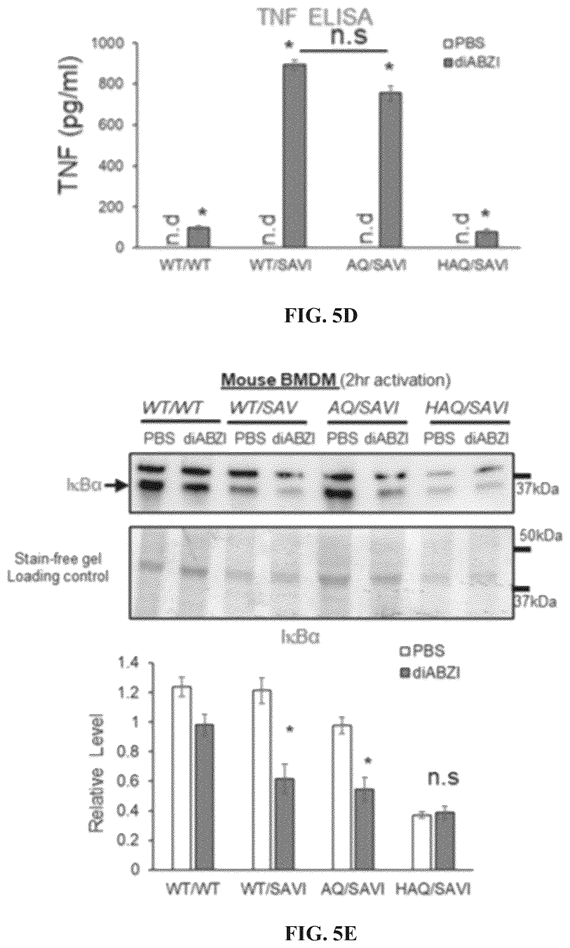

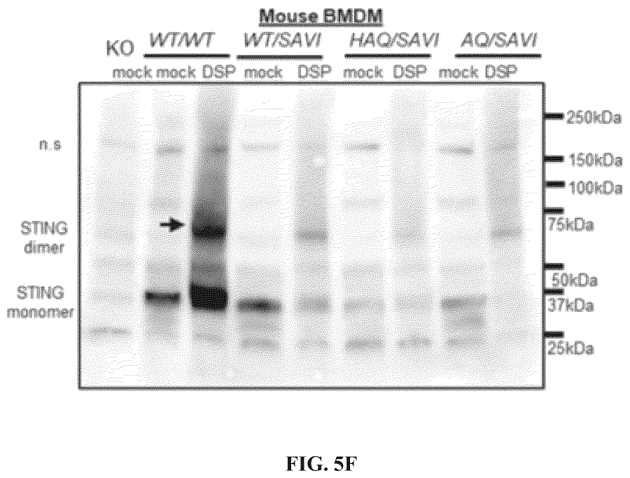

SAVI was characterized as type I interferonopathy (Fremond M. L. et al., J Allergy Clin Immunol Pract 9:803-818, 2021). However, several studies showed that type I IFN signaling and IRF3 activation were dispensable for SAVI disease (Luksch H., et al., J Allergy Clin Immunol 144:254-266, 2019; Stinson W. A., et al., Proc Natl Acad Sci USA 116:7941-7950, 2019; Gao K. M., et al. Proc Natl Acad Sci USA 119, 2022). AQ allele prevented SAVI disease (FIG. 4F-4K). However, diABZI-treated AQ/SAVI(N153S) and WT/SAVI(N153S) BMDM had similar TBK1-IRF3 activation and IFNβ production (FIG. 5a, 5C). diABZI treatment caused IκBα degradation, and similar TNF production in WT/SAVI and AQ/SAVI BMDM (FIG. 5D-5E). Furthermore, diABZI activation led to STING protein degradation in WT/SAVI and AQ/SAVI BMDM (FIG. 5B). Last, using cleavable crosslinker dithiobis (succinimidyl propionate (DSP), it was shown that STING in WT/SAVI, AQ/SAVI BMDM formed similar dimers in situ (FIG. 5F). Thus, the AQ/SAVI BMDM had similar STING degradation, TBK1, IRF3, NFκB activation, and dimerization as the WT/SAVI BMDM.

### Example 12—the HAQ Allele Increased, and the AQ Allele Restored T-Regs in SAVI(N153S) Mice

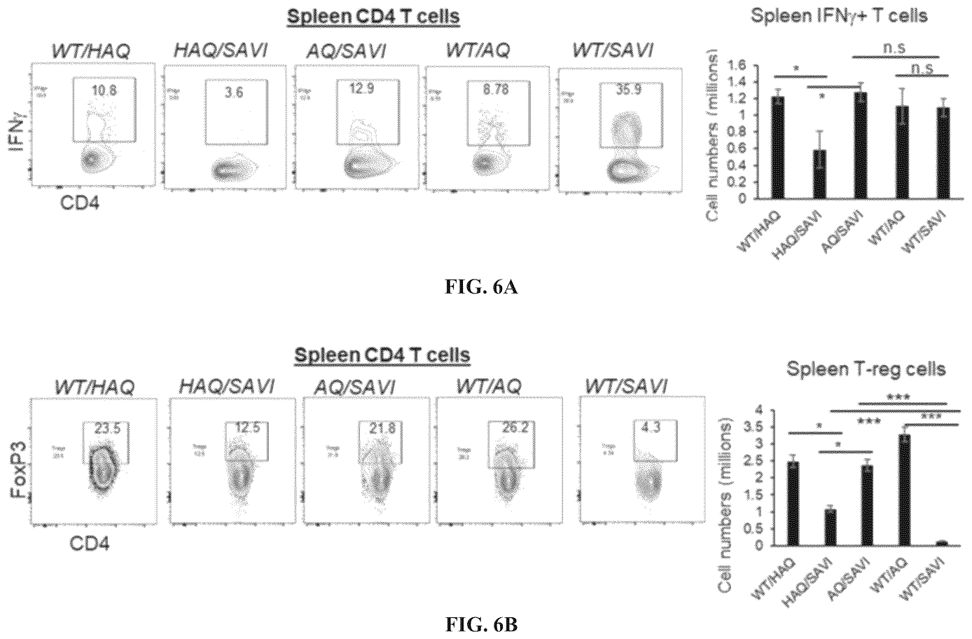

IFNγ was proposed to drive SAVI disease (Stinson W. A., et al., JCI Insight 7, 2022; Patel S., et al., Genes Immun 20:82-89 2019; Gao K. M., et al. Proc Natl Acad Sci USA 119, 2022). It was confirmed that WT/SAVI CD4 T cells were enriched with IFNγ+ cells (FIG. 6A). However, WT/SAVI mice had CD4 T cellpenia. Thus, the total numbers of spleen IFNγ⁺CD4 T cells were comparable in WT/SAVI and AQ/SAVI mice (FIG. 6A). In contrast, the HAQ/SAVI mice had decreased IFNγ⁺CD4 T cells (FIG. 6A). The induction of Foxp3 expression in T-reg cells during ongoing autoimmune inflammation resolved inflammation and pathology in mice (Hu W., et al., Nat Immunol 22:1163-1174, 2021). CD4 T cellpenia depletes CD4 T-regs. Indeed, WT/SAVI mice had ˜20-fold reduction of spleen FoxP3+T-regs compared to AQ/SAVI or WT/WT littermates (FIG. 6B). The HAQ/SAVI mice also had ˜10-fold more T-regs than the WT/SAVI littermate (FIG. 6B).

### Example 13—Discussion

The above study, using the HAQ, AQ, SAVI(N153S) TMEM173 knock-in mice, revealed the in vivo significance and mechanism of STING-mediated CD4 T cell death. HAQ and AQ alleles prevented CD4 T cellpenia and increased/restored CD4 T-regs in SAVI mice.

In HAQ/SAVI and AQ/SAVI mice, one copy of HAQ, AQ allele suppressed CD4 T cell death and targeting STING to prevent CD4 T cell death might be a valid therapy, e.g., for AIDS. Activating the STING pathway is a promising strategy for cancer immunotherapy (Hines J. B., et al., Curr Oncol Rep 25:189-199, 2023; Samson N., et al., Nat Cancer 3:1452-1463, 2022; Liu Y., et al., Eur J Med Chem 211, 2021; Zheng J., et al., Mol Cancer 19, 2020; Corrales L. et al., Cell Rep 11:1018-1030, 2015; Fu J. et al. Sci Transl Med 7, 2015; Barber G. N., et al., Curr Opin Immunol 23:10-20, 2011).

Mechanistically, apoptosis, pyroptosis, ferroptosis, necroptosis, and PANoptosis have all been reported in STING-mediated cell death (Kuhl N. et al., EMBO Rep 24, 2023; Jin L., et al., Mol Cell Biol 28:5014-5026, 2008; Gulen M. F., et al., Nat Commun 8, 2017; Kabelitz D., et al., Sci Rep 12, 2022; Murthy A. M. V., et al., Cell Death Differ 27:2989-3003, 2020; Li C., et al., Sci Rep 12m 2022; Messaoud-Nacer Y., et al., Cell Death Dis 13, 2022; Tang C. H., et al., Cancer Res 76:2137-215225 2016). Different cell types and STING agonists used likely contributed to the inconsistency and complexity. The focus on lymphopenia in SAVI mice as described herein avoided ligand-dependent, non-physiological dosage in STING-mediated cell death. As described herein, HAQ and AQ alleles could prevent CD4 T cellpenia in the SAVI mice, which strongly indicated that residue A230 or Q293 prevented STING-mediated CD4 T cell death in vivo. Splenocyte from Q293 mice were resistant to STING agonists-induced cell death ex vivo. This indicated that the Q293 residue was critical for STING-mediated lymphopenia. Notably, Q293 is outside the C-terminal tail (CTT) (residues 341-379 of human STING) critical for TBK1 recruitment and IRF3 phosphorylation (Liu S. et al. Science 347, 2015) or miniCTT domain (aa343-354) (Cerboni S. et al., J Exp Med 214:1769-1785, 2017), or the UPR motif (aa322-343) (Wu J., et al., J Exp Med 216:867-883, 2019) important for T cell death in vitro. Noteworthy, AQ/SAVI cells had similar TBK1-IRF3, NFκB activation and STING degradation as the WT/SAVI cells. Yet, AQ/SAVI mice did not have CD4 T cellpenia as WT/SAVI mice indicating that the canonical STING-TBK1-IRF3/NFκB pathway, likely STING oligomerization, was not sufficient for the induction of cell death at the physiological condition.

A WT/N153S knock-in SAVI mouse model was used. WT/N153S knock-in SAVI mice spontaneously develop lung inflammation, T cell cytopenia, and early mortality, mimicking pathological findings in human SAVI patients (Warner J. D. et al., J Exp Med 214:3279-3292, 2017). Using the WT/N153S SAVI mouse model and human Jurkat T cell line, it was proposed that STING activation causes chronic ER stress and unfolded protein response, leading to T cell death by apoptosis (Wu J., et al., J Exp Med 216:867-883, 2019). Furthermore, this study showed that crossing WT/N153S mice to the OT-I mice reduced ER stress and restored CD8+, but not CD4+. T cells and that restoration of CD8+ T cells reduced inflammation and lung disease. However, human WT/N154S SAVI patients have normal CD8+ T cells numbers (Liu Y. et al. N Engl J Med 371:507-518, 2014), and primary human CD8+ T cells were largely resistant to STING agonists-induced cell death ex vivo (FIG. 2A). Thus, it was unexpected that restoring CD8+ T cells could rescue SAVI phenotypes since the SAVI patients already have normal CD8+ T cells numbers. Finally, it was unexpected that both HAQ and AQ alleles were resistant to cell death. Previous studies had shown that the HAQ and AQ alleles have opposite functions (Mansouri S., et al., J Immunol 209:2114-2132, 2022). AQ-STING, not HAQ STING, responded to CDNs (Jin L., et al., Genes Immun 12:263-269, 2011; Patel S., et al., J Immunol 198:776-787, 2017; Mansouri S., et al., J Immunol 209:2114-2132, 2022; Patel S., et al., Genes Immun 20:82-89, 2019; Sebastian M. et al., JCI Insight 5, 2020; Yi G., et al., PLOS One 8, 2013; Patel S., et al., J Immunol 198:4185-4188, 20187; Nissen S. K., et al., J Immunol 200:3372-3382, 2018; Ruiz-Moreno J. S. et al., PLOS Pathog 14, 2018; Movert E. et al. Nat Commun 14, 2023). Further, AQ mice are lean while HAQ mice are fat (Mansouri S., et al., J Immunol 209:2114-2132, 2022).

Most importantly, HAQ was positively selected, while AQ was negatively selected, in modern humans outside Africans (Mansouri S., et al., J Immunol 209:2114-2132, 2022). Thus, the death pathway of STING is also distinct from the STING function that was naturally selected. Without wanting to be bound by theory, it is hypothesized that there are at least three unique pathways of STING/MPYS: STING-Type I IFNs, STING-cell death, and STING-fatty acid metabolism and that, as described herein, STING-cell death and CD4 cellpenia can be successfully suppressed by providing dominant Q. AQ, and/or HAQ STING alleles. Furthermore, Q, AQ, and/or HAQ STING alleles could restore Treg cells.

### Example 14—The Q293-STING Allele Cured SAVI in Mice

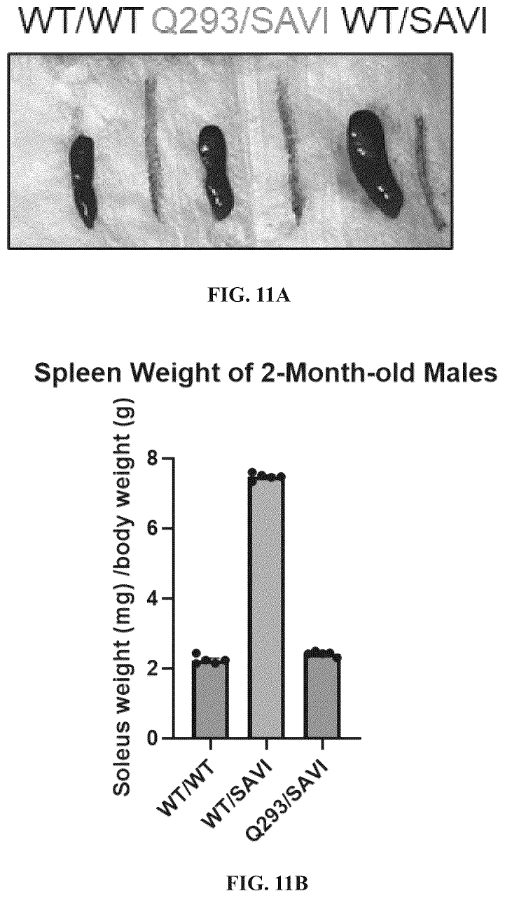

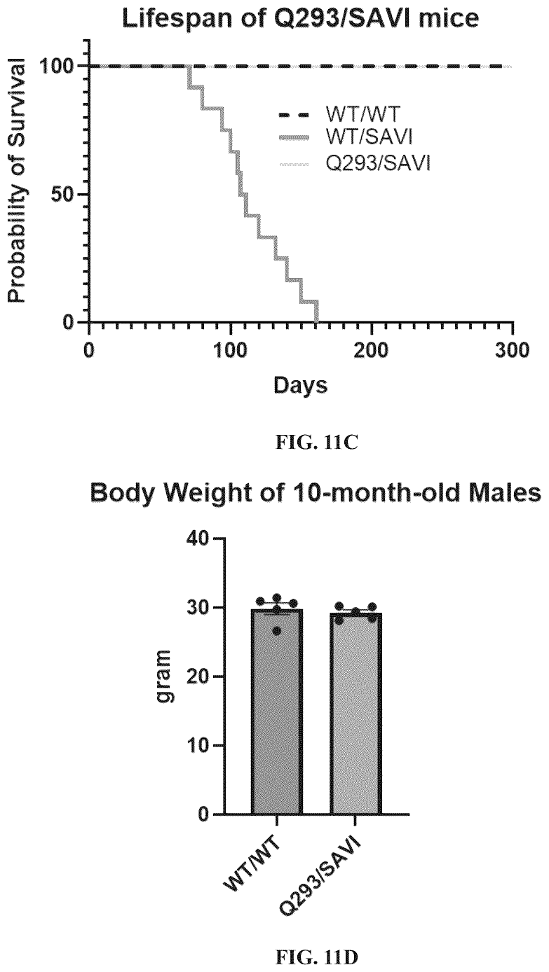

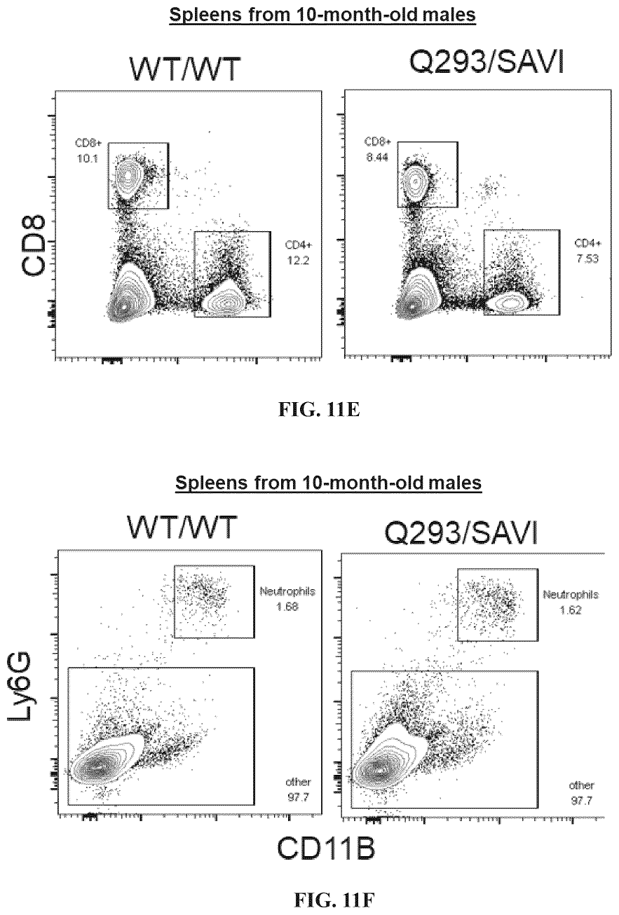

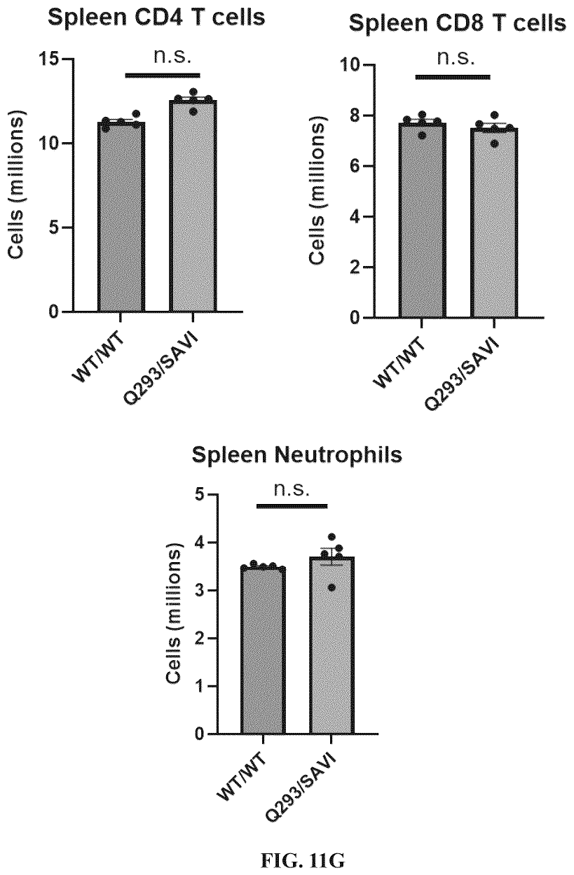

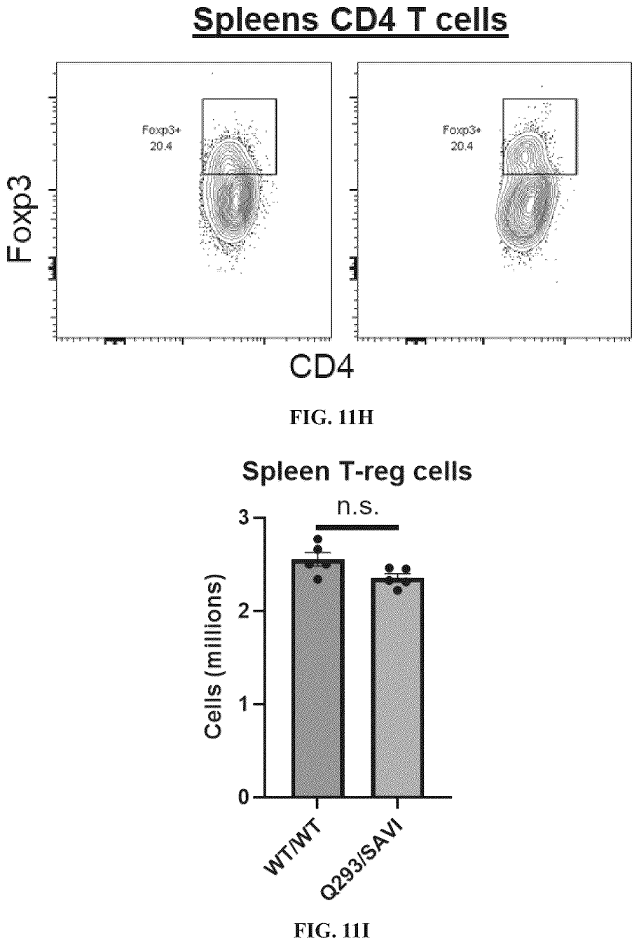

The effects of Q293-STING allele on SAVI in mice were investigated. Three groups were tested: a homozygous wild-type (WT/WT) group, the Q293-STING allele group (Q293/SAVI), and the heterozygous WT/SAVI group (12 mice/group). The weight of spleens from two-month-old mice were determined (FIG. 11B) and exemplary samples were photographed (FIG. 11A). The Q293-STING allele group was comparable to the wild-type group. Similarly, the different strains were monitored for survival for 10 months and the wild-type and Q293/SAVI groups survived for the duration of the study, while the WT/SAVI group did not (FIG. 11C). There was no significant difference in body weight between the wild-type and Q293/SAVI mice at the end of the 10 months (FIG. 11D). Flow cytometry was used to examine splenic CD4 T cells, CD8 T cells, neutrophils, and T regulatory (Treg) cells in wild-type and Q293/SAVI mice at the 10 month time point. No significant difference was observed (FIGS. 11E-11I).

### Example 15—Prediction of Dominant STING Mutations (C148R)

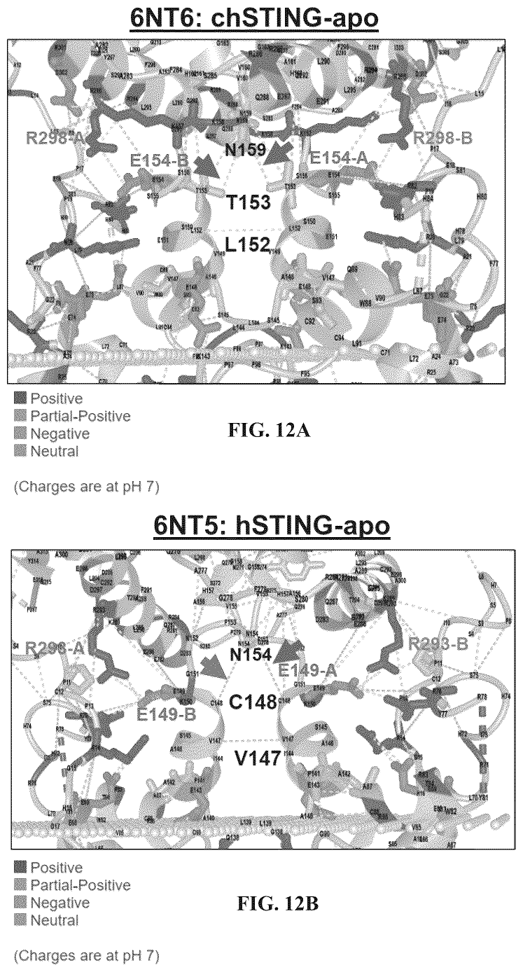

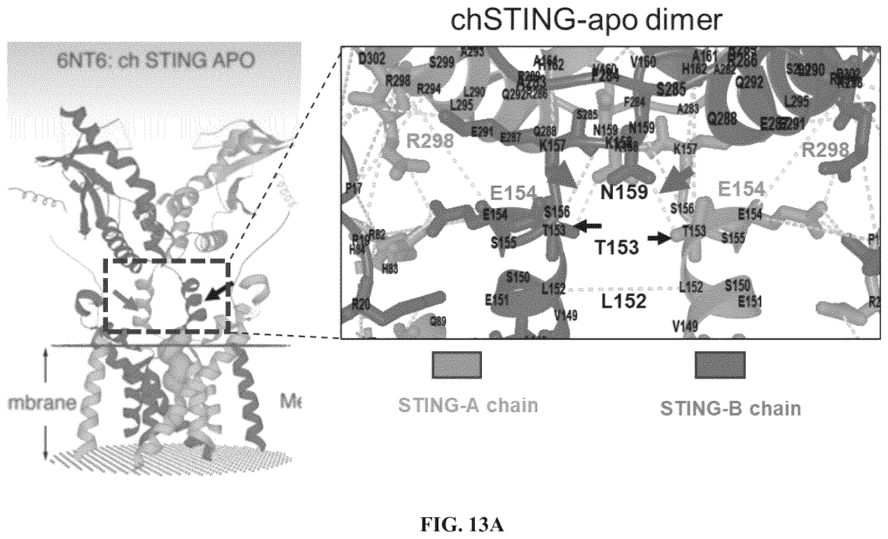

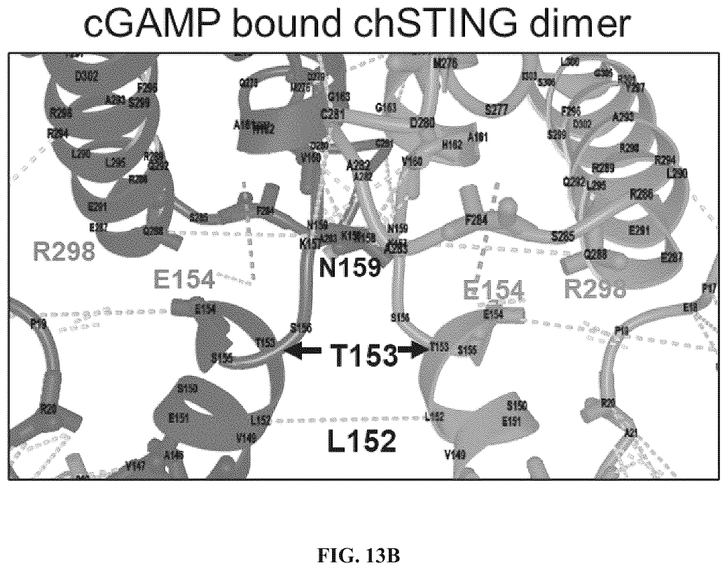

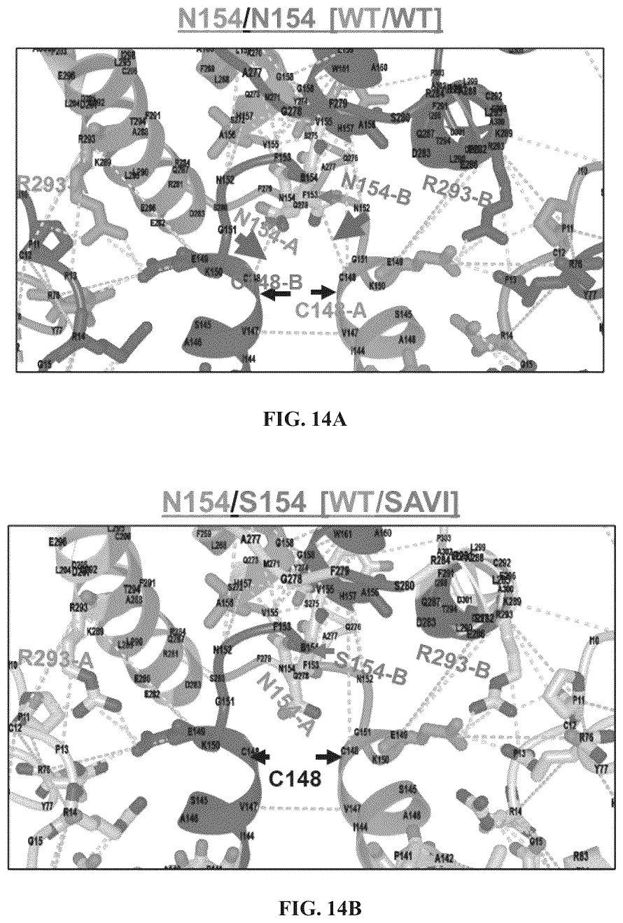

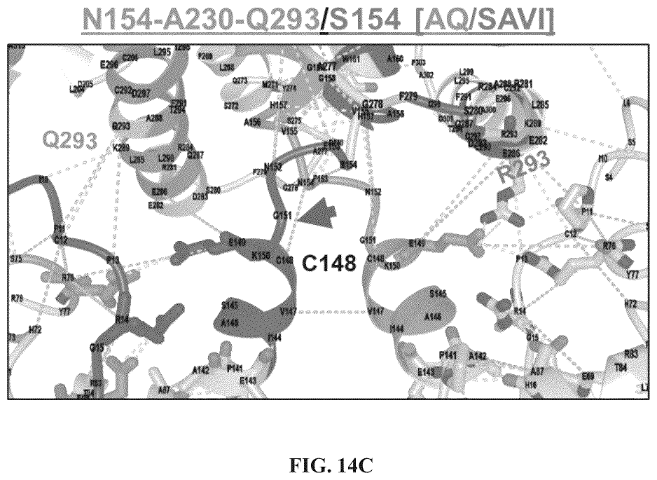

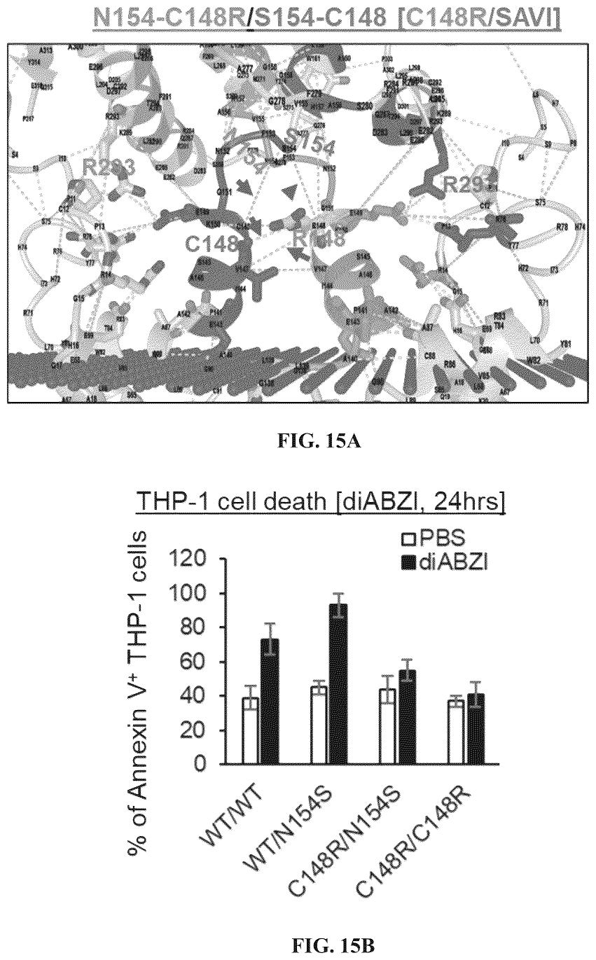

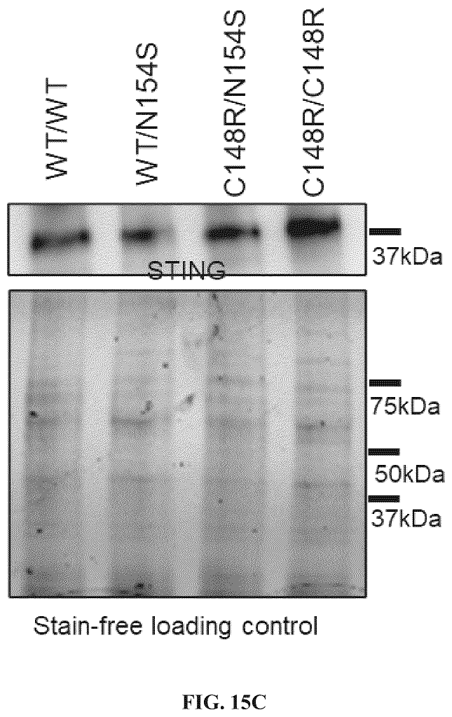

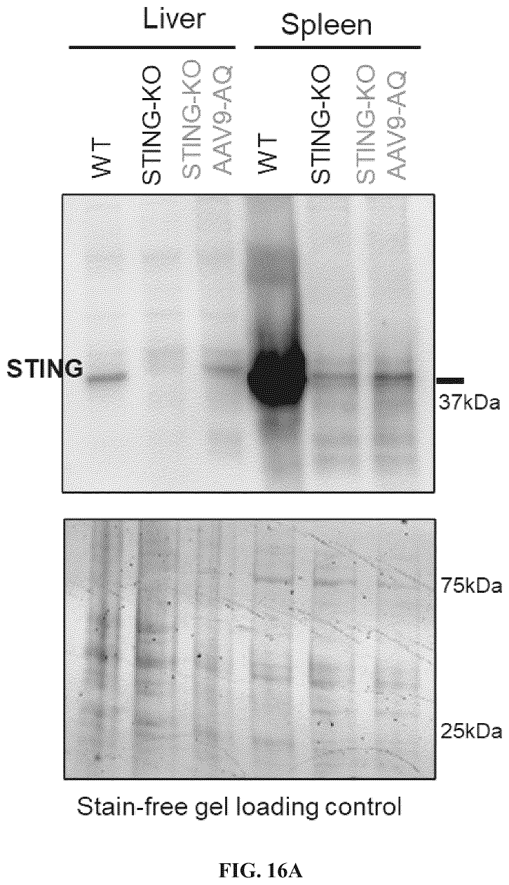

Further work was undertaken to discover additional dominant STING mutations. First, structural information relating to 6NT6 (chicken STING, chSTING-apo; FIG. 12A) and 6NT5 (hSTING-apo; FIG. 12B) was analyzed, and the interactions of the N159-T153/N154-C148 cross-chain were examined, as well as the conserved cross-chain, L152-L152/V147-V147. That is, the cross-chain N154(SAVI)-C148 interaction was conserved between chSTING-apo and hSTING-apo. In addition, cGAMP activation breaks the N159-T153 cross-chain interaction in the chSTING dimer (FIGS. 13A-13C). AQ/SAVI partially restores the N154-C148 cross-chain interaction in the SAVI STING dimer (shown in FIGS. 14A-14C; FIG. 14C depicts partial restoration of the N154-C148 interactions, as marked by an arrow). The resulting mutant, C148R, was then tested to determine whether it suppresses SAVI-STING autoactivation. A structural model is depicted in FIG. 15A. STING-KO human monocytic cell line THP-1 (Invivogen, cat no. thpd-kostg) stably expressing human WT, WT-N154S, C148R-N154S, or C148R/C148R were treated with diABZI (200 ng/ml) in culture for 24 hours. Dead cells were determined by Annexin V staining (FIG. 15B) and expression was confirmed with a Western blot (FIG. 15C). The data confirms that C148R is able to suppress SAVI-STING autoactivation.

### Example 16—Use of rAAV Serotypes to Delivery Dominant STING Mutation to Targeted Organs

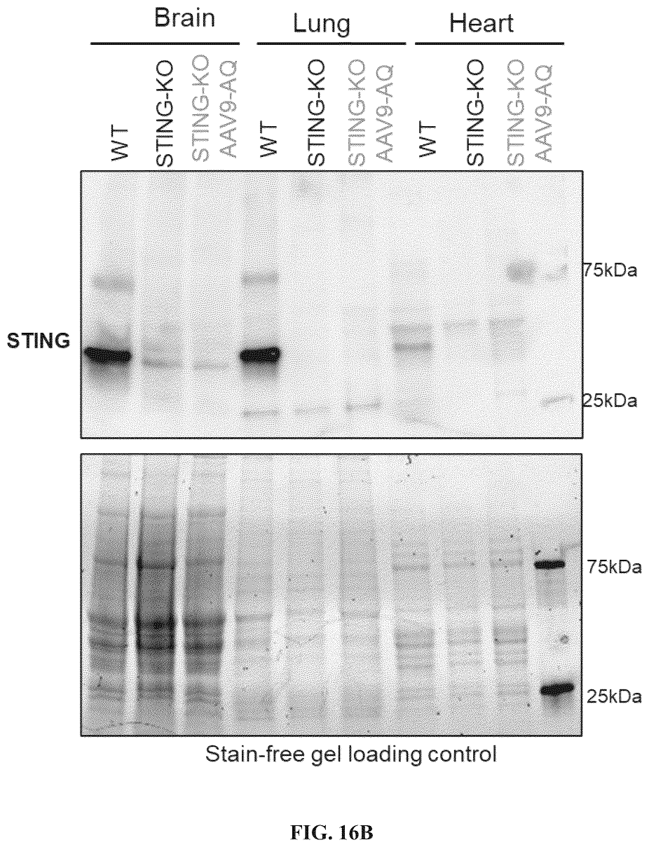

RAAV9-STING (AQ) viral particles (5×10¹¹ genomic copies in 50 μl PBS), which expresses the STING gene with the A230 and Q293 mutations, were injected intravenously into a STING-deficient mouse (STING-KO). Ten weeks post-injection, STING protein expression was determined in harvested organs by Western blot. As is depicted in FIGS. 16A and 16B, rAAV9-AQ restored STING expression in the livers of STING-KO mice, but not in brain, lung, heart or spleen. Data are representative of three independent experiments.

As is known in the art, different rAAV serotypes have different transduction efficacies in different tissues (see, e.g., Table 1 of the supplementary material of et al. Adeno-associated virus serotype rh. 10 displays strong muscle tropism following intraperitoneal delivery. Sci Rep. 2017 Jan. 9:7:40336. doi: 10.1038/srep40336. PMID: 28067312; incorporated herein to the extent it relates to transduction efficacies of different rAAV serotypes).

### Example 17—Identification of Human TMEM173 Genotypes Associated with Alzheimer's Disease

Recent animal studies have revealed STING (Stimulator of interferon genes) as a potential key player in Alzheimer's disease (AD). The actual impact of human STING on AD is; however, unknown. Mouse STING studies were done in WT/WT. However, TMEM173, the human gene encodes STING, has 5 common, distinct, sometimes opposite functional alleles that result in 25 TMEM173 genotypes. Only ˜50% of whites, 36% of African Americans (AA), 22% 25 of East Asians are WT/WT. Past STING cancer immunotherapy clinic trials, which did not consider human TMEM173 heterogeneity, all failed.

The objectives of this study were to: 1) discover new protective and risk AD genetic factors across populations or AA-specific; and 2) establish the physiological significance of common human TMEM173 genotypes and human diseases.

A large-scale (˜15,000 individuals) case-control analysis was conducted between TMEM173 genotypes and AD using data from The National Institute on Aging Genetics of Alzheimer's Disease Data Storage Site. The data included late-onset AD (LOAD) non-Hispanic White (NHW), early-onset AD (EOAD) NHW, and AA.

A common H232/HAQ TMEM173 genotype was found to be associated with AD protection across the populations. An AA-specific TMEM173 genotype H232/Q293 increases the risk for AA males (OR=17.7148), especially in the APOE ε3/ε3 population. The data are shown below. Table 2 shows the TMEM173 genotypes in the case control samples from African Americans.

TABLE 2
TMEM173 Genotypes in Samples from African-Americans

| Healthy-African Americans (median age 72) | | | | | |
|---|---|---|---|---|---|
| | | Population | AD-African Americans (median age 75) | | |
| TMEM173 | Number of | Frequency | TMEM173 | Number of | Population |
| Genotype | Individuals | (%) | Genotype | Individuals | Frequency (%) |
| WT/WT | 534 | 36.0324 | WT/WT | 461 | 36.0156 |
| WT/AQ | 362 | 24.4265 | WT/AQ | 303 | 23.6719 |
| WT/H232 | 241 | 16.2618 | WT/H232 | 199 | 15.5469 |
| WT/HAQ | 82 | 5.5331 | WT/HAQ | 69 | 5.3906 |
| AQ/H232 | 71 | 4.7908 | AQ/H232 | 57 | 4.4531 |
| AQ/AQ | 59 | 3.9811 | AQ/AQ | 58 | 4.5313 |
| WT/Q293 | 47 | 3.1714 | WT/Q293 | 48 | 3.7500 |
| HAQ/AQ | 20 | 1.3495 | HAQ/AQ | 24 | 1.8750 |
| H232/H232 | 14 | 0.9447 | H232/H232 | 20 | 1.5625 |
| AQ/Q293 | 17 | 1.1471 | AQ/Q293 | 13 | 1.0156 |
| HAQ/H232 | 20 | 1.3495 | HAQ/H232 | 7 | 0.5469 |
| H232/Q293 | 3 | 0.2024 | H232/Q293 | 14 | 1.0938 |
| HAQ/Q293 | 3 | 0.2024 | HAQ/Q293 | 2 | 0.1563 |
| HAQ/HAQ | 4 | 0.2699 | HAQ/HAQ | 3 | 0.2344 |
| AQ/A230 | 4 | 0.2699 | AQ/A230 | | 0.0000 |
| WT/A230 | 1 | 0.0675 | WT/A230 | 1 | 0.0781 |
| Q293/Q293 | | 0.0000 | Q293/Q293 | | 0.0000 |
| H232/A230 | | 0.0000 | H232/A230 | 1 | 0.0781 |
| Total | 1482 | 100.0000 | Total | 1280 | 100.0000 |

Table 3 shows a case-control analysis of the H232/HAQ and H232/Q293 genotypes and AD association in African Americans, showing that both genotypes are associated with AD in African-Americans.

TABLE 3
H232/HAQ and H232/Q293 Analysis (in African-Americans)
H232/HAQ and H232/Q293 are associated with AD in African-Americans

| TMEM173 Genotypes (African- | | | | | | |
|---|---|---|---|---|---|---|
| Americans) | WT/WT | H232/H232 | H232/HAQ | HAQ/HAQ | H232/Q293 | Total |
| Number of Individuals - Healthy | 534 | 14 | 20 | 4 | 3 | 1482 |
| Number of Individuals - AD | 461 | 20 | 7 | 3 | 14 | 1280 |
| OR | 0.9993 | 1.6644 | 0.402 | 0.8881 | 5.4518 | |
| p-Value | 0.9927 | 0.1462 | 0.0387 | 0.8532 | 0.0078 | |
| 95% CI | 0.8551~1.1677 | 0.8372~3.3088 | 0.1694~0.9537 | 0.1939~3.8858 | 1.5632~19.014 | |

Table 4 compares the H232/HAQ-AD association in Non-Hispanic White (NHW)-Late Onset Alzheimer's Disease (LOAD), NHW-Early Onset Alzheimer's Disease (EOAD), and African Americans.

TABLE 4
H232/HAQ-AD Association in Different Groups

| | | | | | | Total |
|---|---|---|---|---|---|---|
| | Population frequency | | | p- | | Individuals |
| H232/HAQ | Control | Case | OR | Value | 95% CI | (case + control) |
| NHW-LOAD | 4.62% | 3.53% | 0.7556 | 0.0128 | 0.6059~0.9423 | 9,661 |
| NHW-EOAD | 4.51% | 2.73% | 0.5941 | 0.0221 | 0.3803~0.9280 | 2,498 |
| AA | 1.35% | 0.55% | 0.402 | 0.0387 | 0.1694~0.9537 | 2,762 |

Table 5 compares the H232/Q293-AD association in NHW-LOAD, NHW-EOAD, and African Americans.

TABLE 5
H232/Q293-AD Association in Different Groups

| | | | | | | Total |
|---|---|---|---|---|---|---|
| | Population frequency | | | p- | | Individuals |
| H232/Q293 | Control | Case | OR | Value | 95% CI | (case + control) |
| NHW-LOAD | 0.00% | 0.00% | N.A | N.A | N.A | 9,661 |
| NHW-EOAD | 0.00% | 0.00% | N.A | N.A | N.A | 2,498 |
| AA | 0.20% | 1.09% | 5.4518 | 0.0078 | 1.5632~19.014 | 2,762 |

Table 6 compares the APOE 84/84-AD association in NHW-LOAD, NHW-EOAD, and African Americans.

TABLE 6
APOE ε4/ε4-AD Association in Different Groups

| | | | | | | Total |
|---|---|---|---|---|---|---|
| | Population frequency | | | | | Individuals |
| APOE e4/e4 | Control | Case | OR | p-Value | 95% CI | (case + control) |
| NHW-LOAD | 4.44% | 9.80% | 2.4095 | <0.0001 | 2.0338~2.8546 | 9,471 |
| NHW-EOAD | 0.64% | 17.25% | 32.1385 | <0.0001 | 13.2214~78.1222 | 2,498 |
| AA | 3.29% | 15.28% | 5.3853 | <0.0001 | 3.869~7.4574 | 2,757 |

Table 7 is a case-control analysis of the sex impact on H232/Q293-AD association in African Americans.

TABLE 7
H232/Q293-AD Association in African Americans (sex impact)

| | African - Americans | | | |
|---|---|---|---|---|
| | H232/Q293 | Total | H232/Q293 | Total |
| | females | Females | males | Males |
| Number of | 3 | 1085 | 0 | 396 |
| Individuals - | | | | |
| Healthy | | | | |
| Number of | 6 | 893 | 8 | 387 |
| Individuals - | | | | |
| AD | | | | |
| OR | 2.4424 | | 17.7615 | |
| p-Value | 0.2076 | | 0.0483 | |
| 95% CI | 0.6091 to 9.7940 | | 1.0216 to 308.8095 | |

Table 8 shows a case-control analysis of the APOE impact on H232/Q293-AD association in African Americans.

TABLE 8
H232/Q293-AD Association in African Americans (APOE impact)

| H232/Q293 (African Americans) | APOE e3/e3 | Total | APOE e3/e4 | Total | APOE e4/e4 | Total |
|---|---|---|---|---|---|---|
| Number of Individuals - Healthy | 1 | 659 | 1 | 441 | 0 | 48 |
| Number of Individuals - AD | 6 | 376 | 3 | 564 | 4 | 195 |
| OR | 10.6703 | | 2.3529 | | 2.2794 | |
| p-Value | 0.0287 | | 0.4594 | | 0.5827 | |
| 95% CI | 1.2797 to 88.9728 | | 0.2439 to 22.6989 | | 0.1207 to 43.0617 | |

Accordingly, the H232/HAQ TMEM173 genotype is associated with decreased, while the H232/Q293 TMEM173 genotype is associated with increased, AD risk in humans. In addition, the H232/Q293 TMEM173 genotype selectively increases AD risk in males and APOE3/3 African Americans. The findings demonstrate the first AA-specific high AD risk factor and establish an association between human TMEM173 and AD, paving the way for STING-targeting effective AD healthcare.

### EQUIVALENTS AND SCOPE

While several inventive aspects have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive aspects described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive aspects described herein. It is, therefore, to be understood that the foregoing aspects are presented by way of example only and that, within the scope of the appended claims and equivalents thereto, inventive aspects may be practiced otherwise than as specifically described and claimed. Inventive aspects of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one aspect, to A only (optionally including elements other than B); in another aspect, to B only (optionally including elements other than A); in yet another aspect, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that aspects described in this document using an open-ended transitional phrase (e.g., “comprising”) are also contemplated, in alternative aspects, as “consisting of” and “consisting essentially of” the feature described by the open-ended transitional phrase. For example, if the disclosure describes “a composition comprising A and B,” the disclosure also contemplates the alternative aspects “a composition consisting of A and B” and “a composition consisting essentially of A and B.”

1. A nucleic acid molecule comprising a dominant negative STING allele and an inverted terminal repeat (ITR).

2. The nucleic acid molecule of claim 1, wherein the ITR is an AAV ITR.

3. The nucleic acid molecule of claim 2, wherein the dominant negative STING allele is flanked by two AAV ITRs.

4. The nucleic acid molecule of claim 1, wherein the nucleic acid molecule is a DNA.

5. The nucleic acid molecule of claim 1, wherein the nucleic acid molecule is single stranded.

6. The nucleic acid molecule of claim 1, wherein the nucleic acid molecule is double stranded.

7. The nucleic acid molecule of claim 1, wherein the dominant negative STING allele is selected from a R71H-G230A-R293Q (HAQ) allele, a G230A-R293Q (AQ) allele, a R293Q (Q) allele and a C148R allele.

8. The nucleic acid molecule of claim 1, wherein the nucleic acid molecule further comprises a regulatory sequence.

9. The nucleic acid molecule of claim 8, wherein the regulatory sequence is a promoter, a termination signal, or a combination thereof.

10. The nucleic acid molecule of claim 9, wherein the dominant negative STING allele is operably linked to a promoter.

11. An AAV particle comprising the nucleic acid molecule of claim 1.

12. A composition comprising the nucleic acid molecule of claim 1.

13. A method of suppressing a gain-of-function mutant STING allele in a cell, the method comprising contacting a cell having a gain-of-function mutant STING allele with an effective amount of the AAV particle of claim 11.

14. (canceled)

15. A method of suppressing a gain-of-function mutant STING allele in a subject, the method comprising administering to a subject having a gain-of-function mutant STING allele a therapeutically effective amount of the nucleic acid molecule of claim 1.

16. (canceled)

17. A method of treating STING-associated vasculopathy with onset in infancy (SAVI), the method comprising administering to a subject in need thereof a therapeutically effective amount of the nucleic acid molecule of claim 1.

18. A method of suppressing a STING-mediated inflammatory response in a subject, the method comprising administering to the subject a therapeutically effective amount of the nucleic acid molecule of claim 1.

19. The method of claim 18, wherein the subject is a subject determined to have a STING-mediated inflammatory response.

20. A method of treating a STING-mediated inflammatory disease or disorder in a subject, the method comprising administering to the subject a therapeutically effective amount of the nucleic acid molecule of claim 1.

21. The method of claim 20, wherein the subject is a subject determined to have a STING-mediated disease or disorder.

22. The method of claim 21, wherein the STING-mediated disease or disorder is as Alzheimer's disease, Parkinson's Disease, or amyotrophic lateral sclerosis.


SEQUENCE LISTING (39 sequences)

SEQ ID NO: 1 - protein, 379 aa, Homo sapiens [sequence omitted]

SEQ ID NO: 2 - DNA, 1140 bp, Homo sapiens
  source 1..1140: mol_type = genomic DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 3 - protein, 379 aa, synthetic construct
MPHSSLHPSIPCPRGHGAQKAALVLLSACLVTLWGLGEPPEHTLRYLVLHLASLQLGLLLNGVCSLAEELHHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTADRAGIKDRVYSNSIYELLENGQRAGTCVLEYATPLQTLFAMSQYSQAGFSREDRLEQAKLFCQTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 4 - DNA, 1140 bp, synthetic construct
  source 1..1140: mol_type = other DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcaccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 5 - protein, 379 aa, synthetic construct [sequence omitted]

SEQ ID NO: 6 - DNA, 1140 bp, synthetic construct
  source 1..1140: mol_type = other DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 7 - protein, 379 aa, synthetic construct [sequence omitted]

SEQ ID NO: 8 - DNA, 1140 bp, synthetic construct
  source 1..1140: mol_type = other DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 9 - DNA, 145 bp, unidentified
  source 1..145: mol_type = genomic DNA; note = adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 10 - protein, 736 aa, unidentified
  source 1..736: note = adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - protein, 735 aa, unidentified [sequence omitted]

SEQ ID NO: 12 - protein, 736 aa, unidentified
  source 1..736: note = adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 13 - protein, 734 aa, unidentified
  source 1..734: note = adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 14 - protein, 724 aa, unidentified
  source 1..724: note = adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 15 - protein, 736 aa, unidentified
  source 1..736: note = adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - protein, 737 aa, unidentified
  source 1..737: note = adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 17 - protein, 738 aa, unidentified
  source 1..738: note = adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 18 - protein, 736 aa, unidentified
  source 1..736: note = adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 738 aa, unidentified
  source 1..738: note = adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 20 - protein, 733 aa, unidentified
  source 1..733: note = adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 21 - protein, 742 aa, unidentified
  source 1..742: note = adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 22 - protein, 738 aa, unidentified; identical to SEQ ID NO: 19
  source 1..738: note = rhesus macaques adeno-associated virus 10

SEQ ID NO: 23 - protein, 738 aa, unidentified
  source 1..738: note = rhesus macaques adeno-associated virus 74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 24 - DNA, 2211 bp, synthetic construct
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 25 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 26 - DNA, 2211 bp, synthetic construct
  source 1..2211: mol_type = other DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 27 - DNA, 2285 bp, synthetic construct
  source 1..2285: mol_type = other DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttcc

SEQ ID NO: 28 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 29 - DNA, 2212 bp, synthetic construct
  source 1..2212: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaat

SEQ ID NO: 30 - DNA, 2214 bp, synthetic construct
  source 1..2214: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 31 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctggagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 32 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 33 - DNA, 2217 bp, synthetic construct
  source 1..2217: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 34 - DNA, 2217 bp, synthetic construct
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgccgatcctccgaccaccttcaatcaggccaagctggcttctttcatcacgcagtacagtaccggtcaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 35 - protein, 379 aa, synthetic construct
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVREKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 36 - DNA, 1140 bp, synthetic construct
  source 1..1140: mol_type = other DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgcgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 37 - DNA, 15 bp, synthetic construct
  source 1..15: mol_type = other DNA
gagctgcgccacatc

SEQ ID NO: 38 - DNA, 15 bp, synthetic construct
  source 1..15: mol_type = other DNA
cagaccggtgaccgt

SEQ ID NO: 39 - DNA, 15 bp, synthetic construct
  source 1..15: mol_type = other DNA
ttctgccggacactt